(19) United States
(12) Patent Application Publication
(10) Pub. No.: US 2011/0208215 A1
(43) Pub. Date: Aug. 25, 2011

(54) DEVICES, METHODS, AND KITS FOR FORMING TRACTS IN TISSUE

(76) Inventors: D. Bruce Modesitt, San Carlos, CA (US); Joseph F. Paraschac, Campbell, CA (US); Dan J. Hammersmark, San Mateo, CA (US); David C. Auth, Kirkland, WA (US); Brian Andrew Ellingwood, Sunnyvale, CA (US)

(21) Appl. No.: 12/888,309

(22) Filed: Sep. 22, 2010

Related U.S. Application Data
(60) Provisional application No. 61/244,831, filed on Sep. 22, 2009.

(51) Int. Cl. A61B 17/00; A61B 17/34

(52) U.S. Cl. 606/151; 606/185

(57) ABSTRACT

Tissue tract-forming devices, methods, and kits are disclosed. In some variations, a method for forming a tract in a tissue wall having an interior surface and an exterior surface may comprise advancing an anchor member through the tissue wall and into a lumen defined by the tissue wall, the anchor member comprising a proximal portion, a distal portion, and an intermediate portion therebetween, wherein the proximal and intermediate portions are angled with respect to each other and the intermediate and distal portions are angled with respect to each other, positioning the anchor member so that the intermediate portion contacts the interior surface of the tissue wall and the distal portion is angled toward the interior surface of the tissue wall, and advancing a tissue-piercing member into the tissue wall while the intermediate portion is in contact with the interior surface of the tissue wall, to form a tract in the tissue wall.

### CROSS-REFERENCE TO RELATED APPLICATIONS

This application claims priority under 35 U.S.C §119(e) to U.S. Provisional Application No. 61/244,831, filed Sep. 22, 2009, the disclosure of which is hereby incorporated by reference in its entirety.

### TECHNICAL FIELD

Described here are devices and methods for forming tracts in tissue. More specifically, described here are devices and methods for forming tracts in tissue using at least one anchor member (e.g., to stabilize and/or position the tissue) and at least one tissue-piercing member (e.g., to form the tracts in the tissue).

### BACKGROUND

A number of devices and methods have previously been described for forming tracts in or through tissue. For example, devices and methods for forming tracts in tissue are described in U.S. patent application Ser. Nos. 10/844,247 (published as US 2005/0267520 A1), 10/888,682 (published as US 2006/0009802 A1), 11/432,982 (published as US 2006/0271078 A1), 11/544,149 (published as US 2007/0032802 A1), 11/544,177 (published as US 2007/0027454 A1), 11/544,196 (published as US 2007/0027455 A1), 11/544,317 (published as US 2007/0106246 A1), 11/544,365 (published as US 2007/0032803 A1), 11/545,272 (published as US 2007/0032804 A1), 11/788,509 (published as US 2007/0255313 A1), 11/873,957 (published as US 2009/0105744 A1), 12/467,251 (filed on May 15, 2009), 12/507,038 (filed on Jul. 21, 2009), and 12/507,043 (filed on Jul. 21, 2009), and in U.S. Provisional Application No. 61/178,895 (filed on May 15, 2009), all of which are incorporated herein by reference in their entirety. In some cases, the tracts described there may self-seal or seal without the need for a supplemental closure device. Additionally, the tracts may be quite useful in providing access to a tissue location (e.g., an organ lumen) so that one or more tools may be advanced through a tract, and a procedure may be performed. Given the tremendous applicability of such methods, additional devices and methods for forming tracts in tissue would be desirable.

### BRIEF SUMMARY

Described here are devices, methods, and kits for forming one or more tracts in tissue. In some variations, a tissue tract-forming method may include using an anchor member to stabilize, isolate, and/or position tissue such that one or more tissue-piercing members may be used to form one or more tracts in at least a portion of the tissue. The stabilization, isolation, and/or positioning of the tissue may allow for enhanced control over the tissue and more predictable tract formation than might otherwise occur. In certain variations, an anchor member may alternatively or additionally be used to position a tissue tract-forming device at a target tissue site. The use of the anchor member may, for example, enhance the accuracy of the positioning of the device.

The tracts may be formed in any suitable or desirable tissue. For example, the tissue may be an organ of any of the body systems (e.g., the cardiovascular system, the digestive system, the respiratory system, the excretory system, the reproductive system, the nervous system, etc.). In certain variations, the tissue may be an organ of the cardiovascular system, such as the heart or an artery. In other variations, the tissue may be an organ of the digestive system, such as the stomach or intestines. In some variations, the tissue may be tissue of a vessel wall (e.g., an arterial wall). The devices, methods, and kits may be used in any tissue for which their use is appropriate.

The tracts formed here may seal relatively quickly, without the need for a supplemental closure device. For example, after the tissue-piercing member used to form a tract has been withdrawn from the tract, the tract may self-seal within 15 minutes or less (e.g., within 12 minutes or less, within 10 minutes or less, within 9 minutes or less, within 6 minutes or less, within 5 minutes or less, within 3 minutes or less, within 1 minute or less, etc.). Of course, if necessary or desirable, one or more supplemental closure devices, and/or pressure devices (e.g., manual pressure, pressure applied through a cuff, and the like) may be used in conjunction with the described devices and methods.

In certain variations, a method for forming a tract in a tissue wall (e.g., a vessel wall, such as an artery wall) may comprise advancing at least one tissue-piercing member into the tissue wall to form a tract in the tissue wall, where at least a portion of the tract forms an angle of less than or equal to about 30° (e.g., less than or equal to about 19°, less than or equal to about 15°, less than or equal to about 10°, less than or equal to about 5°, from about 1° to about 30°, from about 1° to about 19°, from about 1° to about 15°, from about 1° to about 10°, from about 1° to about 5°, from about 5° to about 15°, from about 5° to about 10°) with respect to a longitudinal axis of the tissue wall.

During tract formation, a tissue-piercing member may enter tissue at a first location, and exit the tissue at a second location, and the length between the first and second locations may be greater than the thickness of the tissue or the tissue wall (e.g., vessel wall). In certain variations, the length of the tract may be substantially greater than the thickness of the tissue or the tissue wall (e.g., vessel wall), for example, three times, five times, six times, eight times, ten times, etc. greater than the thickness of the tissue or the tissue wall. In some variations, the method may comprise advancing one or more closure devices and/or tools into and/or through the tract.

A tissue-piercing member may be, for example, a needle, such as a hollow needle or a solid needle. The needle may have any suitable tip having any suitable shape. For example, the tip may be conical, offset conical, blunt, sharpened or pointed, beveled, non-beveled, etc.

Some variations of devices and methods described here may be used to deliver one or more therapeutic agents (e.g., drugs) to a target site. For example, a device may be configured to have at least one lumen and one or more apertures (e.g., side ports) in fluid communication with the lumen, such that one or more therapeutic agents may be delivered through the lumen and into a target site via the aperture(s). The therapeutic agent or agents that are used may be selected based on the procedure being performed. As an example, if the target site is stomach tissue, then one or more anti-infective agents may be delivered to the stomach tissue using a device or method described here.

In some variations, a method for forming a tract in a tissue wall having an interior surface and an exterior surface may comprise advancing an anchor member through the tissue wall and into a lumen defined by the tissue wall, the anchor member comprising a proximal portion, a distal portion, and an intermediate portion therebetween. The proximal and intermediate portions may be angled with respect to each other and the intermediate and distal portions may be angled with respect to each other. The method may also comprise positioning the anchor member so that the intermediate portion contacts the interior surface of the tissue wall and the distal portion is angled toward the interior surface of the tissue wall, and advancing a tissue-piercing member into the tissue wall while the intermediate portion is in contact with the interior surface of the tissue wall, to form a tract in the tissue wall. In certain variations, one or more other portions of the anchor member (e.g., the proximal portion and/or the distal portion) may also be in contact with the interior surface of the tissue wall while the tissue-piercing member is advanced into the tissue wall. The distal portion of the anchor member may lift or tent a portion of the tissue wall when the intermediate portion of the anchor member is in contact with the interior surface of the tissue wall. In some variations, the anchor member may be used to stabilize the tissue wall prior to advancement of the tissue-piercing member into the tissue wall.

In certain variations, a device for forming a tract in tissue may comprise a guide, a tissue-piercing member slidably housed within the guide and deployable from the guide through an opening in the guide, and an anchor member coupled to or integral with the guide. The anchor member may comprise a first elongated portion, a second elongated portion that is angled with respect to the first elongated portion, and a third elongated portion that is angled with respect to the second elongated portion. The first elongated portion may define a first plane and the second elongated portion may define a second plane, and the first and second planes may have a first angle of about 1° to about 175° (e.g., about 10° to about 150°, about 10° to about 120°, about 15° to about 100°, about 15° to about 75°, about 20° to about 60°, about 25° to about 50°, about 5° to about 30°, about 6° to about 25°, about 5° to about 20°, about 5° to about 15°, about 5° to about 10°, about 10° to about 20°, about 12°) therebetween.

The first elongated portion may have a length of about 2 millimeters to about 6 millimeters (e.g., about 3 millimeters to about 5 millimeters, or about 4 millimeters). The tissue-piercing member may have a first longitudinal axis and the third elongated portion may have a second longitudinal axis that forms a second angle of about 6° to about 30° (e.g., about 10° to about 25°, about 15° to about 20°) with the first longitudinal axis upon deployment of the tissue-piercing member from the guide. The third elongated portion may define a third plane, and the second and third planes may have a second angle of about 1° to about 175° (e.g., about 10° to about 150°, about 10° to about 120°, about 15° to about 100°, about 15° to about 75°, about 20° to about 60°, about 25° to about 50°, about 5° to about 30°, about 6° to about 25°, about 5° to about 20°, about 5° to about 15°, about 5° to about 10°, about 10° to about 20°, about 12°) therebetween. In some variations, the anchor member may extend distally from the guide.

In certain variations, a device for forming a tract in tissue may comprise a guide, a tissue-piercing member slidably housed within the guide and deployable from the guide through an opening in the guide, and an anchor member coupled to or integral with the guide. The anchor member may comprise first, second, and third elongated portions, a first curved portion between the first and second elongated portions, and a second curved portion between the second and third elongated portions. The first curved portion may define a first plane and the second curved portion may define a second plane that is angled with respect to the first plane. The first and second planes may have an angle of about 1° to about 175° (e.g., about 10° to about 150°, about 10° to about 120°, about 15° to about 100°, about 15° to about 75°, about 20° to about 60°, about 25° to about 50°, about 5° to about 30°, about 6° to about 25°, about 5° to about 20°, about 5° to about 15°, about 5° to about 10°, about 10° to about 20°, about 12°) therebetween. The first and/or second curved portion may have a radius of curvature of about 0.1 millimeter to about 2 millimeters (e.g., about 0.5 millimeter to about 1.5 millimeters). The anchor member may be flexible. In some variations, the anchor member may comprise a guide eye sheath (e.g., in the form of a short tubular portion through which a guidewire may be routed, to help position the guidewire) and/or an attachable guidewire. In some variations, the opening in the guide may be located proximal to a distal end of the anchor member.

In certain variations, a method for forming a tract in a tissue wall having an interior surface and an exterior surface may comprise advancing an anchor member through the tissue wall, the anchor member comprising first, second, and third elongated portions, a first curved portion between the first and second elongated portions, and a second curved portion between the second and third elongated portions, the first curved portion defining a first plane and the second curved portion defining a second plane that is angled with respect to the first plane. The method may also comprise contacting the anchor member with the interior surface of the tissue wall, and advancing a tissue-piercing member into the tissue wall while the anchor member is in contact with the interior surface of the tissue wall, to form a tract in the tissue wall.

The tissue may comprise a vessel (e.g., an artery) and the method may comprise advancing the anchor member into a lumen of the vessel. The tissue-piercing member may have a first longitudinal axis and the third elongated portion of the anchor member may have a second longitudinal axis, and the first and second longitudinal axes may form an angle therebetween. In some variations, the angle between the first and second longitudinal axes may be from about 6° to about 30° (e.g., from about 10° to about 25°, from about 15° to about 20°) when the tissue-piercing member is advanced through the tissue wall. In certain variations, the method may further comprise advancing the tissue-piercing member into a lumen defined by the tissue wall, wherein the angle between the first and second longitudinal axes is from about 6° to about 30° (e.g., from about 10° to about 25°, from about 15° to about 20°) upon entry of the tissue-piercing member into the lumen.

In some variations, a device for forming a tract through tissue may comprise a guide, an anchor member coupled to or integral with a distal portion of the guide, a marker port coupled to or integral with a proximal portion of the guide and having a first lumen, a tissue-piercing member deployable from the guide, and a pushing member configured to deploy the tissue-piercing member from the guide, where the tissue-piercing member comprises a first tubular member comprising a wall portion having a plurality of apertures therethrough, such that the tissue-piercing member is in fluid communication with the marker port. In certain variations, the tissue-piercing member may remain in fluid communication with the marker port when translated by the pushing member.

In some variations, a device for forming a tract through tissue may comprise a marker port comprising a lumen, and a tissue-piercing member comprising a tubular member comprising a wall portion having a plurality of apertures therethrough, where at least a portion of the tissue-piercing member passes through the lumen of the marker port.

In certain variations, a method of forming a tract through tissue using a device comprising an anchor member, a marker port, and a tissue-piercing member at least partially disposed within the marker port and comprising a tubular member comprising a wall portion having a plurality of apertures therethrough may comprise advancing the anchor member into a vessel wall defining a first lumen until blood flows through the marker port to indicate that the anchor member has entered the first lumen. The method may also comprise advancing the tissue-piercing member into the vessel wall while the anchor member is disposed within the first lumen. The tissue-piercing member may comprise a second lumen and the method may further comprise advancing a guidewire through the second lumen. The tissue-piercing member may be advanced into the vessel wall by, for example, pushing on a pushing member that is in contact with the tissue-piercing member.

In some variations, a device for forming a tract through tissue may comprise a guide, a tissue-piercing member deployable from the guide, an anchor member coupled to or integral with the guide, and a sheath coupled to the anchor member. The sheath may comprise a flexible elongated member comprising a distal portion comprising a first region having a first cross-sectional diameter and a second region that is integral with the first region, the second region having a second cross-sectional diameter that is different from the first cross-sectional diameter.

In certain variations, a method of making a device for forming a tract through tissue may comprise forming a sheath using a bump extrusion process, and coupling the sheath to an anchor member that is coupled to or integral with a guide configured for deployment of a tissue-piercing member therefrom. The guide may comprise a lumen and a tissue-piercing member slidably disposed within the lumen.

In some variations, a system for forming a tract through tissue may comprise a syringe and a device comprising a guide, an anchor member coupled to or integral with the guide, a pushing member, and a tissue-piercing member deployable from the guide by pushing on the pushing member. The pushing member may comprise an elongated member having a handle portion at its proximal end, and the syringe may be configured to couple with the handle portion. For example, the handle portion of the pushing member may comprise a female connector and the syringe may comprise a male connector configured to couple to the female connector.

In certain variations, a device for forming a tract in tissue may comprise a guide, a tissue-piercing member slidably housed within the guide and deployable through an opening in the guide, an anchor member coupled to or integral with the guide, a retainer configured to be actuated from a position in which the retainer is aligned with the anchor member to a position in which the retainer extends from the anchor member, and a tensioning apparatus comprising a tensioning member configured to actuate the retainer, and a tubular member housing a portion of the tensioning member. The tubular member may be coupled to or integral with the guide. In some variations, the tensioning member may be coupled to the retainer.

In certain variations, a device for forming a tract in tissue may comprise a guide, a tissue-piercing member slidably housed within the guide and deployable through an opening in the guide, an anchor member coupled to or integral with the guide, a retainer configured to be actuated from a position in which the retainer is aligned with the anchor member to a position in which the retainer extends from the anchor member, and a tensioning apparatus comprising a tensioning member configured to actuate the retainer and a semitubular member housing a portion of the tensioning member. The semitubular member may be coupled to or integral with the guide. In some variations, the tensioning member may be coupled to the retainer.

In certain variations, a device for forming a tract in tissue may comprise a guide, a tissue-piercing member slidably housed within the guide and deployable through an opening in the guide, an anchor member coupled to or integral with the guide, a retainer configured to be actuated from a position in which the retainer is aligned with the anchor member to a position in which the retainer extends from the anchor member, and a tensioning member coupled to the retainer and configured to actuate the retainer. A first portion of the tensioning member may be disposed along an outer surface of the guide, a second portion of the tensioning member may pass through an opening in a wall portion of the guide, and a third portion of the tensioning member may be disposed within a lumen of the guide. The portion of the guide housing the tensioning member may have a non-circular cross-section, such as an elliptical cross-section. The portion of the guide housing the tensioning member may be sized and shaped to house both the tensioning member and the tissue-piercing member.

### BRIEF DESCRIPTION OF THE DRAWINGS

FIG. 1 is a perspective view of a variation of a device for forming one or more tracts in tissue.

FIG. 2A is a side view of a variation of a guide sheath that may be used with devices described herein, and FIG. 2B is a perspective view of the guide sheath of FIG. 2A coupled to a variation of an anchor member.

FIG. 3A is a perspective view of a portion of a variation of the devices described here; FIG. 3B is a perspective view of a delivery guide and anchor member of the device of FIG. 3A; FIG. 3C is a perspective view of the anchor member of FIG. 3B; FIG. 3D is a cutaway perspective view of the delivery guide of FIG. 3B; FIG. 3E is a side view of the portion of the device depicted in FIG. 3A; FIG. 3F illustrates the angles between various components of the device as shown in FIG. 3E; FIG. 3G depicts the lengths of various components of the device as shown in FIG. 3E; FIG. 3H is a top view of the device shown in FIG. 3A; FIG. 31 is an illustration of the angles between various components of the device as shown in FIG. 3H; FIG. 3J is a bottom view of the device shown in FIG. 3A; and FIG. 3K is a front view of the device shown in FIG. 3A.

FIG. 4A is a perspective view of a portion of another variation of a device for forming one or more tracts in tissue; FIG. 4B is a side view of the device depicted in FIG. 4A; FIG. 4C illustrates the angles between various components of the device as shown in FIG. 4B; FIG. 4D depicts the lengths of various components of the device as shown in FIG. 4B; FIG. 4E is a top view of the device shown in FIG. 4A; FIG. 4F is an illustration of the angles between various components of the device as shown in FIG. 4E; FIG. 4G depicts an arrangement of angles that is an alternative to the arrangement shown in FIG. 4F; FIG. 4H is a bottom view of the device of FIG. 4A; and FIG. 41 is a front view of the device of FIG. 4A.

FIG. 5A is a bottom perspective view of a variation of a retainer of a device described herein, and FIG. 5B is an illustrative exploded view of the retainer of FIG. 5A.

FIG. 6A is a perspective view of a variation of a delivery guide of a device for forming one or more tracts in tissue, and FIGS. 6B and 6C depict side and top views, respectively, of the delivery guide of FIG. 6A.

FIG. 6D is a bottom perspective view of a portion of a variation of a device for forming one or more tracts in tissue, where the device comprises a delivery guide, and FIG. 6E is a cross-sectional view of the device of FIG. 6D in the area of the delivery guide, taken along line 6E-6E.

FIG. 6F is a cross-sectional view of a variation of a delivery guide.

FIG. 6G is a bottom perspective view of a portion of a variation of a device for forming tissue tracts, where the device comprises a delivery guide, and FIG. 6H is a cross-sectional view of the device of FIG. 6G in the area of the delivery guide, taken along line 6H-6H.

FIG. 61 is a cross-sectional view of a variation of a delivery guide.

FIG. 7A is a perspective view in partial cross-section of a portion of a variation of a handle of a device for forming one or more tracts in tissue; FIG. 7B is a perspective view in partial cross-section of a portion of another variation of a handle; FIG. 7C is a partial cut-away side view of a portion of a variation of a handle of a device; FIG. 7D is a cut-away view of a portion of the handle of FIG. 7A; and FIG. 7E depicts the alignment between a component of the handle of FIG. 7A and a variation of a syringe configured to couple to the handle.

FIG. 8A is a cross-sectional top view of a variation of a handle of a device for forming one or more tracts in tissue; FIG. 8B shows the handle of FIG. 8A when a pushing member of the handle is prevented from moving distally; FIG. 8C shows the handle of FIG. 8A when the pushing member is capable of moving; FIG. 8D depicts the handle of FIG. 8A after the pushing member has been distally advanced; and FIG. 8E shows the handle of FIG. 8A after the pushing member has been retracted.

FIG. 8F is a cutaway top view of a portion of the housing of the handle of FIG. 8A, and FIGS. 8G and 8H show a portion of the housing of FIG. 8F and depict the movement of a component of the handle within the portion of the housing during use.

FIGS. 8I-8L show another portion of the housing of FIG. 8F, at different times as a pushing member of the handle is moved during use.

FIGS. 8M and 8N show a portion of another variation of a handle housing of a device for forming one or more tracts in tissue.

FIG. 8O is a cutaway top view of a portion of a variation of a handle of a device for forming one or more tracts in tissue.

FIGS. 9A and 9B are top perspective views of variations of devices for forming one or more tracts in tissue.

FIGS. 10A-10C depict a Seldinger method for forming an opening in a vessel wall, and FIGS. 10D-10H depict one variation of a method for forming a tract through the vessel wall using a device positioned within the opening.

FIGS. 11A and 11B depict one variation of a method for positioning and/or stabilizing a vessel wall.

FIGS. 12A and 12B depict one variation of a method for forming a tract through a vessel wall once the vessel wall has been positioned and/or stabilized.

FIGS. 13A-13E depict different variations of tracts through a vessel wall.

### DETAILED DESCRIPTION

Described here are devices and methods for forming one or more tracts in tissue. The tract or tracts may be used, for example, to advance one or more tools to a target site, such as a lumen of the tissue. In general, tracts formed by the devices and methods described here may seal relatively quickly, and/or may seal without the need for a supplemental closure or pressure device. Moreover, the devices may be used to form tissue tracts in a relatively controlled manner. In some variations, the devices may comprise one or more anchor members (e.g., having a shape similar to that of a ski tip or a corkscrew) that may be used to position and/or stabilize tissue for tract formation, and/or to accurately position the devices relative to the tissue during tissue tract formation. In certain variations, the tissue may be positioned and/or stabilized for advancement of a tissue-piercing member therethrough. Such positioning and/or stabilization may allow for relatively accurate, easy, and efficient tract formation.

In some variations, the devices and/or methods described here may further include one or more other features that may enhance their ease of use and efficiency. As an example, the devices may provide a visual indication of entry into a target site, such as a blood flash upon entry into a vessel lumen. Such an indication may be provided without adversely affecting tissue tract formation. As another example, the devices may be configured to couple with one or more syringes relatively easily, such as when the devices are in use. For example, a device may be configured to couple with a saline-filled syringe, which may be used to flush the device with saline, and/or flush a vessel lumen. Alternatively or additionally, a device may be configured to couple with a syringe that may then be used to deliver one or more therapeutic agents through the device. The devices may also be configured for relative ease of use. Moreover, the components of the devices may be arranged in such a way as to maintain a low overall profile. Additionally, in some variations, one or more components of the devices, or the devices themselves, may be manufactured relatively easily and efficiently.

It should be understood that the devices and methods described here may be used with any tissue in which it is desired to form one or more tracts. For example, the tissue may be an organ, such as an organ of any of the body systems (e.g., the cardiovascular system, the respiratory system, the excretory system, the digestive system, the reproductive system, the nervous system, etc.). In some variations, the tissue may be an organ of the digestive system, such as the stomach or intestines. In other variations, the methods may be used with tissue of the cardiovascular system, such as the vasculature (e.g., an artery) or the heart. As an example, one or more tracts may be formed through a muscular wall and/or septum of a heart to access the left ventricle, the aorta, the aortic valve, the mitral valve, the aortic arch, etc. For example, a tissue-piercing member may be used to form a tract from a peripheral surface of a heart, through a muscular wall of the heart, and into a septum of the heart. In certain variations, a tissue-piercing member may be used to form a transapical tract into a heart. In some variations, the tissue may be an artery, and the methods may be used in conjunction with performing an arterial puncture (e.g., an arteriotomy). In certain variations, the tissue may be accessed through a natural orifice (e.g., to perform natural orifice translumenal endoscopic surgery, or “NOTES”). The tissue may be, for example, tissue of the reproductive system, excretory system, digestive system, or the like. Of course, it should be understood that methods of forming multiple tracts in tissue, whether through similar or different tissue, are also contemplated.

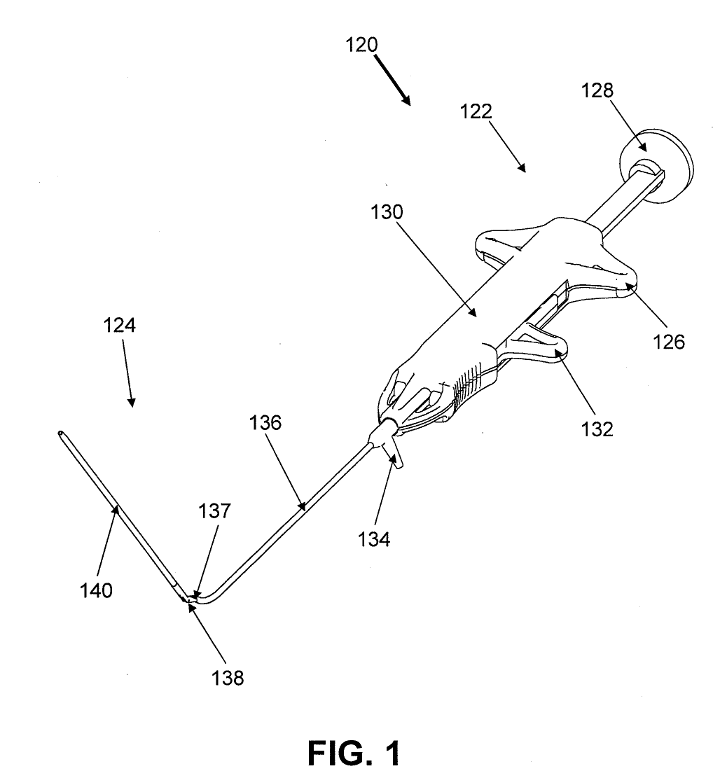

FIG. 1 depicts one variation of a device (120) that may be used to form one or more tracts in tissue (e.g., in accordance with the various methods described here), for example, to form a tract through an arterial wall. As shown there, device (120) has a proximal portion (122), which generally will be located outside body tissue during use, and a distal portion (124), at least a portion of which will generally be located within body tissue during use. Proximal portion (122) comprises a handle (126), a pushing member (128) (e.g., a plunger), and a housing (130), as well as an actuator (132), and a marker port (134). Distal portion (124) comprises a delivery guide (136) having a lumen (not shown) for housing a tissue-piercing member (also not shown), a tissue-piercing member port (137), an anchor member (138), a guide sheath (140), and a retainer (shown and described below). One or more levers, buttons, slide actuators, dials, knobs, etc. may also be included in the proximal portion, as suitable, and may be used, for example, to control the various components of the device, and/or to ensure that certain device components are used in a particular sequence. Each component will now be described in detail.

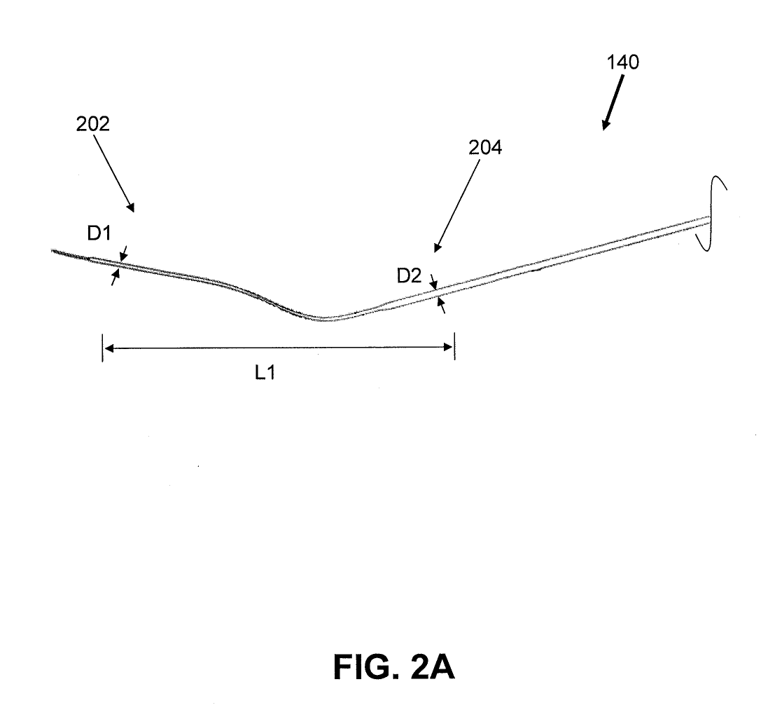

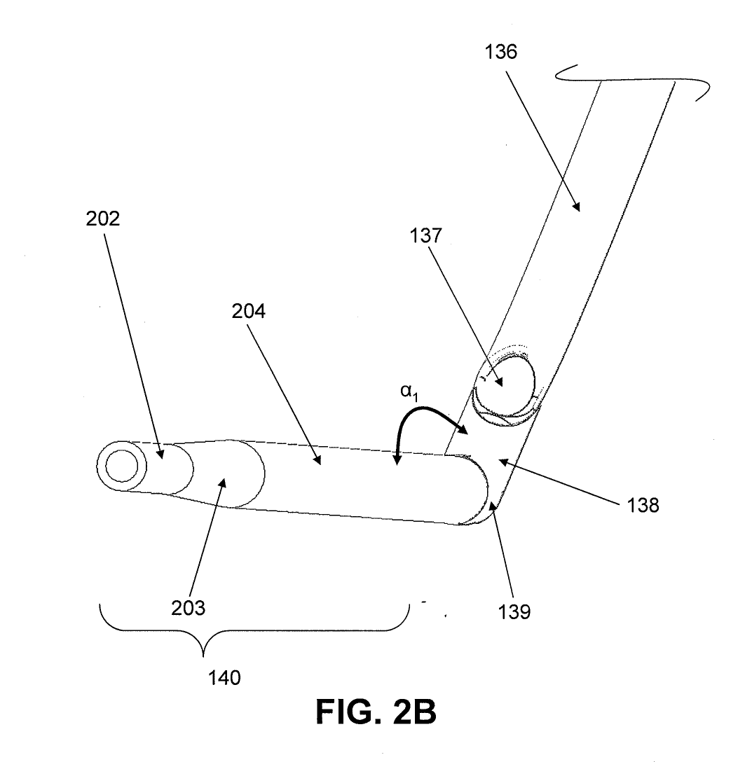

Guide sheath (140) is depicted in FIG. 2A. During use, guide sheath (140) may aid in the advancement of device (120) to a target site, as well as the positioning of device (120) once at the target site. For example, guide sheath (140) may be advanced over a guidewire and into a lumen of an artery in which device (120) will be used to form an arteriotomy.

As shown in FIG. 2A, guide sheath (140) has a distal portion (202) and a proximal portion (204). In some variations, the proximal and distal portions may have different material properties from each other. For example, distal portion (202) may be more flexible than proximal portion (204). A relatively flexible distal portion may, for example, be unlikely to cause tissue damage. A configuration in which distal portion (202) is more flexible than proximal portion (204) may also allow for responsive navigation of guide sheath (140). In some variations, proximal portion (204) may alternatively or additionally be rigid and firm. This may, for example, promote tissue engagement and stabilization by providing a relatively firm structure against which the tissue may be contacted, as well as allowing for relatively easy tracking and good pushability. Certain variations of a guide sheath may have one or more side openings or slits, sized and shaped for the passage of a guide element (e.g., a guidewire) therethrough. Alternatively or additionally, an anchor member may comprise one or more of such side openings or slits.

Guide sheath (140) has a length (L1), a dimension (D1) (e.g., a cross-sectional diameter) in distal portion (202), and a dimension (D2) (e.g., a cross-sectional diameter) in proximal portion (204) that is greater than dimension (D1). It should be understood, however, that other variations of guide sheaths may be relatively uniform in size along their length, such that they do not exhibit this variation in dimensions, or may have more than two portions with different dimensions (e.g., different cross-sectional diameters). Additionally, in some variations, a guide sheath may have a proximal portion with a smaller dimension (e.g., cross-sectional diameter) than its distal portion. The dimensions and configuration of a guide sheath may depend, for example, on the procedure or procedures for which the guide sheath is to be used, and/or on the characteristics of the target tissue.

In some variations (e.g., some variations in which guide sheath (140) is inserted into an arterial lumen), length (L1) may be from about 10 millimeters to about 400 millimeters (e.g., from about 50 millimeters to about 300 millimeters, from about 100 millimeters to about 200 millimeters). Alternatively or additionally, dimension (D1) may be from about 0.2 millimeter to about 2 millimeters (e.g., from about 1 millimeter to about 1.5 millimeters), and/or dimension (D2) may be from about 0.5 millimeter to about 3 millimeters (e.g., from about 1.5 millimeters to about 2 millimeters). The transition between differently sized guide sheath portions may be relatively gradual and tapered, or may be sharper. The characteristics of the transition may depend, for example, on the desired features of the guide sheath.

Guide sheath (140) may comprise any suitable material or materials. As an example, in some variations, guide sheath (140) may comprise one or more polymers or polymer composites, or combinations (e.g., blends) thereof. In certain variations, guide sheath (140) may comprise one or more porous materials, such as expanded polytetrafluoroethylene (ePTFE), and/or one or more substantially non-porous materials, such as polyether block amide (PEBAX™) or polyethylene. In some cases, a guide sheath comprising one or more porous materials may be used to release one or more therapeutic agents as the guide sheath is advanced through the tissue. Non-porous materials may be used, for example, to reduce the surface area of the guide sheath that is exposed to the tissue. Certain variations of a guide sheath may also have one or more coatings, where the one or more coatings may help to enhance tract formation, for example, to promote smooth, low-friction tract formation. In some variations, the guide sheath may be coated with a therapeutic agent, such as agents that may help to seal the tract after the guide sheath has been withdrawn, or agents that may be delivered to a vessel lumen for the treatment of various diseases, e.g., anti-inflammatory agents, anti-thrombosis agents, etc., or for other purposes, such as a contrast agent for imaging. These agents may also be delivered to a vessel lumen via one or more ports or openings in a guide sheath (not shown). The material chosen for the guide sheath, as well as the type and number of ports or openings that are provided, may be determined at least in part by the desired rate of agent delivery.

In some variations, a guide sheath may comprise multiple different sections that are coupled to each other or that are integral with each other (e.g., formed by a coextrusion process). In certain variations, two or more of the sections may have the same structural, material, and/or mechanical properties. Alternatively or additionally, in some variations, two or more of the sections may have different structural, material, and/or mechanical properties. As an example, a guide sheath may comprise a distal section that is relatively flexible and that has a relatively small diameter, as well as a proximal section that is relatively rigid and that has a relatively large diameter. As another example, a guide sheath may comprise different sections having different durometers. The different sections of a guide sheath may be coupled to each other in any suitable fashion, such as by heat-bonding, adhesive-bonding, mechanical or living hinges, form-fitting, screw-fitting, snap-fitting, brazing, soldering, welding, and the like.

In some variations, a guide sheath such as guide sheath (140) (FIG. 2A) may be made using a bump extrusion process. For example, guide sheath (140) may be made from a single material that is bump extruded, such that distal portion (202), with its smaller diameter, is relatively flexible, while proximal portion (204), with its larger diameter, is relatively rigid. A guide sheath that is formed using a bump extrusion process may comprise a single material, or may comprise a combination of two or more materials, such as a blend of different polymers. Non-limiting examples of suitable materials include PEBAX™, polyethylene, or PTFE. When a bump extrusion process is used, the resulting guide sheath may, for example, not have any joints or discontinuities along its length (e.g., between its proximal and distal sections). Additionally, the bump extrusion process may allow for continuous diameter variation across the guide sheath. Moreover, the resulting guide sheath may be unlikely to experience separation of one of its sections from another section.

Of course, while the use of bump extrusion has been described, other appropriate methods may also be used to make a guide sheath, including but not limited to fiber spinning methods, injection molding methods, and any other suitable extrusion or molding methods.

In some variations, a guide sheath may include one or more slots along its length and/or around its circumference. The slots may, for example, enhance the flexibility and/or navigational capability of the guide sheath, or may be used for delivering various agents as described above. In certain variations, a guide sheath may be steerable. Such steerability may be controlled, for example, using an actuator located proximal to the guide sheath (e.g., by urging actuator (132) toward handle (126), FIG. 1). Steerability may allow at least a portion of a guide sheath to conform to a tissue's shape in real time. For example, a steerable guide sheath may be desirable for accessing and forming a tract through an arterial wall. Alternatively or additionally, a guide sheath may be pre-shaped with one or more curves as suitable for the tissue to be accessed. In some variations, a guide sheath may include one or more lumens therethrough—the lumens may be connected to one or more ports in the guide sheath, and may be used, for example, to deliver one or more therapeutic agents and/or a saline flush through the guide sheath. The therapeutic agents and/or saline flush may be introduced to a lumen of a guide sheath via a syringe (or any suitable reservoir) near the proximal portion (122) of the device, for example, via the pushing member (128) or the marker port (134). In some variations, the delivery of the one or more therapeutic agents may be computer controlled or pre-programmed.

FIG. 2B depicts one way in which guide sheath (140) may be connected to anchor member (138). As shown there, proximal portion (204) of guide sheath (140) is coupled to a distal portion (139) of anchor member (138). In FIG. 2B, guide sheath (140) is coupled to anchor member (138) via core wire that is crimped to the distal portion (139). The core wire may be melted to fuse guide sheath (140) to anchor member (138). However, in other variations, a guide sheath may be coupled to an anchor member using mechanical junctions, form-fitting, screw-fitting, snap-fitting, adhesive-bonding, brazing, welding, soldering, and the like, or the guide sheath and anchor member may be integral with each other. Typically, it is desirable for the coupling between a guide sheath and an anchor member to be secure (e.g., to prevent dissociation during use).

Referring again to FIG. 2B, guide sheath (140) may be coupled to anchor member (138) at an angle (α₁), which may be from about 0° to about 360° (e.g., from about 5° to about 270°, from about 15° to about 270°, from about 45° to about 270°, from about 45° to about 180°, from about 45° to about 150°, from about 90° to about 180°, from about 90° to about 150°, from about 45° to about 90°, from about 6° to about 30°, or about 180°). The angle between a coupled guide sheath and anchor member may be selected, for example, based on the characteristics of the target tissue. In some variations, the distal portion of anchor member (138) may have an angle (α₁), and the guide sheath (140) may be aligned with the anchor member (i.e., the guide sheath may be straight with respect to the anchor member). Additionally, and as described previously, a guide sheath may vary in its dimensions along its length. For example, a proximal portion of the guide sheath may have a larger diameter than a distal portion of the guide sheath. Moreover, and referring again to FIG. 2B, guide sheath (140) may include a bump section (203) between proximal portion (204) and distal portion (202), where guide sheath (140) may transition from a larger dimension (D2) to a smaller dimension (D1).

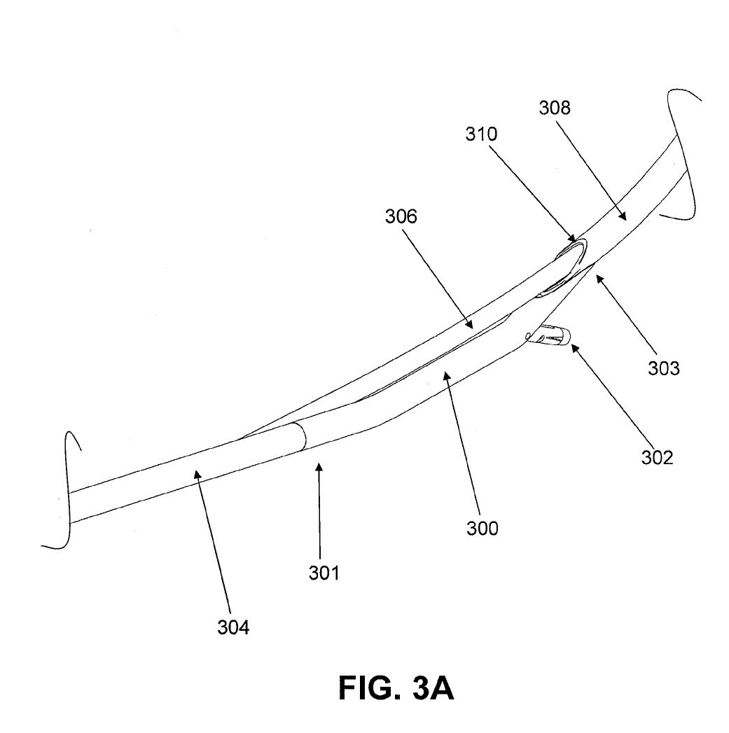

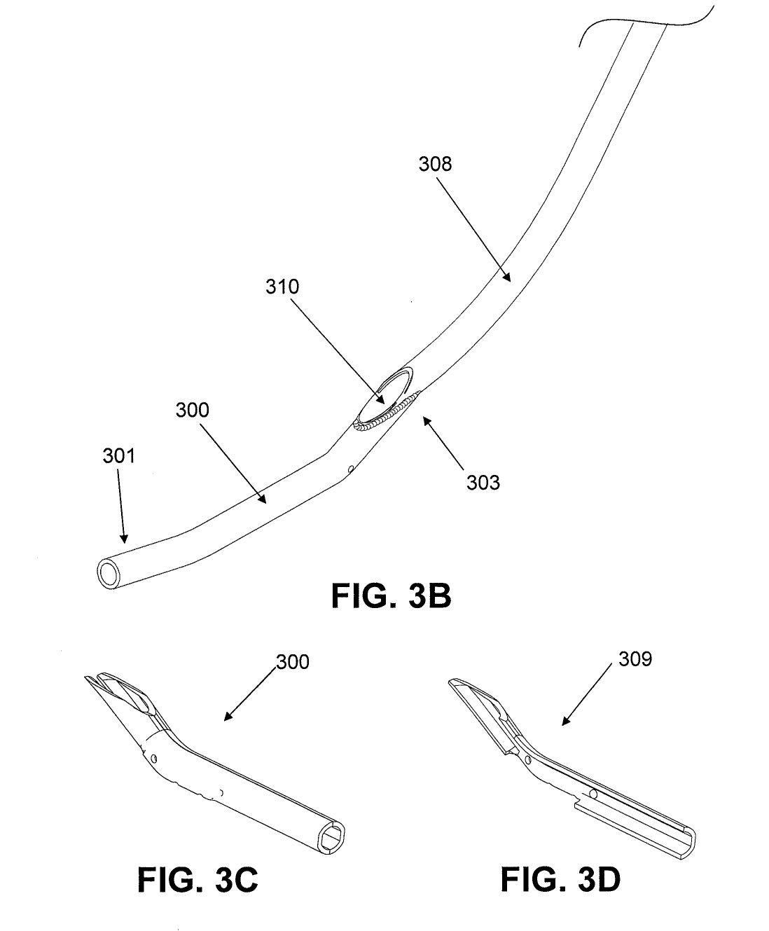

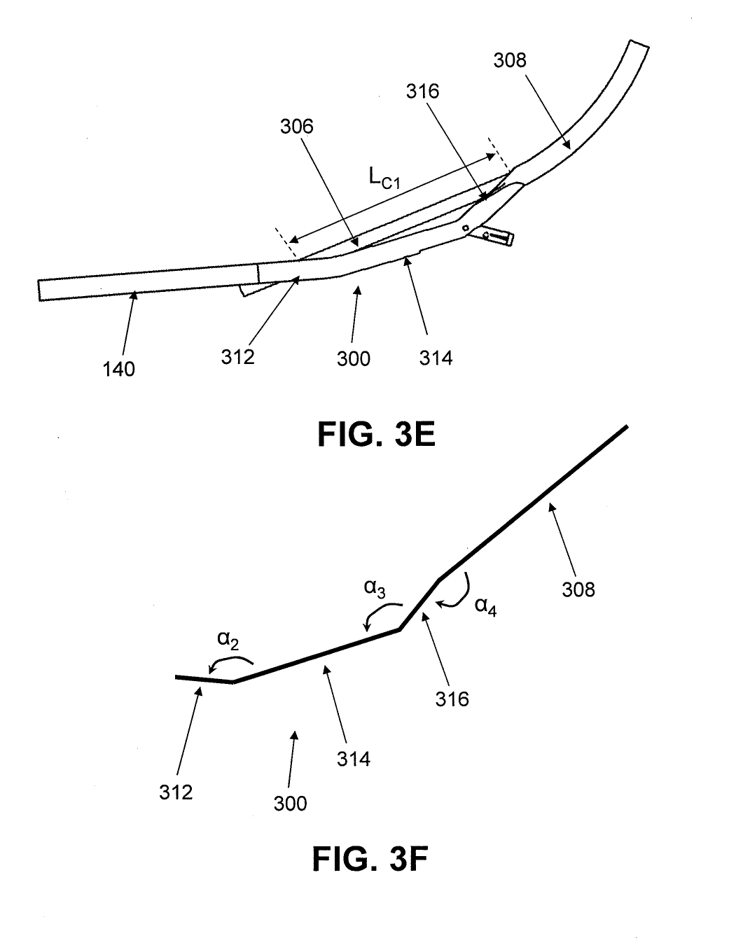

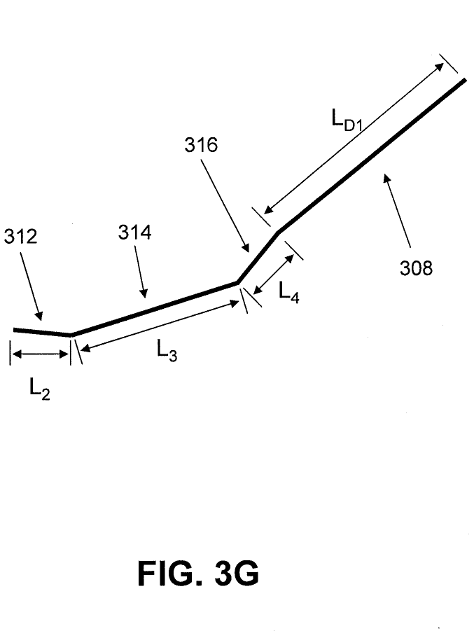

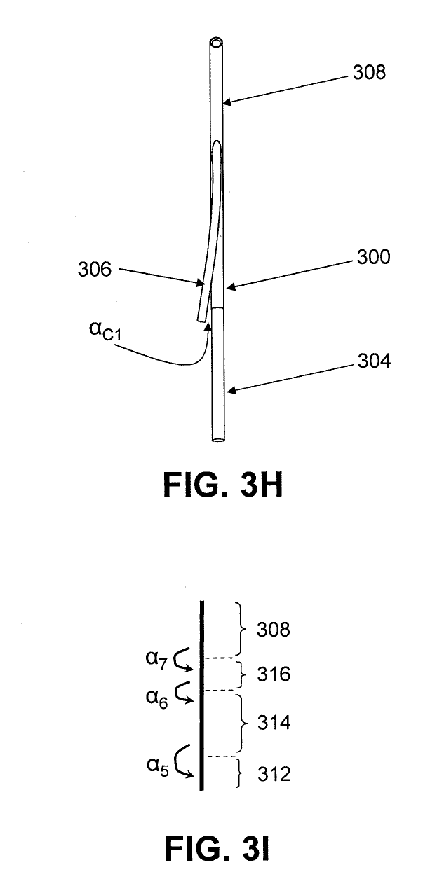

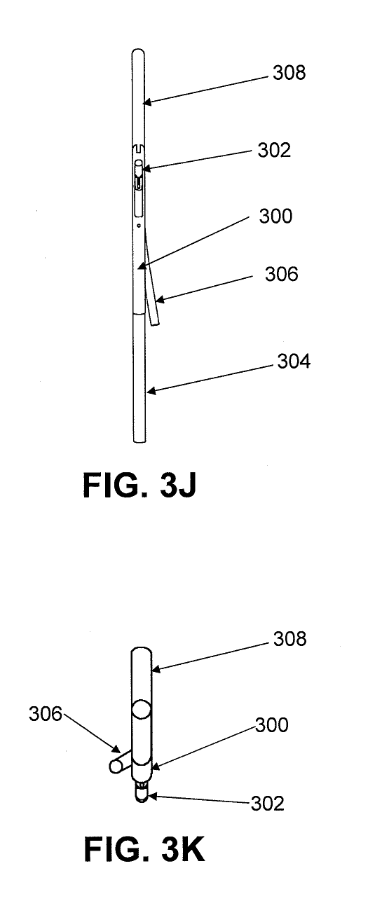

An anchor member for use in a tissue tract-forming device may have any size, shape, and configuration that are appropriate for the particular method and/or target tissue, for example, forming a tract through the wall of an artery. FIGS. 3A-3K depict two different exemplary variations of anchor members, although it should be understood that any other suitable variation may also be used.

First, FIGS. 3A and 3B depict an anchor member (300) having a shape similar to a ski tip, such that its distal portion tilts upward with respect to its more proximal portion, as will be discussed in further detail below. Anchor member (300) comprises a distal portion (301) at which the anchor member is attached to a guide sheath (304). Anchor member (300) also comprises a proximal portion (303) at which the anchor member is attached to a delivery guide (308) having a lumen that terminates at tissue-piercing member port (310). The lumen may generally be sized and shaped for housing a tissue-piercing member. FIG. 3A further depicts a tissue-piercing member (306) that is slidably housed within the lumen of delivery guide (308), and that has been advanced from delivery guide (308) through a tissue-piercing member port (310). The path of tissue-piercing member (306) with respect to anchor member (300) as tissue-piercing member (306) is advanced will be described in detail below. Anchor member (300) also comprises a retainer (302), the function(s) of which will be discussed in further detail below.

FIGS. 3C and 3D depict one possible configuration of anchor member (300). First, FIG. 3C shows anchor member (300) in its assembled form and detached from delivery guide (308). Anchor member (300) may, for example, be in the form of two molded pieces that have been fitted together and welded, or otherwise securely coupled using mechanical junctions, form-fitting, screw-fitting, snap-fitting, adhesive-bonding, brazing, soldering, and the like. The pieces that form anchor member (300) may be stamped, forged, or otherwise formed by any method, such as a powdered metal process, or a metal injection molding process. An example of such a molded piece (309) is shown in FIG. 3D. The second molded piece (not shown) may, in some cases, be a mirror image of the first molded piece (309), and may be welded, soldered, form-fit, screw-fit, snap-fit, adhered, brazed, etc. to the first molded piece to form the anchor member. Other appropriate coupling methods may also be used. The method by which an anchor member is made may depend, for example, on the geometry of the anchor member, and/or its desired structural characteristics. As an example, if it is desired that an anchor member be able to withstand strong compressive forces, then the anchor member may be integrally formed (i.e., as one piece), to limit the likelihood of any breakpoints or frangible regions being present in the anchor member.

Anchor members may have any appropriate configuration, and in some cases may have one or more curves. The curves may, for example, enhance the alignment and/or positioning of the anchor members at a target site. In some variations, the curves may also help to reduce the number of steps to form a tract in tissue, such as eliminating a rotational or grasping step, and generally minimizing the degree to which the tissue is manipulated. This may be especially desirable when forming a tract in fragile tissue. The one or more curves may also help to increase the efficiency of tissue tract formation by helping to ensure consistent tissue contact. Curves in an anchor member may also allow the device to form a tract at various angles; for example, curves may help to form a tract that enters a vessel lumen (e.g., an artery lumen) at a relatively shallow angle (e.g., from about 6° to about 12°, from about 8° to about 10°) or relatively steep angle (e.g., from about 70° to about 90°, from about 75° to about)85°. Some or all of the curves may be in the same plane, or some or all of the curves may be in distinct planes.

For example, and referring now to FIGS. 3E and 3F, anchor member (300) includes two curves that form angles (α₂) and (α₃). More specifically, angle (α₂) is formed by the curve between a distal region (312) and a middle region (314) of anchor member (300), while angle (α₃) is formed by the curve between middle region (314) and a proximal region (316) of anchor member (300). In some variations, angle (α₃) is the same as angle (α₁) in FIG. 2B. Regions (312), (314), and (316) may be integral and/or generally continuous with each other, or at least two of the regions may be coupled to each other and/or generally discontinuous with each other.

FIG. 3E depicts tissue-piercing member (306) exiting delivery guide (308) and crossing anchor member (300). The distance between the location at which tissue-piercing member (306) exits delivery guide (308) and the location at which tissue-piercing member (306) crosses anchor member (300) is crossover length (L_{C1}), which may be, for example, from about 3 millimeters to about 20 millimeters. The crossover length (L_{C1}) may be determined in part by the size (e.g., thickness, length, etc.) of the tissue in which the tract is to be formed, and in some cases, may be larger than the ranges above (e.g., from about 25 millimeters to about 40 millimeters, from about 30 millimeters to about 35 millimeters). Other variations of anchor members may have different crossover lengths, which may affect the shape, length, angle(s), and other characteristics of the tract formed in the tissue.

As shown in FIG. 3F, anchor member (300) is coupled to delivery guide (308) at an angle (α₄), where angle (α₄) is formed by proximal segment (316) and delivery guide (308). Angle (α₂) may be, for example, from about 10° to about 45°, or from about 30° to about 90°, or from about 90° to about 150°, or from about 150° to about 175° (e.g., about 168°); angle (α₃) may be, for example, from about 10° to about 45°, or from about 30° to about 90°, or from about 90° to about 150°, or from about 150° to about 175° (e.g., about 168°); and/or angle (α₄) may be, for example, from about 10° to about 45°, or from about 30° to about 90°, or from about 90° to about 150°, or from about 150° to about 175° (e.g., about 170°).

FIG. 3G depicts the lengths of regions (312), (314), and (316) of anchor member (300). As shown there, region (312) has a length (L₂), region (314) has a length (L₃), and region (316) has a length (L₄). In some variations, one or more of lengths (L₂) and (L₄) may be from about 2 millimeters to about 6 millimeters. In certain variations, length (L₃) may be from about 3 millimeters to about 13 millimeters. Alternatively or additionally, the sum of the three lengths may be from about 7 millimeters to about 25 millimeters. While lengths (L₂), (L₃) and (L₄) are all different from each other, some variations of anchor members may include two or more regions that all have the same length. For example, all of the regions of an anchor member may have the same length. The lengths (L₂), (L₃) and (L₄) may be determined in part by the size (e.g., thickness, length, etc.) of the tissue in which the tract is to be formed, and may be larger than the example ranges above. In some variations, the cross-sectional diameter of anchor member (300) may be from about 0.5 millimeter to about 1.7 millimeters (e.g., about 1.1 millimeters). Again, the diameter of anchor member (300) may vary according to the target tissue.

Referring again to FIG. 3G, delivery guide (308) has a length (L_{D1}) which may be, for example, from about 25 millimeters to about 160 millimeters, and which will be described in additional detail later. The lengths of the individual regions of an anchor member, the overall length of an anchor member, and the crossover length of a tissue-piercing member, may be varied to accommodate the tissue through which the tract is to be formed. In some cases, one or more of these lengths may be adjusted to improve the ability of the device to access the target tissue. For example, the above-described lengths may be partially determined by the size (e.g., thickness, length, etc.) of the tissue in which the tract is to be formed.

Optionally, anchor members may have at least two curves in different planes. For example, FIGS. 3H and 31 depict top views of anchor member (300) (in the case of FIG. 3H, with tissue-piercing member (306)), with FIG. 31 showing that anchor member (300) has additional angles (α₅), (α₆), and (α₇) in its top view. Angle (α₅) is formed by distal region (312) and middle region (314), angle (α₆) is formed by middle region (314) and proximal region (316), and angle (α₇) is formed by proximal region (316) and delivery guide (308). Crossover angle (α_{C1}) is formed by tissue-piercing member (306) and the region of anchor member (300) that is distal to the location at which the tissue-piercing member crosses over. In some variations, angle (α₅) may be from about 10° to about 45°, or from about 30° to about 90°, or from about 90° to about 150°, or from about 135° to about 225° (e.g., about 180°), angle (α₆) may be from about 10° to about 45°, or from about 30° to about 90°, or from about 90° to about 150°, or from about 135° to about 225° (e.g., about 180°), and/or angle (α₇) may be from about 10° to about 45°, or from about 30° to about 90°, or from about 90° to about 150°, or from about 135° to about 225° (e.g., about 180°). In certain variations, crossover angle (α_{C1}) may be from about 10° to about 45°, or from about 30° to about 90°, or from about 90° to about 150°, or from about 2° to about 30°.

As described, anchor member (300) includes angles (α₂)-(α₇), which may reside in one or more distinct planes. For example, angles (α₂)-(α₄) may be in a first plane, while angles (α₅)-(α₇) are in a second plane, where the first and second planes are distinct. In some variations, the planes may intersect. The angles in an anchor member may also occupy more than two distinct planes, for example, 3, 4, 6, or 8 planes. In some variations, each angle may occupy its own distinct plane, separate from the other angles. In certain variations, the distinct planes may intersect with one or more other planes, and/or may be parallel to one another. Distinct planes may have an angle therebetween of about 0° to about 360° (e.g., from about 10° to about 45°, or from about 30° to about 90°, or from about 45° to about 270°, or from about 90° to about 150°, or from about 90° to about 180°. In some variations, anchor member (300) may have one or more non-planar curves, such as curves that form a spiral, which may be approximated by a sufficient number of planar bends. The angles described above may represent planar projections of non-planar curves, which may be useful for inspecting regions of complex geometry. Any of the above-described features (e.g., the number of angles and/or distinct planes, the inclusion or non-planar curves, the intersection of different planes, the angle formed when the tissue-piercing member crosses the anchor, etc.) may be adjusted according to the desired features of the tissue-piercing member deployment and resulting tract.

The lengths of different anchor member regions (e.g., lengths (L₂), (L₃), (L₄), and (L_{D1})), as well as the angles between them, may affect the path of a tissue-piercing member deployed from a delivery guide associated with the anchor member. For example, FIG. 3A shows that when tissue-piercing member (306) is advanced from delivery guide (308), the shape of anchor member (300) causes tissue-piercing member (306) to be deflected to one side of the longitudinal axis of delivery guide (308), as illustrated in FIGS. 3H, 3J, and 3K. More specifically, FIG. 3H is a top view of delivery guide (308), anchor member (300), tissue-piercing member (306), and guide sheath (304), while FIGS. 3J and 3K are bottom and front views, respectively, of the same components (where retainer (302) is visible). These figures show how anchor member (300) deflects tissue-piercing member (306) to one side. This deflection may, in turn, affect the characteristics of the resulting tissue tract. In some variations, lengths (L₂), (L₃), (L₄), and (L_{D1}) may be partially determined by the size (e.g., thickness, length, etc.) of the tissue in which the tract is to be formed.

The crossover length and/or crossover angle of a tissue-piercing member may be adjusted to improve the success rate of tissue tract formation, and may also help determine the characteristics (e.g., size, length, sealing time, etc.) of the resulting tissue tract. In some variations, specific tissue-piercing member paths may be tailored to access tissues with different geometries and thicknesses. Different tissue tracts may provide ready access to one type of tissue, while not providing ready access to a different type of tissue. The tissue-piercing member deployment path that is required to form a tract through a given tissue may be adjusted by altering the angles and/or lengths of the anchor member regions. For example, the angles and lengths of the regions of anchor member (300) cause tissue-piercing member (306) to deflect, as shown in FIG. 3A. Altering the angles and/or lengths of the regions of an anchor member may provide better contact between the anchor member and the tissue, so that a desired tissue tract may be formed with a greater rate of success. Moreover, increasing the contact between the anchor member and tissue may provide enhanced control of the tissue-piercing member, which may help the device to more precisely and consistently maneuver and position the tissue, thereby allowing for the consistent and/or repeatable formation of a desired tissue tract. Other variations of anchor members with different numbers of curves and/or degrees of curvature, and/or with different region lengths (e.g., different lengths (L₂), (L₃), and (L₄), etc.), may provide alternate tissue-piercing member paths, and thus may be used, for example, to form different tracts through different types of tissue. For example, the lengths and angles of an anchor member that may be suitable for forming a tract through an arterial wall may not be suitable for forming a tract through an intestinal wall.

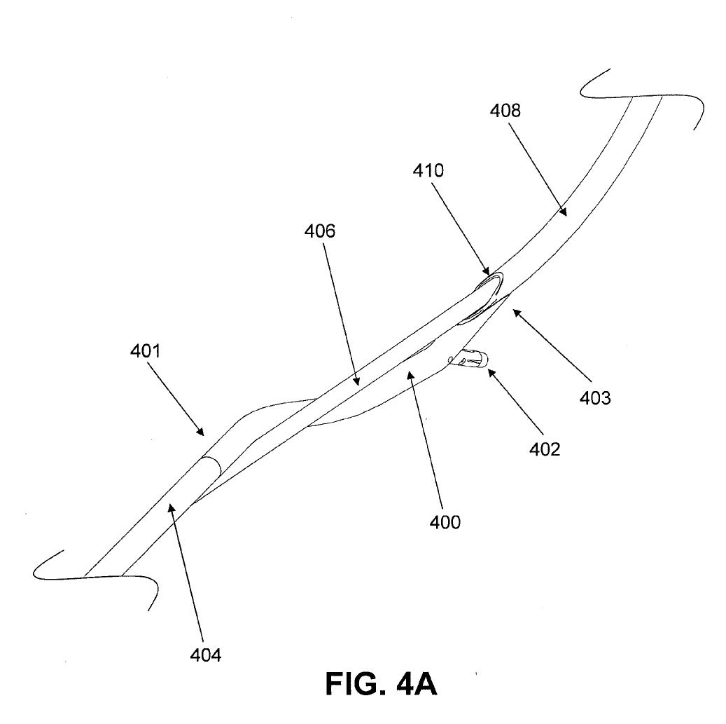

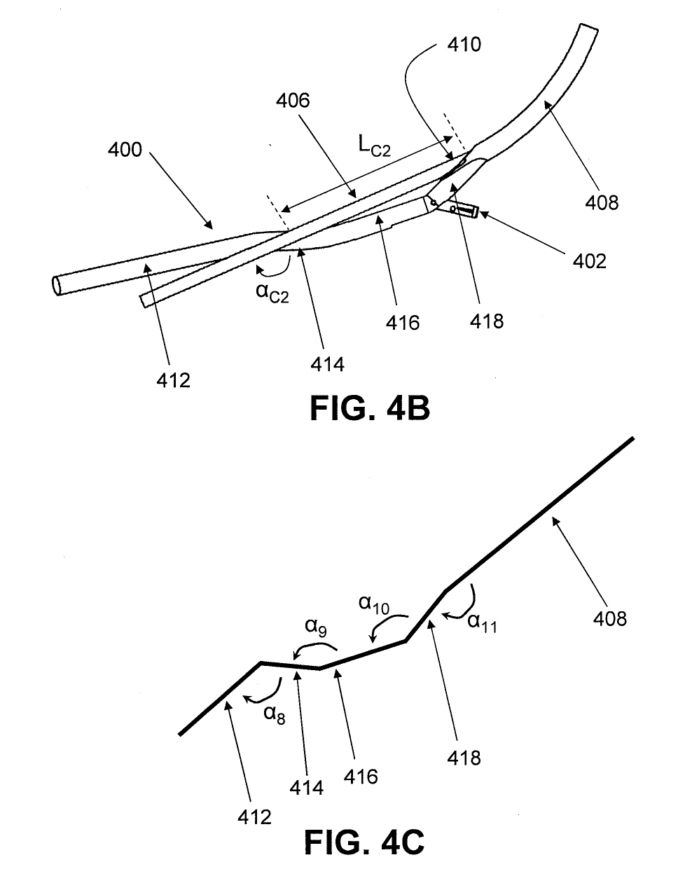

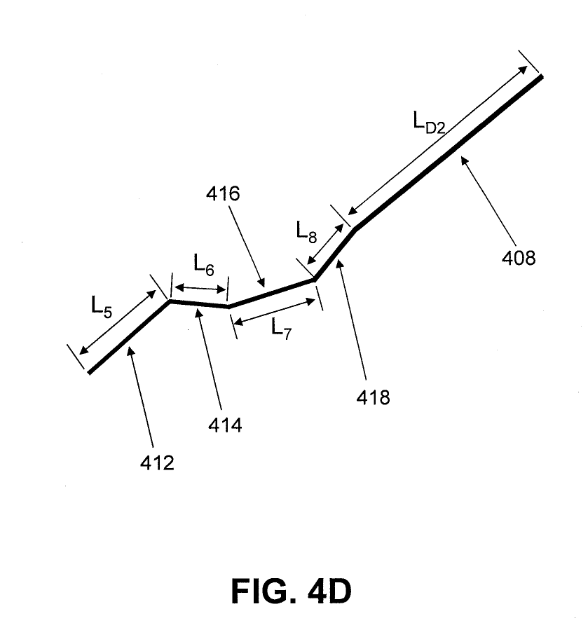

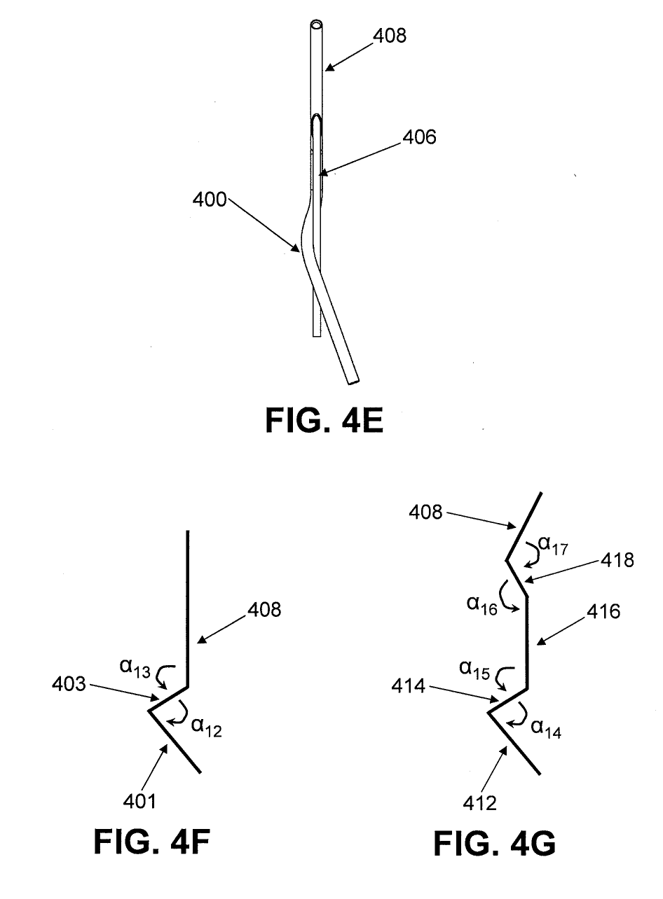

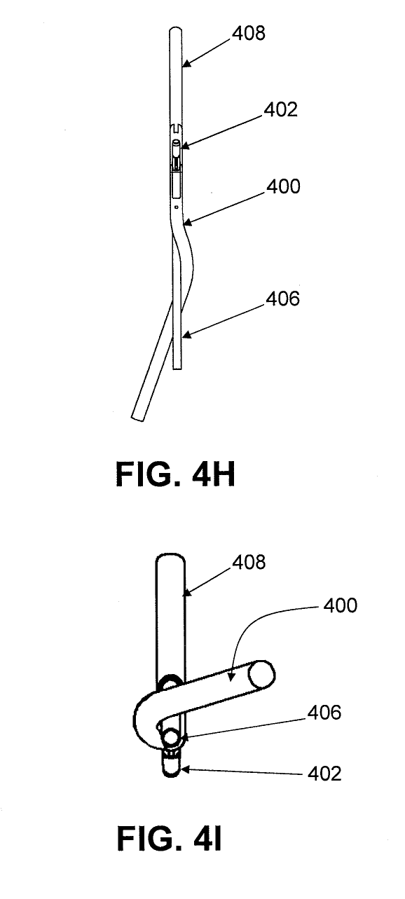

As an example, FIGS. 4A-4I depict another variation of an anchor member (400) that is somewhat corkscrew-shaped. FIGS. 4A and 4B provide perspective and side views, respectively, of anchor member (400) and its associated structures. As shown there, anchor member (400) has a distal portion (401) that is attached to a guide sheath (404), and a proximal portion (403) that is attached to a delivery guide (408). Anchor member (400) comprises different regions having angles therebetween. As shown in FIG. 4B, anchor member (400) includes a distal region (412), a first middle region (414), a second middle region (416), and a proximal region (418). At least some of regions (412), (414), (416), and (418) of anchor member (400) may be integral with each other and/or generally continuous, or may be coupled to each other and/or generally discontinuous. Anchor member (400) also comprises a retainer (402).

Delivery guide (408) comprises a tissue-piercing member port (410), through which a tissue-piercing member (406) may be advanced. For example, FIG. 4B depicts tissue-piercing member (406) exiting delivery guide (408) and crossing anchor member (400). The distance between the location at which tissue-piercing member (406) exits delivery guide (408) and the location at which it crosses anchor member (400) is crossover length (L_{C2}), which may be, for example, from about 3 millimeters to about 20 millimeters. Other variations of anchor members may have different crossover lengths, which may affect the shape, length, and other characteristics of the resulting tissue tract. FIG. 4B also depicts crossover angle (α_{C2}), which is formed by tissue-piercing member (406) and the region of anchor member (400) distal to the location at which tissue-piercing member (406) crosses anchor member (400). Crossover angle (α_{C2}) may vary across different variations of tissue tract-forming devices and may be, for example, from about 20° to about 45°, or from about 30° to about 90°, or from about 90° to about 150°, and may be adjusted to obtain a desired interaction between a tissue-piercing member and an anchor member.

As demonstrated by the figures, anchor member (400) of FIG. 4C is curved, and has three angles (α₈), (α₉), and (α₁₀) between its various regions. More specifically, distal region (412) and first middle region (414) form an angle (α₈), first middle region (414) and a second middle region (416) form an angle (α₉), and second middle region (416) and proximal region (418) form an angle (α₁₀). In some variations, at least one (e.g., all) of angles (α₈), (α₉), and (α₁₀) may be from about 10° to about 45°, or from about 30° to about 90°, or from about 90° to about 150°, or from about 135° to about 175° (e.g., about 168°. At least two of the angles may be different from each other, and/or at least two of the angle may be the same as each other. Additionally, as shown, anchor member (400) may be coupled to delivery guide (408) such that the two components form an angle (a″) therebetween (i.e., between proximal region (418) and delivery guide (408)). In certain variations, angle (α₁₁) may be from about 10° to about 45°, or from about 30° to about 90°, or from about 90° to about 150°, or from about 100° to about 170°.

Regions (412), (414), (416), and (418) may have different lengths, or at least two of the regions may have the same length. In some variations, all of the regions of an anchor member having multiple regions may have the same length. As shown in FIG. 4D, distal region (412) has a length (L₅), first middle region (414) has a length (L₆), second middle region (416) has a length (L₇), and proximal region (418) has a length (L₈). In certain variations, at least one of lengths (L₅), (L₆), (L₇), and/or (L₈) may be from about 2 millimeters to about 6 millimeters. Additionally, and referring again to FIG. 4D, delivery guide (408) has a length (L_{D2}) which may be, for example, from about 25 millimeters to about 100 millimeters. For example, the above-described lengths may be partially determined by the size (e.g., thickness, length, etc.) of the tissue in which the tract is to be formed. The diameter of anchor member (400) may be, for example, from about 0.5 millimeter to about 1.7 millimeters (e.g., about 1.1 millimeters). Again, the diameter of the anchor member (400) may vary according to the target tissue.

Optionally, anchor members may have one or more curves in a second plane that is distinct from a first curvature plane of the anchor member, as described above and as shown here with reference to FIGS. 4E-4G. Referring specifically now to FIGS. 4E and 4F, anchor member (400) has additional angles (α₁₂) and (α₁₃) in a second plane that is generally orthogonal to the plane including angles (α₈), (α₉), (α₁₀), and (α₁₁). Angle (α₁₂) is formed by distal portion (401) and proximal portion (403), and may be, for example, from about 3° to about 30°, or from about 10° to about 45°, or from about 30° to about 90°, or from about 90° to about 150°, or from about 60° to about 150°. Angle (α₁₃) is formed by proximal portion (403) and delivery guide (408), and may be, for example, from about 3° to about 30°, or from about 10° to about 45°, or from about 30° to about 90°, or from about 90°to about 150°, or from about 45° to about 150°. In some variations, anchor member (400) may have one or more non-planar curves, which may be approximated by a sufficient number of planar bends. The angles described above may represent planar projections of non-planar curves, which may be useful for inspecting regions of complex geometry.

Alternatively or additionally, anchor member (400) may have angles (α₁₄), (α₁₅), (α₁₆), and (α₁₇) in an additional plane, where the angles may generally form a corkscrew arrangement, as shown in FIGS. 4G and 41. Angle (α₁₄) is formed by distal region (412) and first middle region (414), angle (α₁₅) is formed by first middle region (414) and second middle region (416), and angle (α₁₆) is formed by second middle region (416) and proximal region (418). Finally, angle (α₁₇) is formed by proximal region (418) and delivery guide (408). In some variations, angle (α₁₄) may be from about 3° to about 30°, or from about 10° to about 45°, or from about 30° to about 90°, or from about 90° to about 150°, or from about 60° to about 150°; angle (α₁₅) may be from about 3° to about 30°, or from about 10° to about 45°, or from about 30° to about 90°, or from about 90° to about 150°, or from about 45° to about 150°; angle (α₁₆) may be from about 3° to about 30°, or from about 10° to about 45°, or from about 30° to about 90°, or from about 90° to about 150°, or from about 45° to about 150°; and/or angle (α₁₇) from about 3° to about 30°, or from about 10° to about 45°, or from about 30° to about 90°, or from about 90° to about 150°, or from about 30° to about 170°. For example, in certain variations, angle (α₁₄) may be about 50°, (α₁₅) may be about 120°, (α₁₆) may be about 150°, and/or (α₁₇) may be about 120°. In some variations, the angles described above may represent planar projections of non-planar curves.

These angles may be selected such that at least a portion of the anchor member (400) wraps around one or more portions of the tissue-piercing member (406), as evident by the top, bottom, and front views of the device shown in FIGS. 4E, 4H, and 41, respectively. Angles in multiple distinct planes may position the tissue to guide the path of tissue-piercing member (406) as it is advanced from delivery guide (408). For example, in FIG. 41, tissue-piercing member (406) is surrounded above, below, and on one side by anchor member (400), and may help to position the tissue with respect to the tissue-piercing member. Anchor member (400) may also help to ensure that the tissue-piercing member is not deflected as it penetrates the tissue.

As described, the anchor member (400) depicted in FIGS. 4C, 4F, and 4G has angles (α₈)-(α₁₇), which may be located in one or more distinct planes. For example, angles (α₈)-(α₁₁) may be in a first plane, and angles (α₁₂)-(α₁₃) and/or angles (α₁₄)-(α₁₇) may be in a second plane, where the first and second planes are distinct, and in some variations, intersect each other. Angles (α₈)-(α₁₇) may also occupy more than two distinct planes, for example, 3, 4, 6, or 8 planes. For instance, each angle may occupy its own distinct plane, separate from the other angles. Crossover angle (α_{C2}) may be in a distinct plane from the other angles described above, or may be co-planar with one or more angles. In some variations, the distinct planes may intersect one or more other planes, and/or may be parallel to one another. Distinct planes may intersect at an angle of about 0° to about 360° (e.g., from about 45° to about 270°, from about 90° to about 180°). In some variations, anchor member (400) may have one or more non-planar curves which are not constrained in a plane, which may be approximated by a sufficient number of planar bends. The number of angles and distinct planes, as well as the intersection of planes, may be adjusted according to the desired degree of constraint of the tissue-piercing member, and as well as to achieve a specific tissue tract in the target tissue (e.g., arterial wall).

Angles (α₈)-(α₁₇) and lengths (L₅)-(L₈) and (L_{D2}) of regions (412), (414), (416), and (418) of anchor member (400) may shape the path of deployment of tissue-piercing member (406) from delivery guide (408). As such, the characteristics of the tissue tract formed by tissue-piercing member (406) may be determined to some extent by the features of anchor member (400). For example, the angle of a tissue tract through a vessel wall as it enters the vessel lumen may be relatively shallow (e.g., from about 6° to about 12°, from about 8° to about 10°) or relatively steep (e.g., from about 60° to about 90°, from about 70° to about 80°), which may be determined in part by the dimensions (e.g., angles and lengths) of the anchor member. The angles and lengths of the components of the anchor member may also affect the degree to which the tissue is manipulated as the tract is formed, which in turn may affect the rate at which the tract self-seals upon removal of the tract-forming device. Moreover, tissue-piercing member (406) first passes superior to anchor member (400), and then passes inferior to anchor member (400). This may help to direct the path of tissue-piercing member (406) somewhat during deployment, thereby reducing unintended deviations by tissue-piercing member (406). As a result, the tissue-piercing member may be advanced in a relatively precise, predictable, and/or repeatable manner.

Of course, anchor member (400) is only one variation of an anchor member, and other variations of anchor members may be used in tissue-tract forming devices. The configuration of any particular anchor member may be selected, for example, to help guide or stabilize one or more tissue-piercing members in a particular way during their deployment. As an example, an anchor member may be configured to help achieve a particular tissue-piercing member deployment path through tissue having a specific geometry and/or thickness. For example, the anchor member may have a certain number of angles in a first plane, and/or another number of angles in a second plane, and/or may include angles of different sizes from those shown above. In some cases, the number of planes and/or angles defining an anchor member's geometry may be increased to reduce the possibility that the tissue-piercing member will “skip off” of the target tissue (e.g., the tissue of an arterial wall), rather than penetrating its surface.

Additional angles in distinct planes may also reduce or eliminate the amount of manual adjustment of the device (e.g., tilting, etc.) that may be necessary to form a path through a particular tissue. For example, an anchor member may have multiple angles and turns in the shape of a helix that helps to direct a tissue-piercing member along its central axis. In certain variations, the lengths of different segments of an anchor member may be altered to change the resulting tissue-piercing member path through a given tissue. Varying such characteristics of an anchor member may allow for different approaches of the tissue-piercing member through tissue. For example, the characteristics of an anchor member may allow a tissue tract to be formed with a relatively shallow angle (e.g., from about 6° to about 12°, from about 8° to about 10°) or a relatively steep angle (e.g., from about 60° to about 90°, from about 70° to about 80°). In certain variations, the anchor member may be sized and shaped to help form a tract in tissue of a certain elasticity or toughness. This may be important, for example, if one approach does not provide ready access to a particular target site in a tissue, while another approach does provide ready access to the target site. For example, different approaches (e.g., tissue tracts of different angles and lengths) may be necessary to access tissues of different geometries (e.g., a relatively cylindrical artery vs. a relatively elliptical stomach).

Anchor member (400) as shown in FIGS. 4A-4I may, for example, help tissue-piercing member (406) follow a prescribed access pathway through tissue, such as a vessel wall (e.g., an arterial wall). Other variations of anchor members with different numbers of curves and/or degrees of curvature may provide alternate tissue-piercing member paths (e.g., that may be used to form different tracts through different types of tissue). In variations where an anchor member contacts fluid flow (e.g., blood flow in an artery), the anchor member may be stream-lined and/or shaped to reduce flow obstruction, for example.

Anchor members may be formed from a single material, or multiple materials. In some variations, an anchor member may comprise one or more materials that allow for firm contact with the tissue through which a tract is to be formed. For example, anchor members may comprise one or more metal alloys (e.g., stainless steel, nickel titanium alloy, etc.) and/or one or more polymers (e.g., carbon-filled, thermoplastic polymers, thermoset plastics, epoxy resins, etc.). Moreover, in some variations, an anchor member may be surface-modified so that the anchor member is rougher on its surface and/or otherwise more likely to engage a tissue. Surface modification may also result in enhanced visibility under ultrasound.

In certain variations, an anchor member may comprise a machined hypotube. In some variations, an anchor member may be formed by assembling two or more components formed by Swiss screw machining, or may be integrally formed by Swiss screw machining. Alternatively or additionally, an anchor member may be formed by assembling two or more components using mechanical junctions, form-fitting, screw-fitting, snap-fitting, adhesive-bonding, brazing, soldering, welding, heat-bonding, and the like. In certain variations, at least a portion of an anchor member may be hollow. For example, an anchor member may comprise one or more lumens (e.g., for use in delivery of one or more therapeutic agents and/or a saline flush). In variations in which an anchor member comprises one or more curves, the curves may be formed, for example, when the main body of the anchor member is formed, or after the main body has been formed. For example, curves may be introduced into an anchor member by deflecting, heating, melting, bending, forging, and/or molding one or more portions of the anchor member.

In some variations, and as discussed briefly above, an anchor member may include one or more surface modifications (e.g., to enhance the contact between the anchor member and the target tissue). For example, an anchor member may comprise one or more grooves, ridges, slots, and/protrusions, and/or any surface coating or coatings that modify the anchor member's frictional interactions with tissue (i.e., increase or decrease friction, as appropriate).

In some cases, an anchor member may include one or more slots and/or other apertures. These apertures may, for example, allow for the storage and release of one or more therapeutic agents from the anchor member. Alternatively or additionally, they may allow for a certain degree of flexibility and maneuverability. Optionally, one or more portions of an anchor member may have one or more lumens therethrough, while other anchor members may be substantially solid.

The proximal portion of an anchor member may be coupled to a delivery guide (see, e.g., FIGS. 3A and 4A) using any appropriate technique. For example, in some variations, metal or metal alloy anchor members and delivery guides may be welded together, form-fit, screw-fit, snap-fit, brazed, soldered, bonded by one or more adhesives, and the like. An anchor member and a delivery guide may also be mechanically coupled to each other (e.g., using hinges, etc.). In certain variations, an anchor member and a delivery guide may be integral with each other, and thus may not require any additional features for coupling purposes.

Any appropriate type of tissue-piercing member may be used with the devices and methods described here, and in some variations, multiple tissue-piercing members may be used (e.g., a device may be capable of deploying two different tissue-piercing members). In some variations, a tissue-piercing member may have one or more lumens therethrough for the delivery of various devices and/or therapeutic agents. In certain variations, there may be openings, slits, or ports at the distal end of the tissue-piercing member sized and shaped for the delivery of therapeutic agents. For example, the tissue-piercing member may be in the form of a cannula with a distal end configured to pierce tissue. Alternatively, a substantially solid tissue-piercing member may be used, and may provide a relatively small puncture. For example, the tissue-piercing member may be a lancet. The sharpened distal portion of a tissue-piercing member may have one or more sharp edges, and/or may have a single sharp point at the distal-most tip. The sharpened distal portion may be beveled, or may be substantially straight. The geometry and size of the sharpened distal portion may be chosen based on the geometry and size of the tissue tract to be formed. In some variations, the tissue-piercing member may comprise a hypotube formed of a biocompatible material, such as a stainless steel hypotube. The tissue-piercing member may be substantially straight, or may have one or more curves, as appropriate to obtain the desired tissue tract.

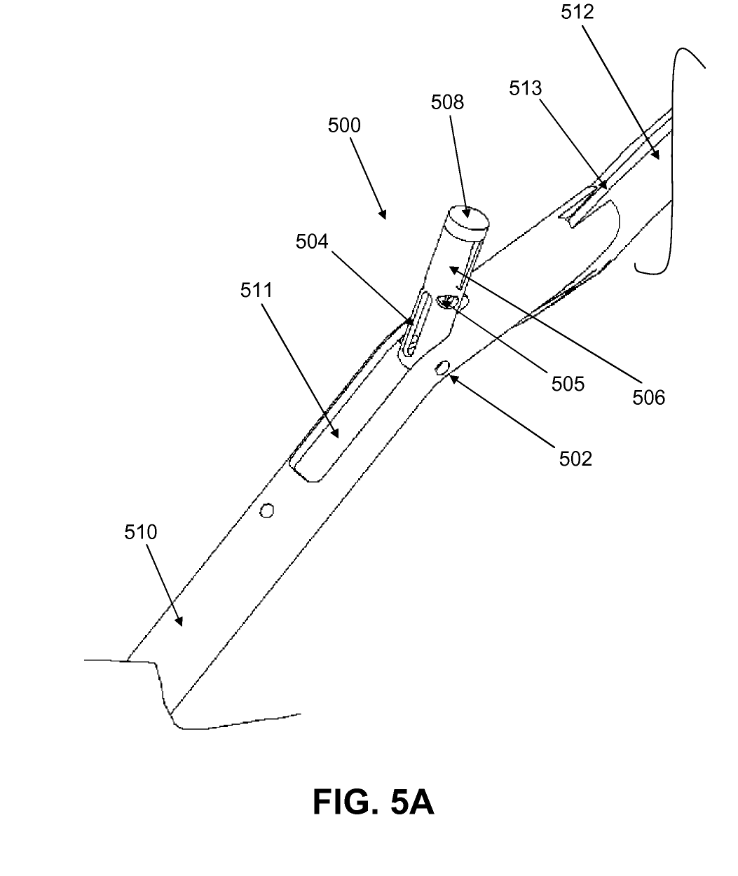

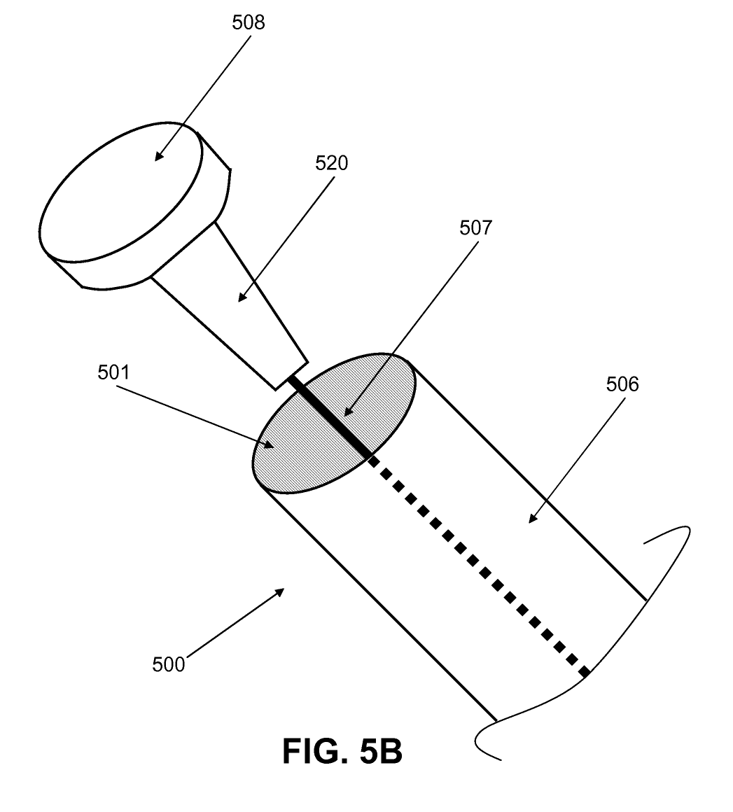

Tissue tract-forming devices may include one or more retainers that may be used, for example, to help accurately position the devices at a target site. For example, FIGS. 3A and 4A depict variations of anchor members comprising retainers (302) and (402), respectively. Additionally, FIGS. 5A and 5B depict one variation of a retainer (500) in enhanced detail. As shown there, retainer (500) comprises a retainer body (506), a coupling feature (502), and apertures (504) and (505). Additionally, retainer (500) is configured to be articulated into and out of a slot (511) in an anchor member (510). In the variation shown, slot (511) is sized and shaped to match the size and shape of the retainer. Coupling feature (502) may, for example, comprise a hinge that acts as a pivoting point, such that retainer (500) can rotate into and out of slot (511). Alternatively or additionally, one or more other coupling features may be used that allow the retainer to be actuated with additional degrees of freedom. Non-limiting examples of such coupling features include slide bars that permit the retainer to be moved laterally along slot (511), ball hinges that allow the retainer to be rotated into and out of slot (511) and to rotate axially, and the like. Coupling feature (502) may be made of any appropriate material or materials including, for example, stainless steel or nickel titanium alloys (e.g., Nitinol). Retainer body (506) may comprise any appropriate material or materials, and in some cases, may be formed from a stainless steel hypotube. In certain variations, retainer body (506) may have a lumen therethrough that houses a cable, described in detail below.

Retainer (500) may be actuated in any of a number of different ways. FIG. 5B depicts one variation of an actuation mechanism that may be used to direct the movement of retainer (500). As shown there, retainer body (506) includes a lumen (501) therethrough, which houses a cable (507). Cable (507) extends from a cable tip (508), through retainer body (506), and into a distal portion of a delivery guide or an actuator lumen (not shown in FIG. 5B). Cable tip (508) may have a tapered body (520), which may facilitate the coupling between the cable tip and the retainer body. The tapered body may help to keep the tip engaged in the retainer body when the cable is slack. In some variations, the cable tip may be a ball. Cable (507) may be actuated (e.g., tensioned or released), for example, at the proximal portion of a delivery guide, which in turn may direct the movement of retainer (500). For example, cable (507) may be coupled to a lever of the handle, as shown later in FIGS. 8M and 8N.

Cable tip (508) may be sized such that its diameter is greater than the diameter of lumen (501). As a result, the lumen (501) may act as a stop for cable tip (508). Cable tip (508) may be formed, for example, from one or more metals, metal alloys (e.g., stainless steel), high strength polymers, and/or any other appropriate materials. In some variations, a cable tip may be in the form of a ball that is formed, for example, by melting the cable tip material or materials.

Cable (507) may comprise any appropriate material or materials, such as one or more metals, metal alloys (e.g., stainless steel), polymers (e.g., ultra-high molecular weight polyethylene (UHMWPE) or Aramid aromatic polyamide fibers), and/or spin-extruded materials (e.g., spin-extruded UHMWPE, such as SPECTRA spin-extruded UHMWPE). In some cases, a cable such as cable (507) may be formed by extrusion. Alternatively or additionally, a cable may be formed by weaving a plurality of individual strands together. In certain variations, one or more polymers (e.g., high strength polymers) may be molded over a cable.

Cable (507) typically may be fixedly coupled to cable tip (508) (e.g., using welding, adhesive-bonding, crimping, etc.). When cable (507) is tensioned, cable tip (508) may be pulled toward retainer body (506). Cable tip (508) may be drawn into lumen (507) until the cable tip stops against the lumen, since the tip diameter is greater than the lumen diameter. Further tensioning may apply a force that pivots retainer (500) around coupling feature (502), thereby pulling the retainer entirely out of slot (511), and into the position shown in FIG. 5A. Releasing the tension on cable (507) may allow cable tip (508) to fall away from retainer body (506), similar to what is shown in FIG. 5B, and may also allow retainer (500) to pivot toward slot (511). When the retainer is within the slot (511) of anchor (510), i.e. a parked position, the extension of the cable (507) due to the release of tension may act to hold the retainer in slot (511). In the parked position, the reduced cable tension may allow the cable tip (508) to extend away from the retainer body (506), which may catch on the inner edge of slot (511), while maintaining a portion of tapered body (520) within lumen (501), thus maintaining the retainer in the slot. Maintaining a parked position may contribute to a smaller anchor member profile which may, in turn, result in a reduced likelihood of tissue damage by the anchor member during use. Other mechanisms of actuating retainer (500) may also be used to coordinate retainer movement with the general operation of the tissue tract-forming device.

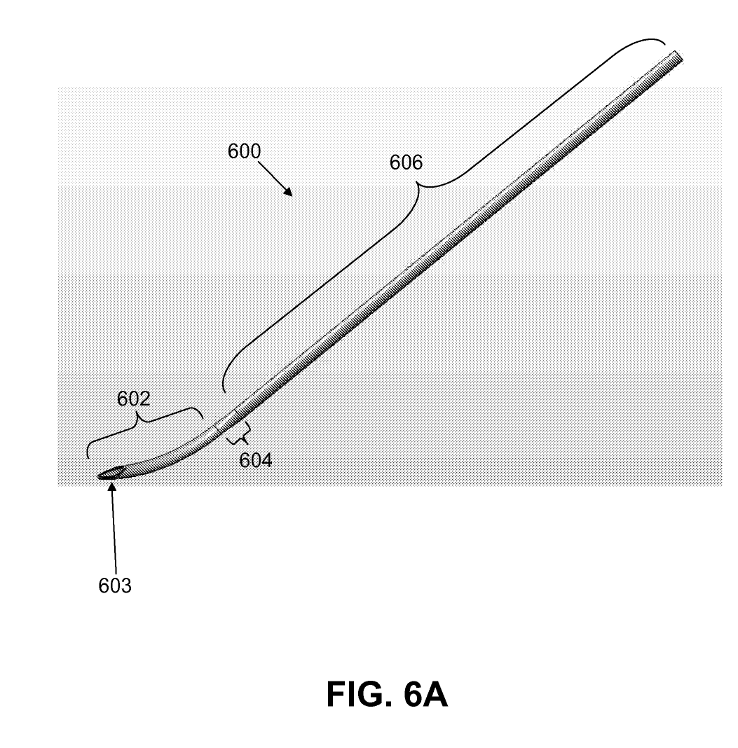

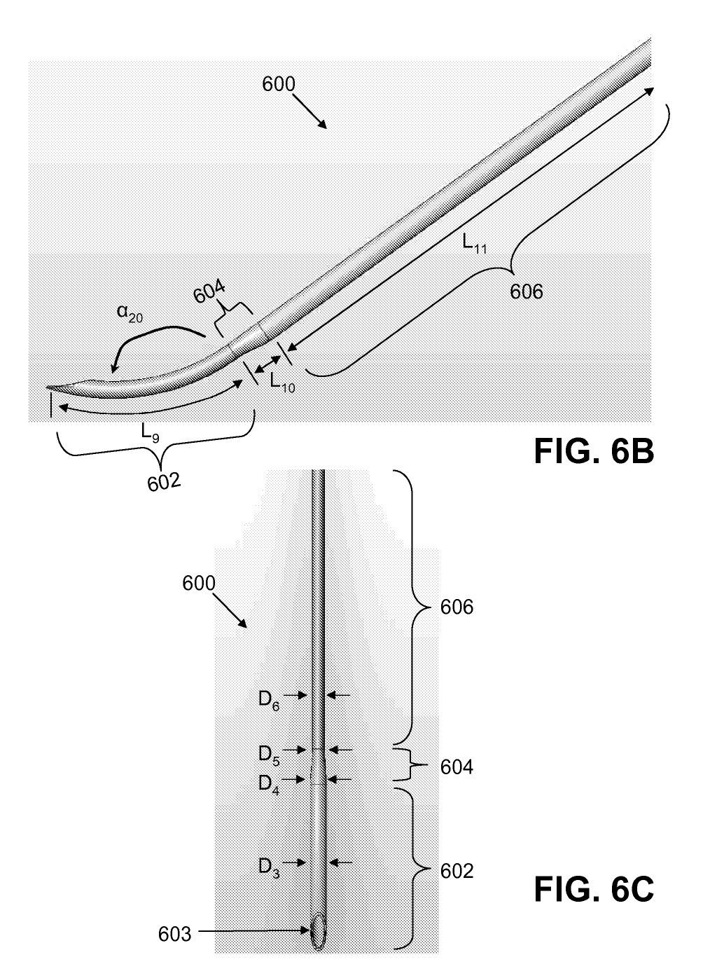

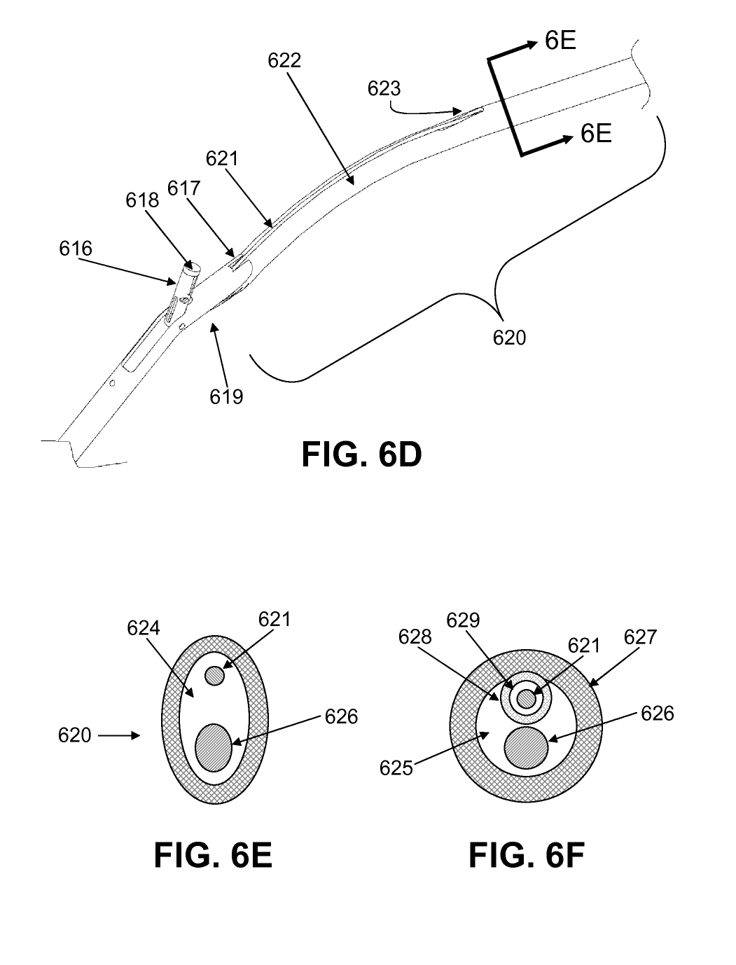

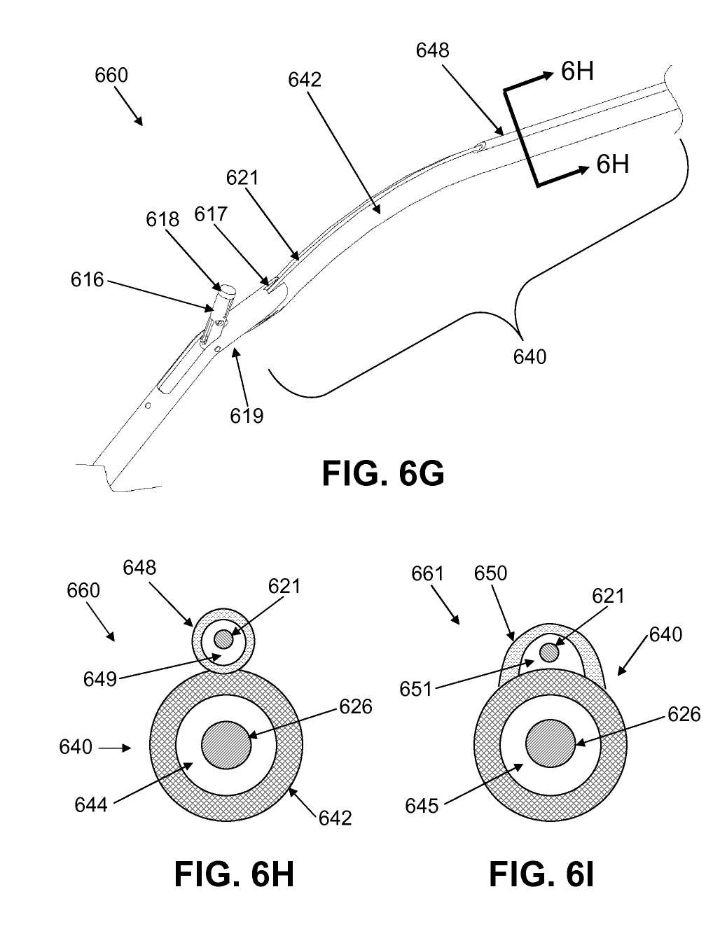

As described previously, the proximal portion of an anchor member may be coupled to a delivery guide. One variation of a delivery guide (600) is shown in FIGS. 6A-6C. First, FIG. 6A provides a perspective view of delivery guide (600). As shown there, delivery guide (600) comprises a distal portion (602), a neck (604), and a shaft (606). Delivery guide (600) also has a longitudinal lumen therethrough (not shown), which is in fluid communication with a tissue-piercing member port (603) at the distal end of distal portion (602). Some variations of a delivery guide may also comprise a side port in the proximal portion of shaft (606), where the side port may be sized and shaped for redirecting fluid (e.g., blood, interstitial fluid, etc.) to outside of the delivery guide. The side port may be a hole or slit. A tissue-piercing member (not shown) may be housed in the lumen of delivery guide (600), and may be controllably advanced through tissue-piercing member port (603).

At least two or all of distal portion (602), neck (604), and shaft (606) may be integral with each other, or at least two or all of them may be individually formed and then coupled to each other. Distal portion (602), neck (604), and/or shaft (606) may be made of the same material or materials, or at least one of them may be made of different material(s) from the others. In some variations, distal portion (602), neck (604), and/or shaft (606) may comprise different materials with different physical and structural properties (e.g., flexibility, opacity, durability, etc.), as appropriate to the function of each part. For example, distal portion (602) and shaft (606) may comprise a relatively rigid material (e.g., stainless steel), while neck portion (604) may comprise a relatively flexible material (e.g., silicone). Alternatively, distal portion (602), neck (604), and shaft (606) may all be made of the same materials(s) (e.g., stainless steel), and/or may all be relatively rigid or flexible. Additionally, in some variations, neck portion (604) and/or shaft (606) may comprise one or more features (e.g., slits) to permit a certain degree of flexibility.

FIGS. 6B and 6C depict side and top views, respectively, of delivery guide (600). As shown there, distal portion (602) has a length (L₉) and a dimension (D₃) (e.g., a cross-sectional diameter). In some variations, length (L₉) may be from about 5 millimeters to about 25 millimeters, and/or dimension (D₃) may be from about 0.7 millimeter to about 3 millimeters (e.g., from about 1.5 millimeters to about 2 millimeters). Neck (604) has a length (L₁₀), which may be, for example, from about 1 millimeter to about 5 millimeters (e.g., from about 2 millimeters to about 4 millimeters). In the variation shown in FIGS. 6A-6C, neck (604) tapers from one thickness distally to a second thickness proximally. More specifically, and as shown in FIG. 6C, the proximal portion of neck (604) has a dimension (D₅) (e.g., a cross-sectional diameter), while the distal portion of neck (604) has a dimension (D₄) (e.g., a cross-sectional diameter). In some variations, dimension (D₅) may be from about 0.5 millimeter to about 2 millimeters (e.g., from about 1 millimeter to about 1.5 millimeters), and/or dimension (D₄) may be from about 0.7 millimeter to about 3 millimeters (e.g., from about 1 millimeter to about 2 millimeters).

The above-described lengths may be partially determined by the size (e.g., thickness, length, etc.) of the tissue in which the tract is to be formed, and may, for example, be larger or smaller than the exemplary dimensions above. In certain variations, dimension (D₅) may match the thickness of shaft (606). Of course, while not shown here, necks having other configurations may be used, as appropriate. For example, in some variations, a delivery guide may comprise a neck having a uniform thickness, and/or a neck having a different thickness from a distal portion and/or shaft of the delivery guide. Additionally, a delivery guide may comprise more than one tapered portion, as appropriate.

Referring again to FIGS. 6B and 6C, shaft (606) has a length (L₁₁), which may be, for example, from about 25 millimeters to about 100 millimeters (e.g., from about 50 millimeters to about 75 millimeters), and a dimension (D₆) (e.g., a cross-sectional diameter), which may be, for example, from about 0.5 millimeter to about 3 millimeters (e.g., from about 1 millimeter to about 2 millimeters). While not shown here, the distal portion and/or shaft of a delivery guide may have more than one thickness in other variations. Moreover, in some variations, a delivery guide may comprise a different number or arrangement of portions, or may not even comprise multiple different portions.

Distal portion (602) of delivery guide (600) has a pre-shaped curve, where the angle of curvature is (α₂₀) (FIG. 6B). In some variations, angle (α₂₀) may be from about 10° to about 45°, or from about 30° to about 90°, or from about 90° to about 150°, or from about 15° to about 60°. Angle (α₂₀) may be adjusted, for example, to achieve a desired deployment of a tissue-piercing member at a target tissue, and may be steep, moderate, or shallow. In some variations, a delivery guide may comprise a distal portion having more than one pre-shaped curve, in one or more planes. Alternatively or additionally, a delivery guide may comprise a neck and/or shaft having one or more pre-shaped curves in one or more planes. The curves may, for example, facilitate the formation of one or more tracts through tissue. In certain variations, the angles of curvature of the curves in a delivery guide may be adjusted (e.g., as the tissue tract-forming device is in use). For example, the delivery guide may comprise one or more members that may be used to deflect one or more portions of the delivery guide. In some variations, a delivery guide may comprise a portion (e.g., a distal portion) comprising one or more relatively flexible materials, such that the portion is capable of curving and conforming to tissue during use.

Delivery guide (600) comprises a lumen therethrough (not shown). A tissue-piercing member (also not shown) is housed within the lumen, and may exit at the distal portion of the delivery guide, through a tissue-piercing member port (603). While delivery guide (600) is depicted as having just one tissue-piercing member port (603), some variations of delivery guides may have multiple tissue-piercing member ports, such as 2, 3, 4, or 5 tissue-piercing member ports. This may, for example, allow for tissue-piercing members to be deployed in different locations, or allow for a tailored deployment location for a particular tissue-piercing member.

In certain variations, an actuating cable also may be at least partially housed within a lumen of a delivery guide. For example, FIGS. 6D and 6E depict one variation of a tissue-tract forming device (660) comprising a delivery guide (620) and an actuating cable (621). The distal end of actuating cable (621) is coupled to a tip portion (618) of a retainer (616) extending from an anchor member (619) of device (660). Actuating cable (621) passes through anchor member (619), and exits the anchor member via a slit (617), at which point actuating cable (621) traverses along the exterior of a delivery guide shaft (622) of delivery guide (600). Actuating cable (621) then enters a lumen (624) of delivery guide shaft (622) via an aperture (623), and passes through the lumen until it reaches a proximal portion of the device (e.g., an actuating handle), where the tension on the actuating cable may be adjusted.

FIG. 6E provides a cross-sectional view of delivery guide (620), showing both actuating cable (621) and a tissue-piercing member (626) housed within lumen (624), which is elliptically shaped. The elliptical shape of lumen (624) may, for example, help the lumen to accommodate both the actuating cable and the tissue-piercing member without resulting in substantial contact or interference between them during use. However, while an elliptical shape is shown here, other variations of delivery guides may have different appropriate cross-sectional shapes. Moreover, while one lumen (624) is shown as housing both actuating cable (621) and tissue-piercing member (626), some variations of devices may comprise two or more lumens, with an actuating cable and a tissue-piercing member each disposed in a different lumen. For example, FIG. 6F shows a delivery guide (627) comprising a lumen (625) and a tubular member (628) disposed within the lumen and having its own lumen (629). Tubular member (628) may, for example, be secured to a wall of lumen (625), or may be free to move within lumen (625). Housing an actuating cable and a tissue-piercing member in separate lumens may, for example, ensure that there is no unintentional contact between the actuating cable and the tissue-piercing member. For example, isolating an actuating cable from a tissue-piercing member may prevent accidental severing of the actuating cable by the tissue-piercing member. Alternatively, housing both an actuating cable and a tissue-piercing member within a common lumen may allow for a relatively simple delivery guide design.

Lumens (624), (625), and (629) may be of any appropriate size and shape, which may depend, for example, on the size and shape of actuating cable (621) and/or tissue-piercing member (626). It should be noted that while certain structures for housing actuating cables and tissue-piercing members have been described, other structures may be used, as appropriate.

A tissue-piercing member, such as tissue-piercing member (626), may have any suitable configuration or shape. As an example, a tissue-piercing member may have an elliptical cross-sectional shape, as depicted in FIG. 6E, or a substantially circular cross-sectional shape, as depicted in FIG. 6F, or may have any other appropriate shape. Moreover, the shape of a tissue-piercing member need not necessarily match the shape of a lumen in which it is disposed. For example, a tissue-piercing member that is disposed within a lumen having an elliptical cross-section may itself have a circular cross-section. Generally, tissue-piercing members have a distal tip that is suitable for piercing or cutting tissue (e.g., sharpened, beveled, pointed, etc.). Tissue-piercing members may be solid (as with the tissue-piercing members depicted in FIGS. 6E and 6F), or in some variations, at least a portion of a tissue-piercing member may have one or more lumens therethrough. A tissue-piercing member may be shaped with one or more curves which may, for example, match the curvature of a delivery guide in which the tissue-piercing member is disposed. Alternatively, a tissue-piercing member may be substantially straight (i.e., having an angle of curvature of about 180°). In some variations, a tissue-piercing member may be coupled to an actuating mechanism (e.g., at its proximal end), such as a handle or a pushing member (e.g., a plunger).

Another variation of a delivery guide is depicted in FIGS. 6G and 6H. As shown there, a tissue tract-forming device (660) comprises a delivery guide (640) including a lumen (644) therethrough, and a tissue-piercing member (626) disposed within lumen (644). Tissue tract-forming device (660) also includes actuating cable (621) extending from a cable tip (618) and through an anchor member (619) of tissue tract-forming device (660). Actuating cable (621) exits anchor member (619) via a slit (617), and traverses the exterior of a shaft (642) of delivery guide (640), before entering an actuating lumen (649) of a tubular member (648). Tubular member (648) is coupled (e.g., welded) to the exterior surface of shaft (642) of delivery guide (640), and may be formed, for example, from a hypotube, and/or may comprise one or more metals and/or metal alloys, and/or any other suitable materials. As shown, tissue-piercing member (626) is housed within lumen (644) of shaft (642).

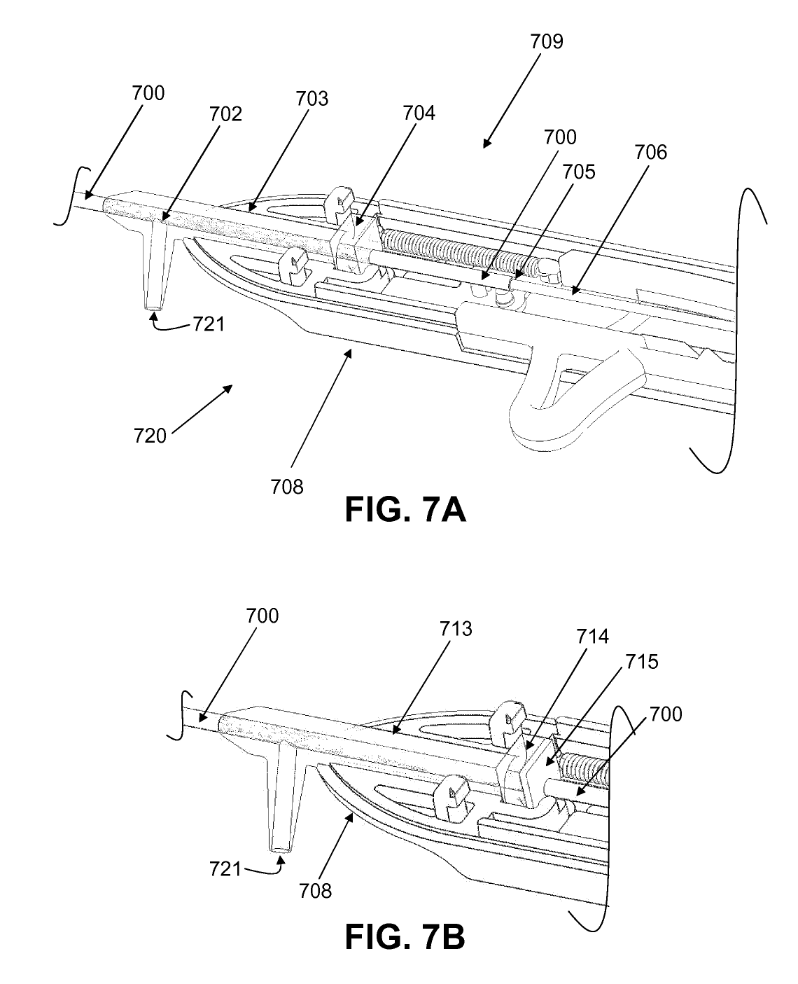

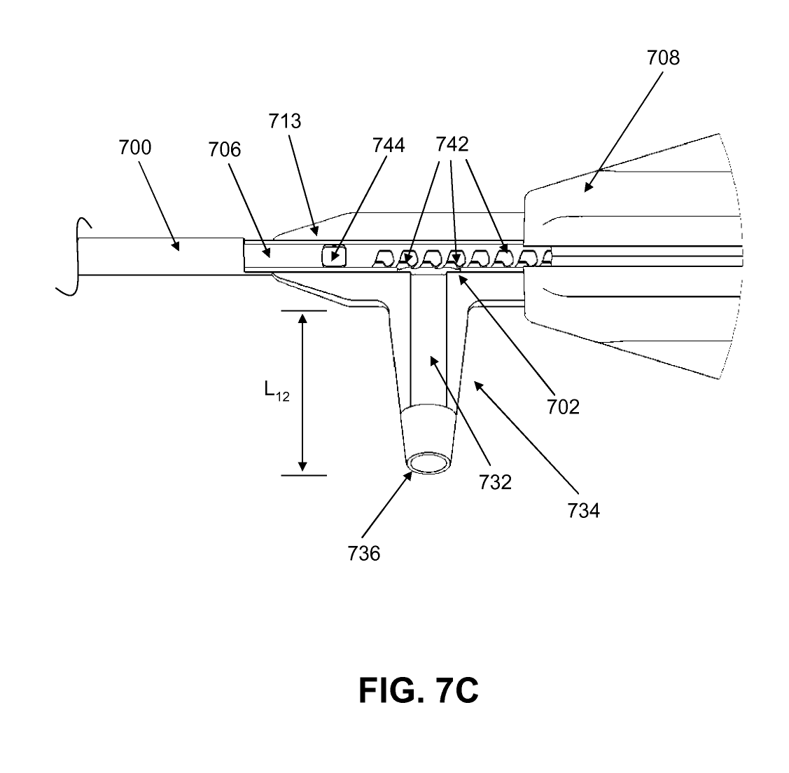

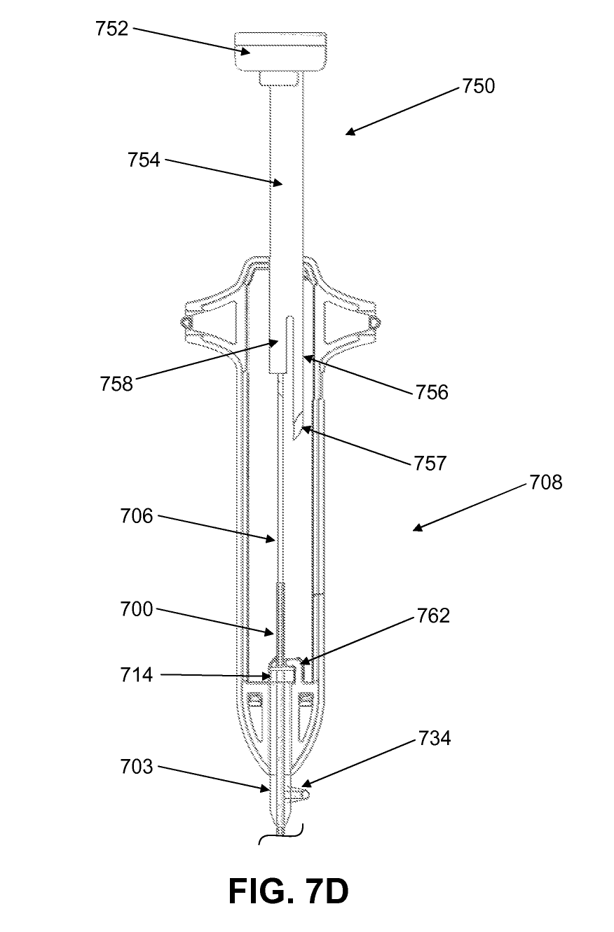

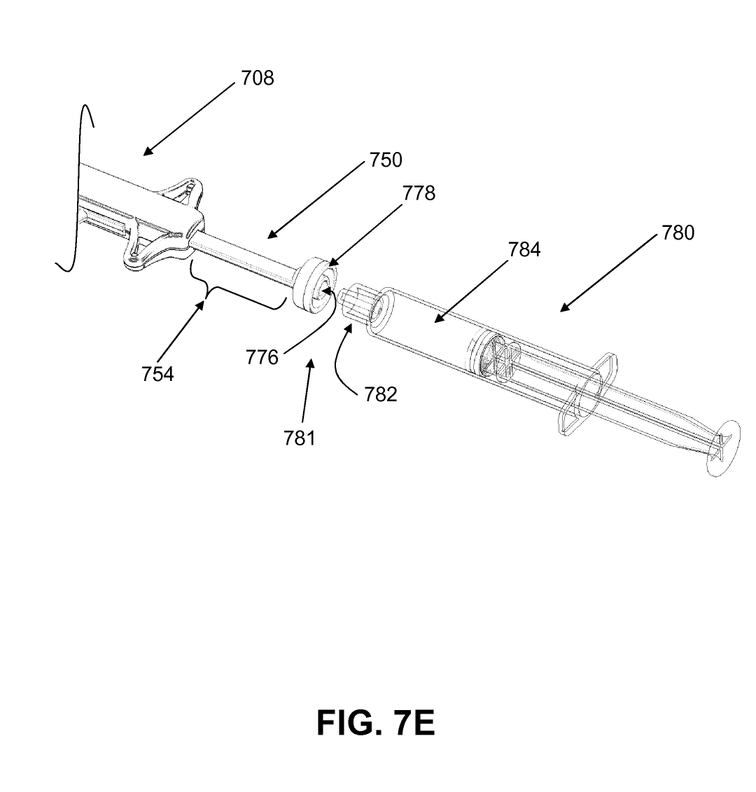

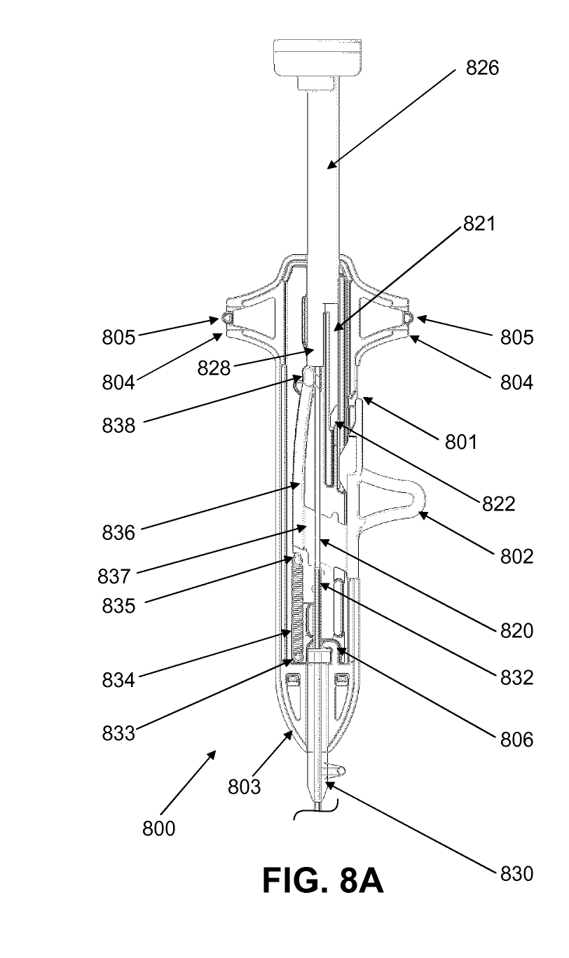

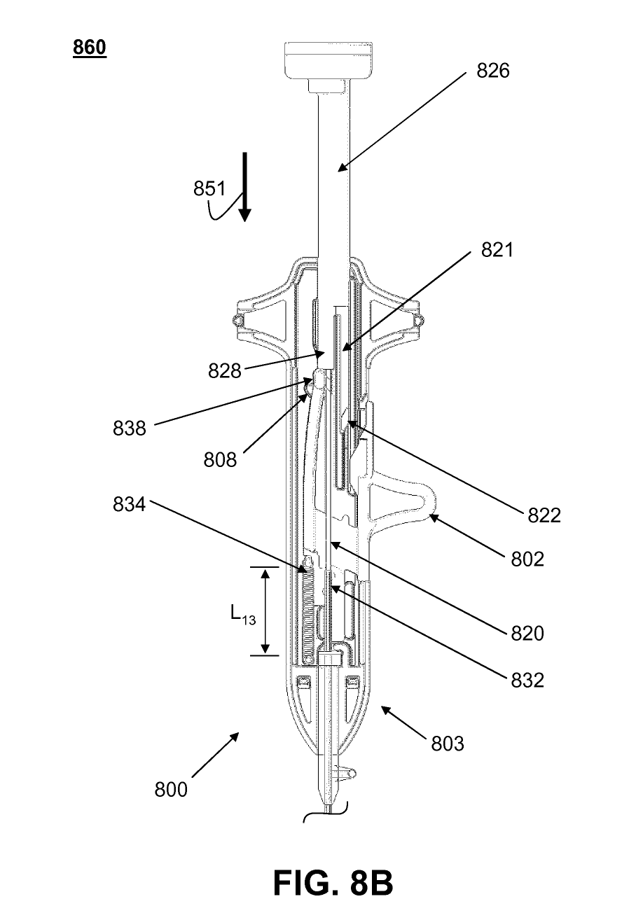

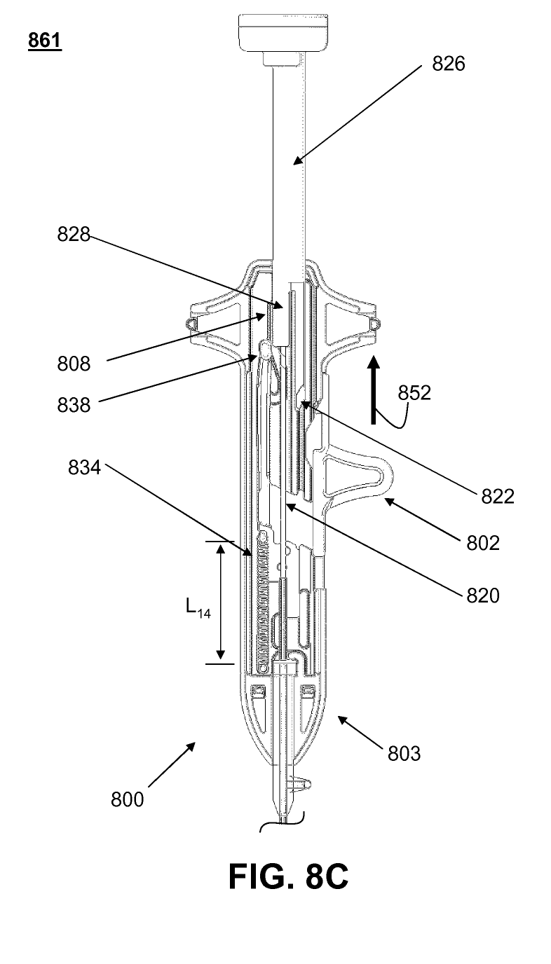

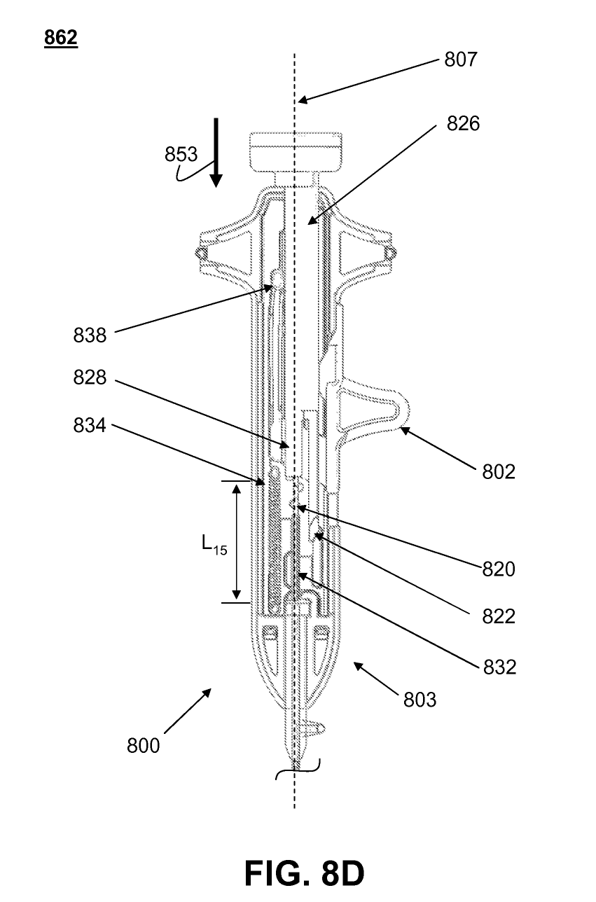

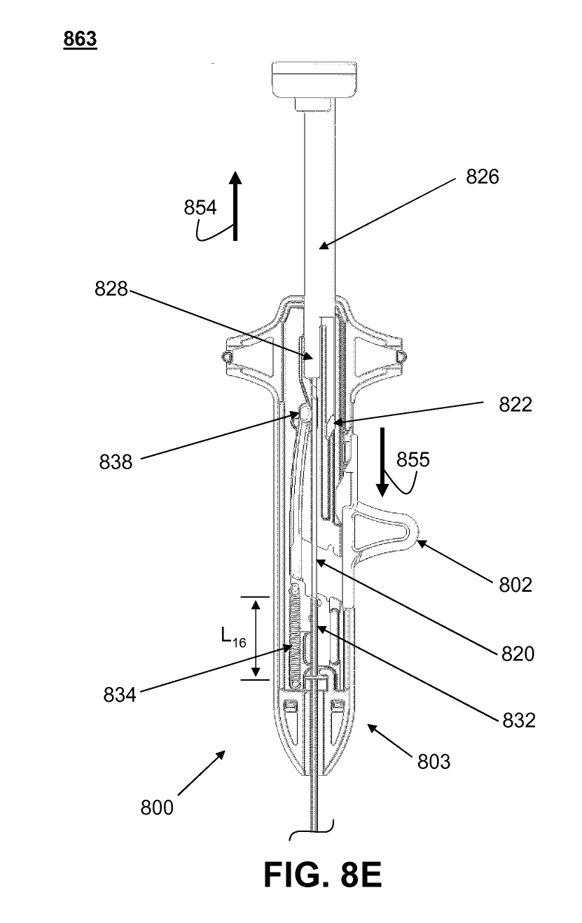

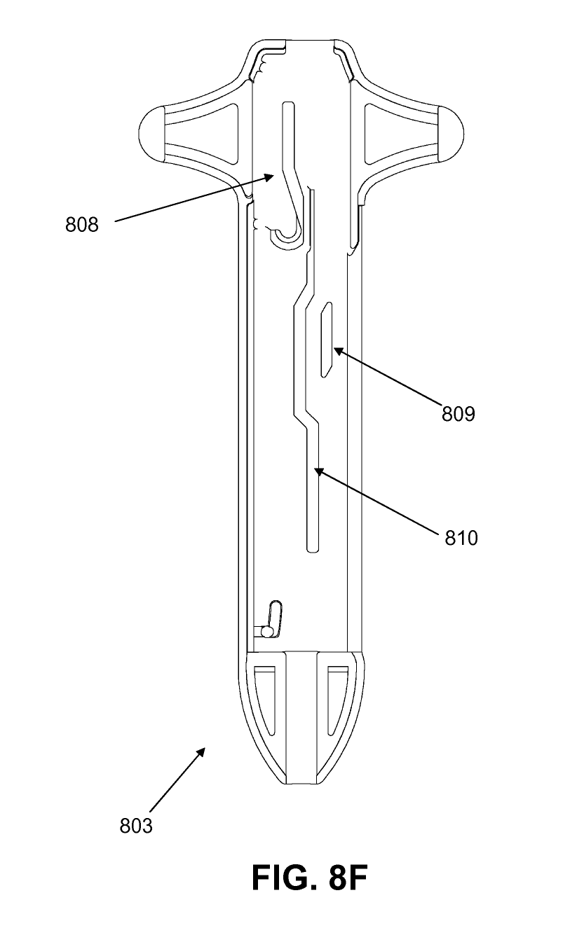

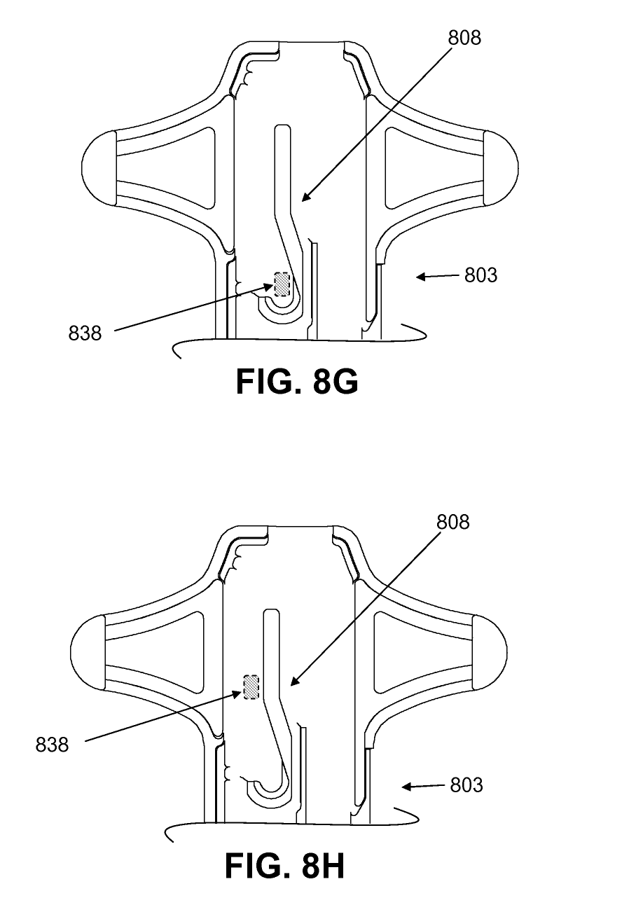

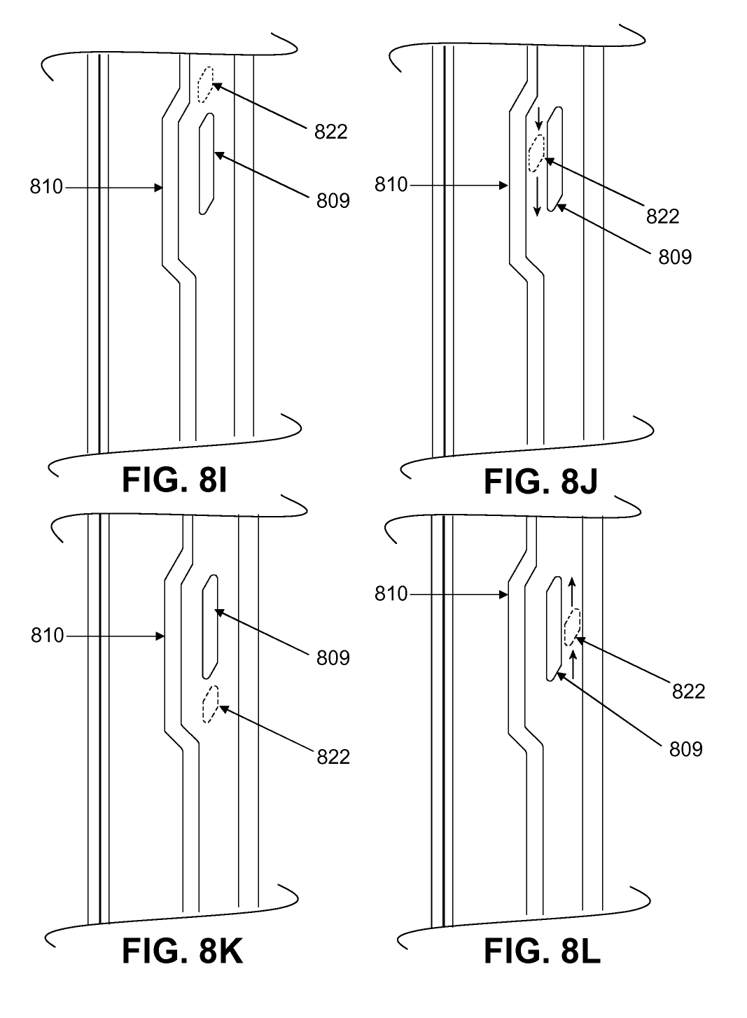

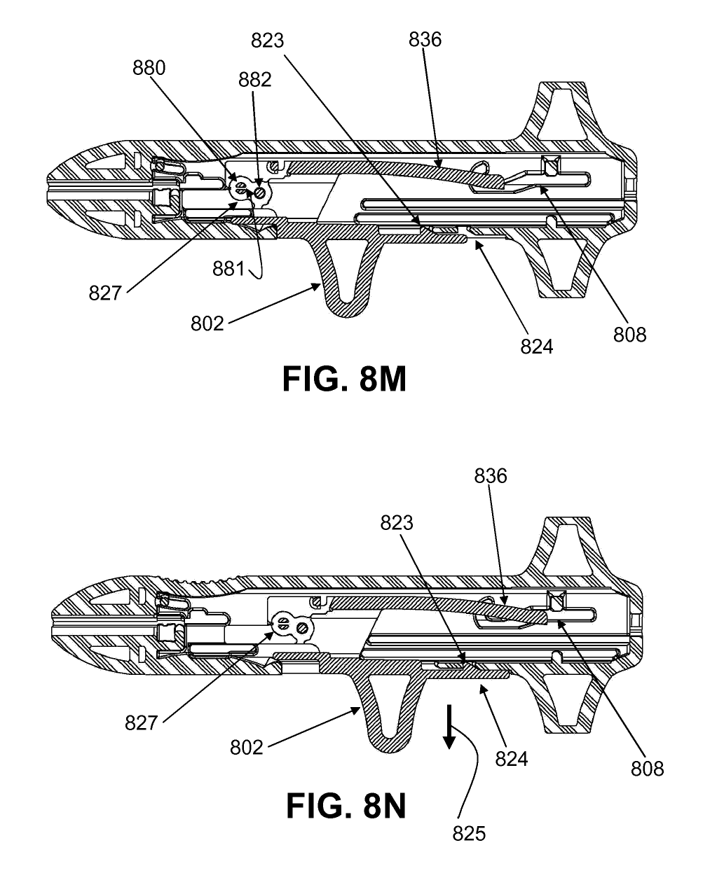

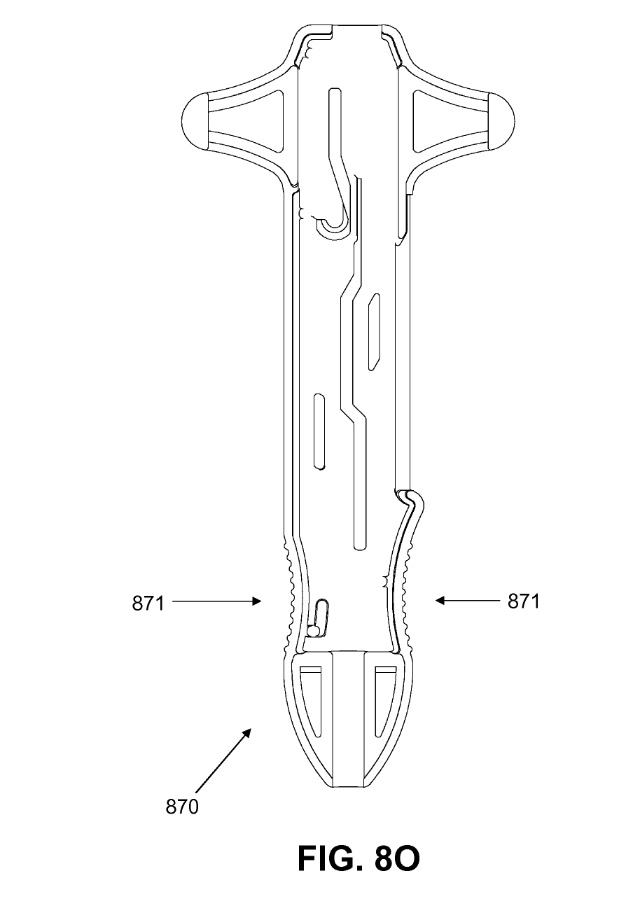

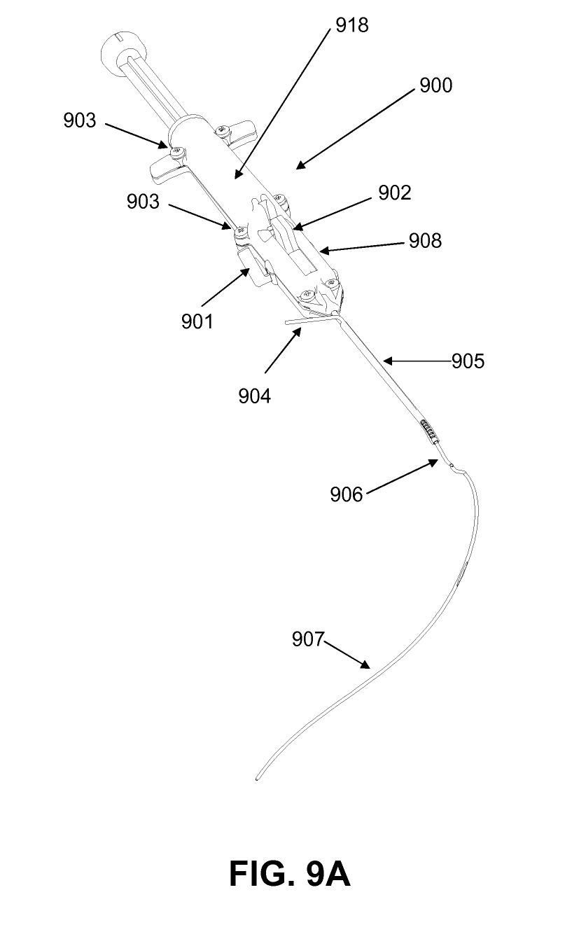

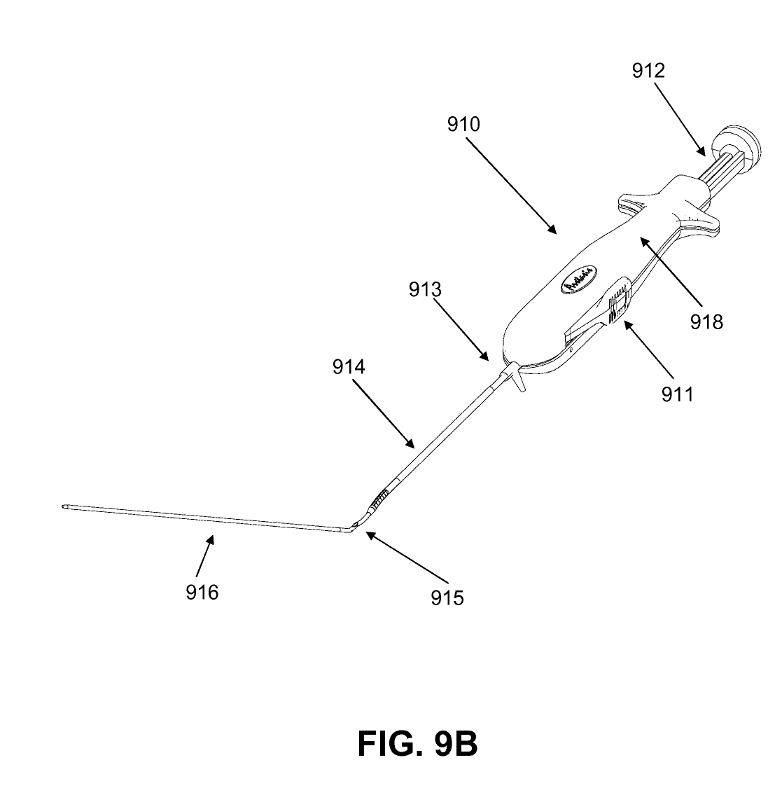

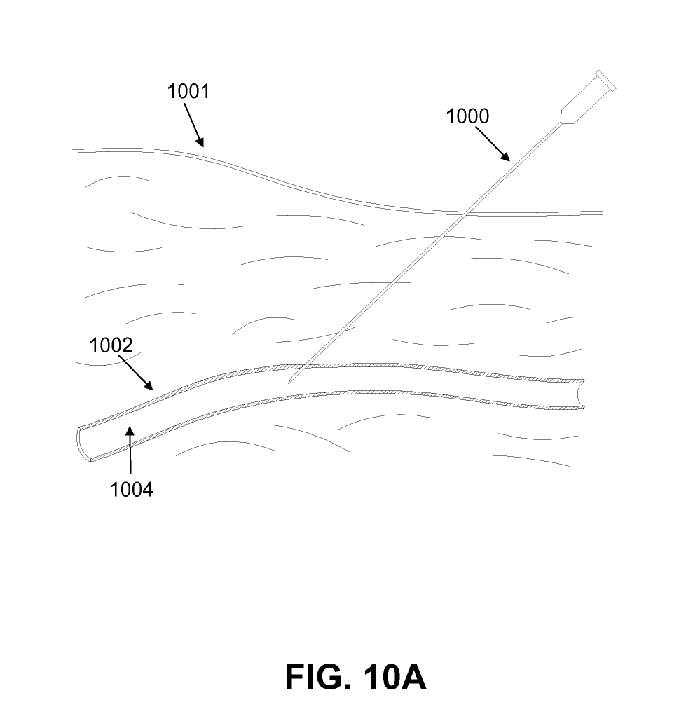

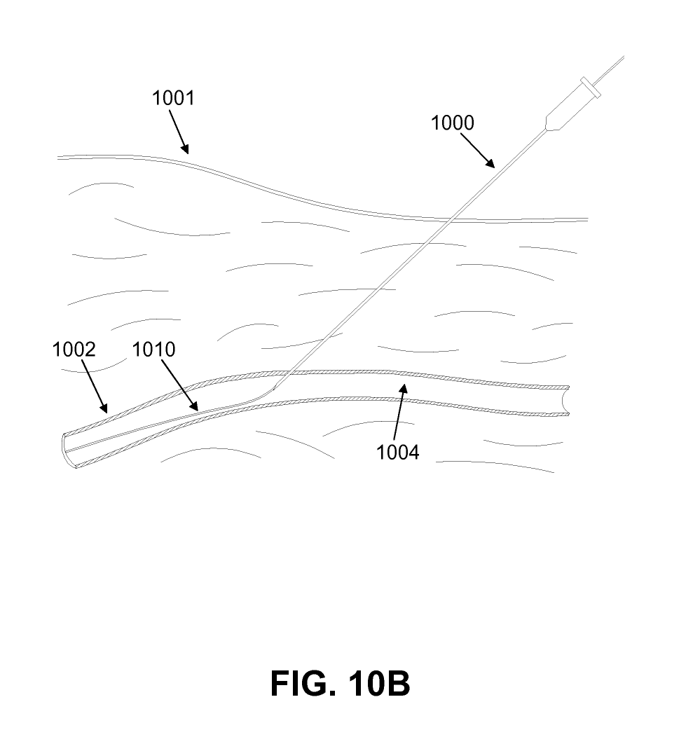

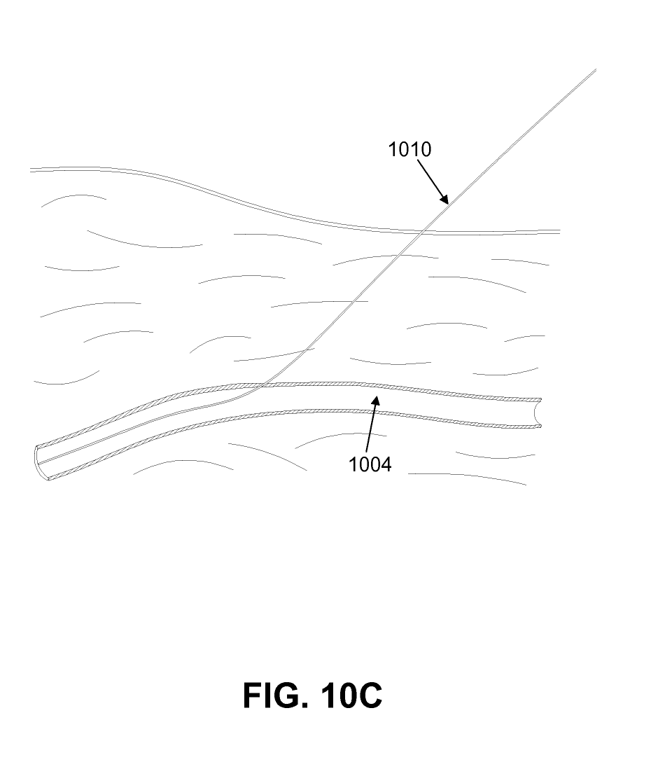

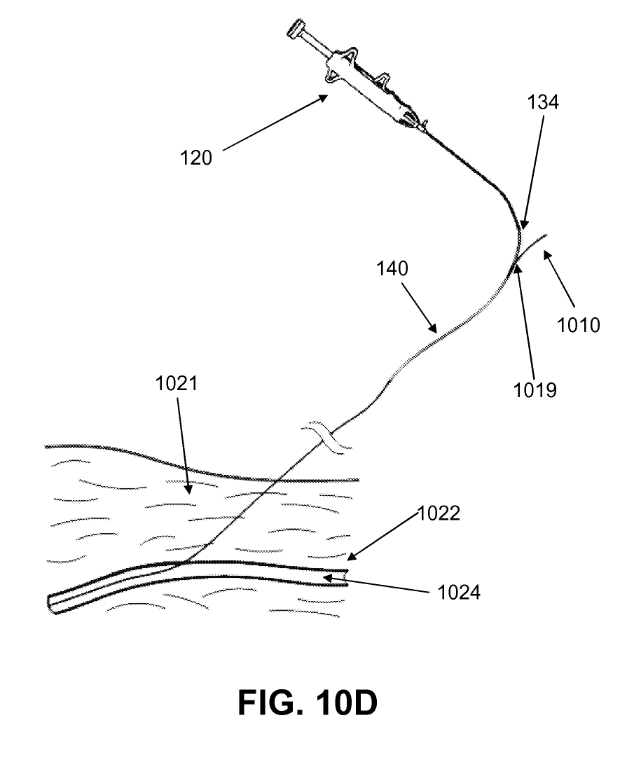

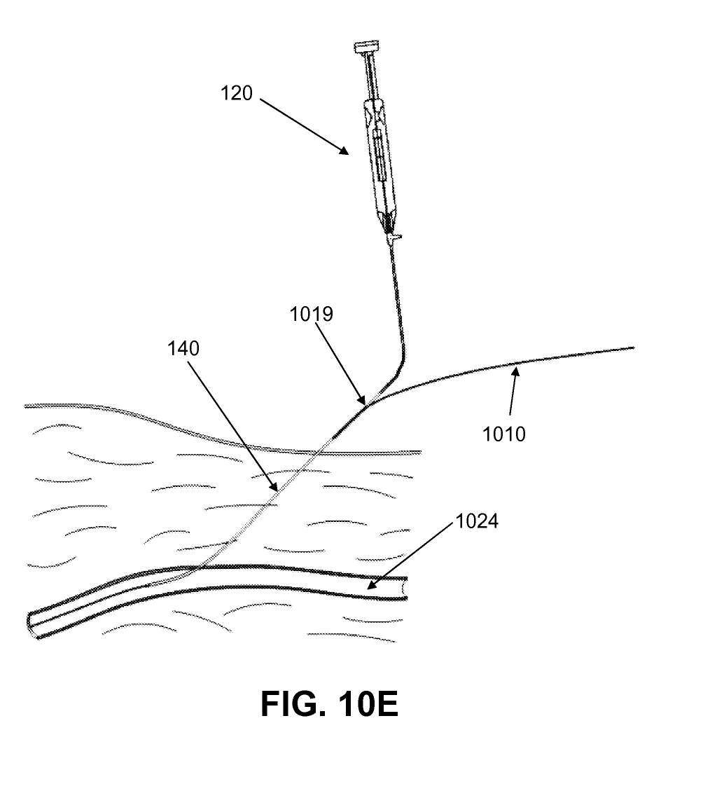

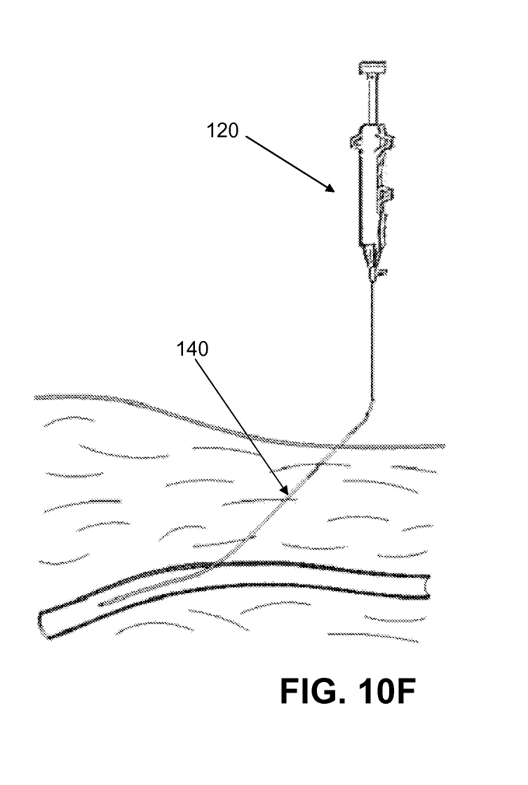

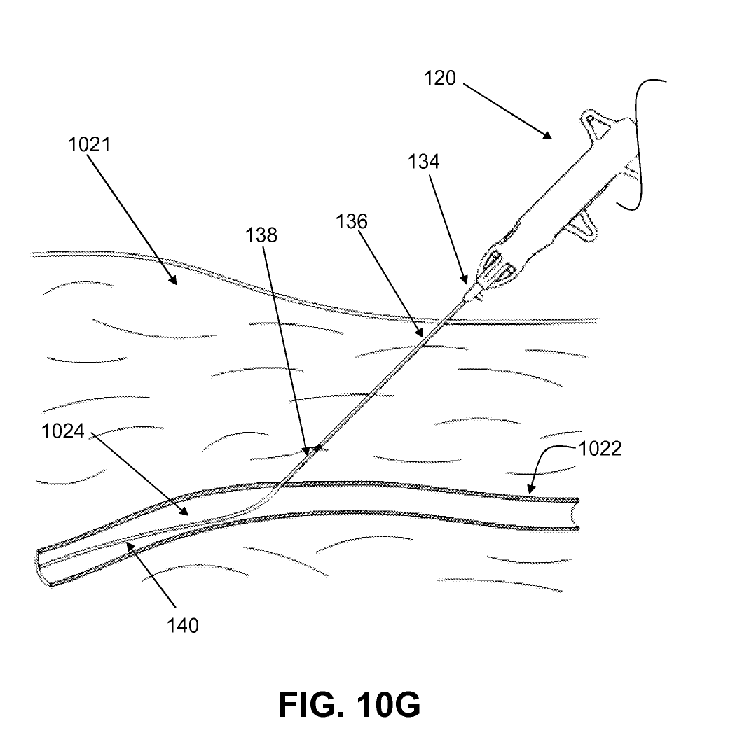

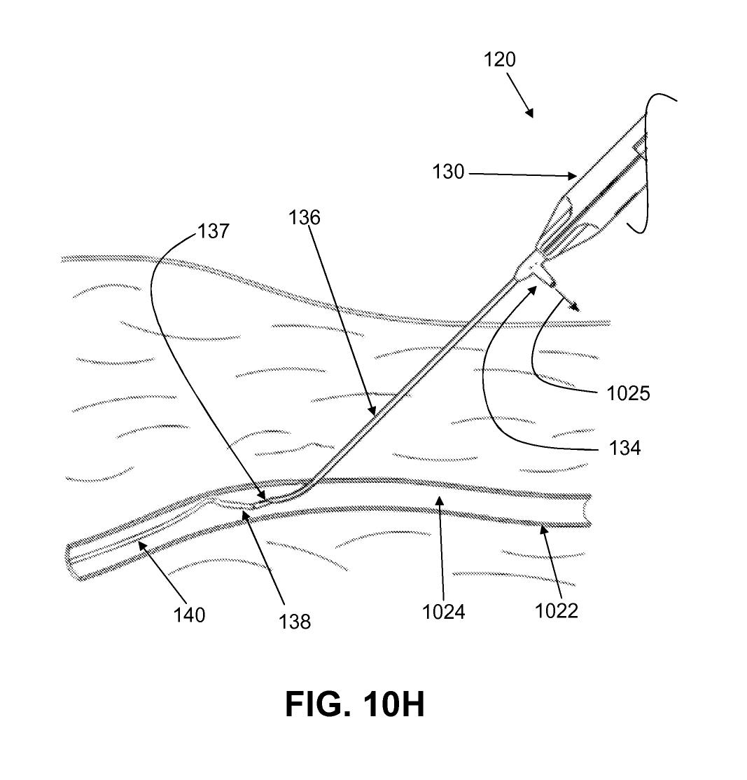

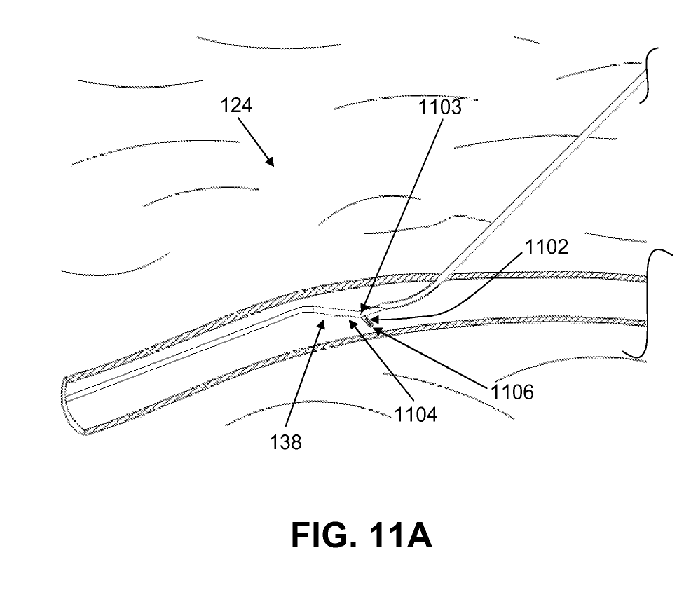

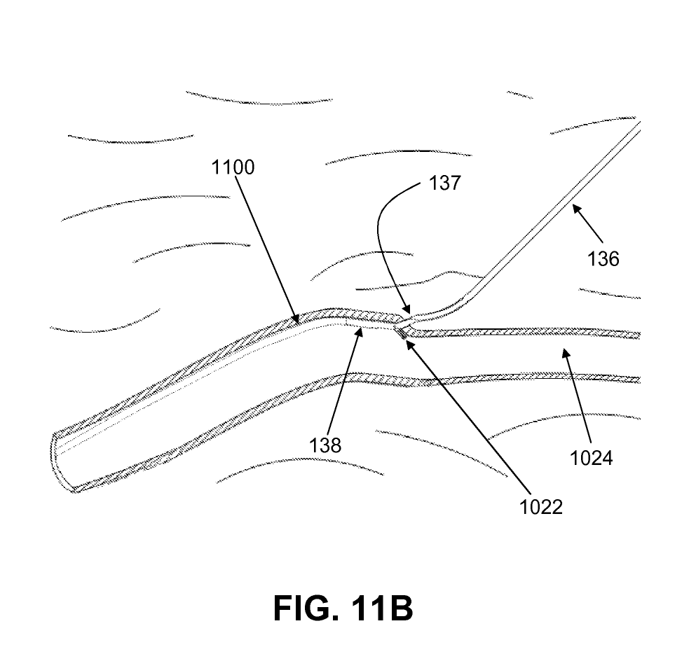

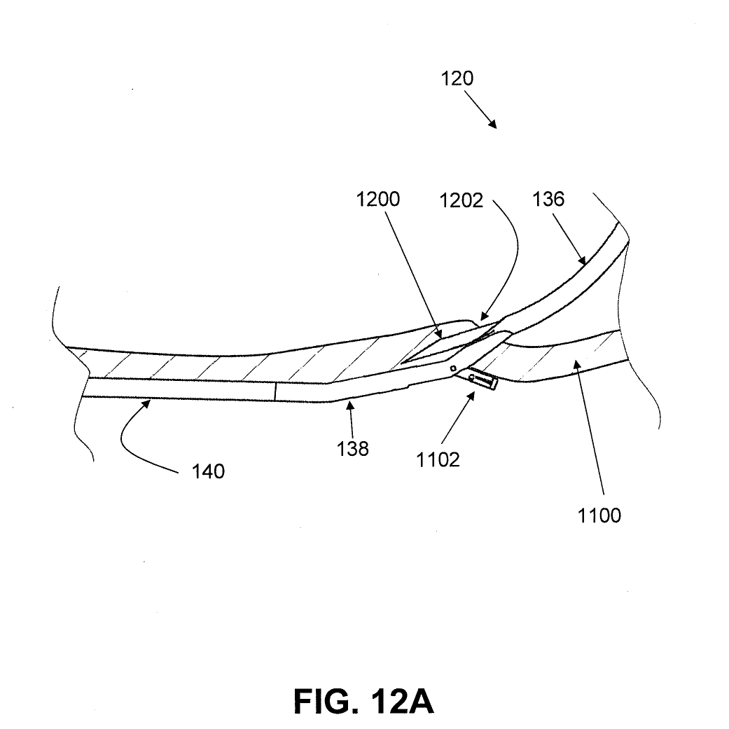

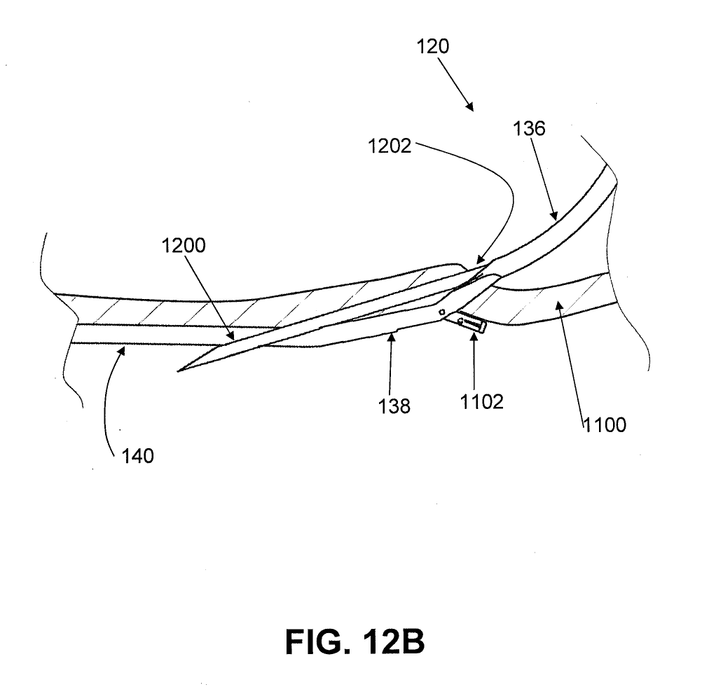

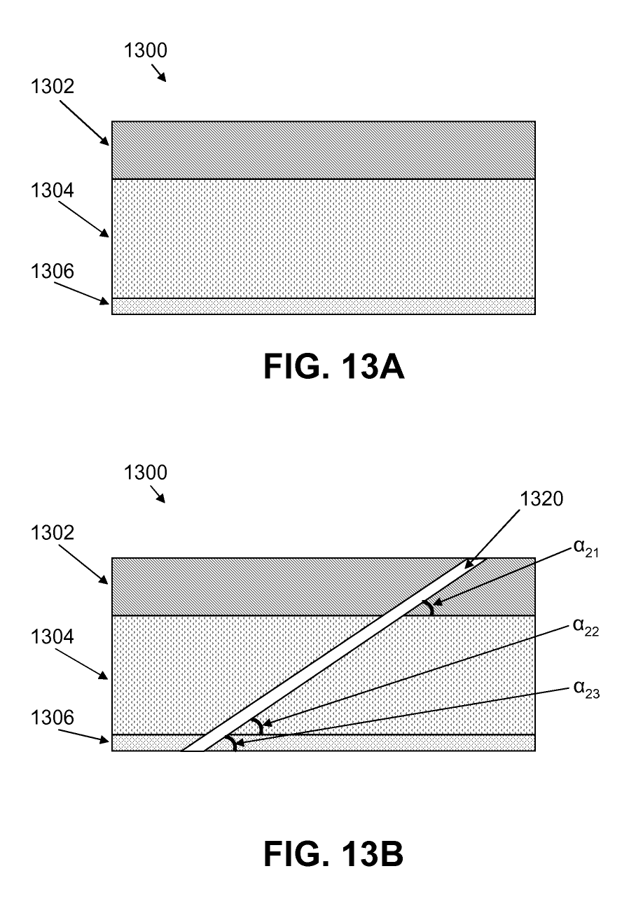

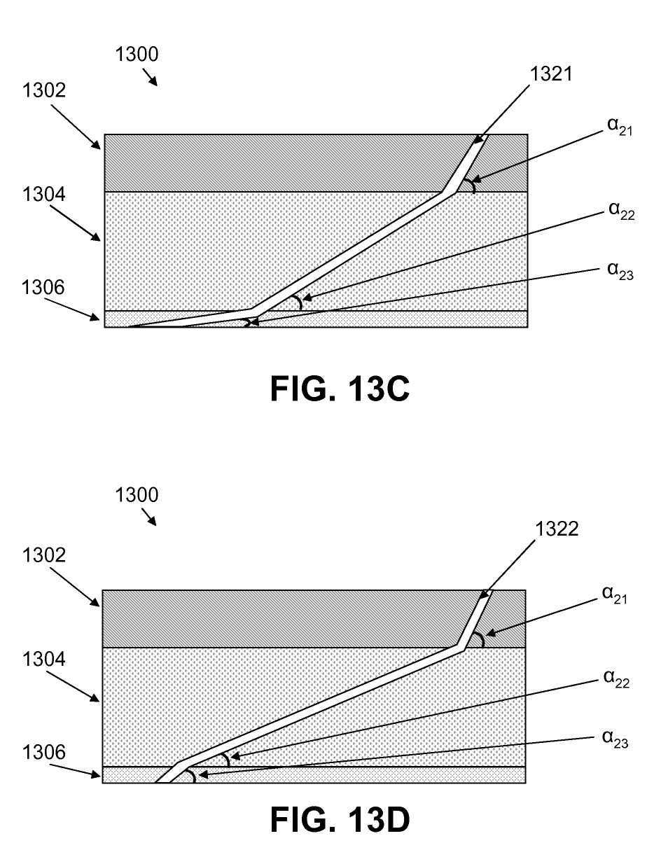

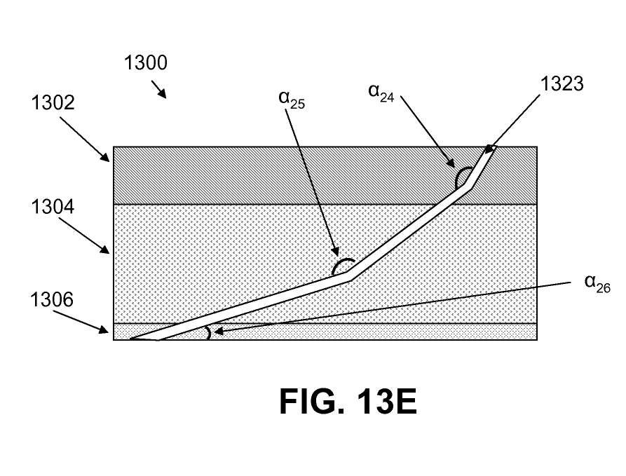

FIG. 61 shows another variation of a tissue tract-forming device (661). As shown there, tissue tract-forming device (661) comprises delivery guide (640) and a semi-tubular member (650) coupled to the external surface of delivery guide (640). Rather than having a substantially circular cross-section, semi-tubular member (650) has a somewhat U-shaped cross-section. Semi-tubular member (650) also has an actuating lumen (651) that houses an actuating cable (621). In some variations, semi-tubular member (650) may be stamped onto a shaft of delivery guide (640) during manufacturing. Using a semi-tubular member may, for example, help to maintain a relatively low overall profile for device (661).

While separately formed tubular or semi-tubular members and delivery guides have been described, in certain variations, a device may comprise an integrally formed tubular member and delivery guide.

A tissue tract-forming device may comprise one or more handles that may be used, for example, to actuate, control, position, and/or maneuver the device. Any appropriately configured handle may be used. As an example, FIG. 7A depicts a cutaway view of a portion of a tissue tract-forming device (709) comprising a delivery guide (700) and a handle (720) having a handle housing (708). Delivery guide (700) comprises a proximal portion having an aperture (702), and device (709) comprises a marker port (703) that encases a portion of delivery guide (700) in the location of aperture (702). Marker port (703), in turn, comprises an aperture (721) that is in fluid communication with aperture (702), and may be formed, for example, by a polymer overmolding process or any other suitable method. The size and shape of aperture (702) may be chosen, for example, to limit the likelihood that any polymer will enter the delivery guide during the overmolding process, in variations in which marker port (703) is formed by overmolding. In some variations, and as shown here, an overmolded marker port may include a stop portion (704) that limits movement of the marker port relative to the handle housing. As shown, the proximal portions of both marker port (703) and delivery guide (700) are secured within a handle housing (708). Stop portion (704) may help to secure and maintain the position of marker port (703) and delivery guide (700) within handle housing (708). Additionally, in some variations, delivery guide (700) may be fixedly coupled to stop portion (704), such that delivery guide (700) cannot rotate within stop portion (704). Furthermore, in certain variations, delivery guide (700) may be secured and positioned within handle housing (708) by other components and/or methods (e.g., mechanical junctions, form-fitting, screw-fitting, snap-fitting, adhesive-bonding, brazing, soldering, welding, heat-bonding, etc.).

Another variation of a marker port stop portion and delivery guide combination is shown in FIG. 7B. As shown there, a stop portion (714) and washer (715) together help secure the position of a delivery guide (700) and marker port (713). In some variations, washer (715) is attached to the delivery guide, and stop portion (714) is attached to the marker lumen. Washer (715) may be coupled to delivery guide (700) by, for example, being welded to the delivery guide. Alternatively or additionally, washer (715) may be soldered, form-fit, screw-fit, snap-fit, adhered, brazed, etc. to the delivery guide. This may help to hold the delivery guide in the handle (708). The stop portion (714) may help to align the marker lumen with the delivery guide. The size and shape of washer (715) may be varied, for example, to help securely position marker port (713) and delivery guide (700) within handle housing (708).

Referring again to FIG. 7A, delivery guide (700) comprises an opening (705) that allows a tissue-piercing member (706) to be inserted into a lumen of the delivery guide, such that the tissue-piercing member is slidable within the delivery guide. The length of tissue-piercing member (706) may be selected such that its distal end (not shown) extends from a tissue-piercing member port in a distal portion of the delivery guide. Tissue-piercing member (706) may be actuated (i.e., advanced within the delivery guide) using, for example, a pushing and/or pulling mechanism, such as a plunger.

As described previously, delivery guide may comprise a side aperture (e.g., aperture (702) in FIG. 7A) that provides access to a lumen of the delivery guide. In some variations, a marker port that is overmolded or otherwise formed over the delivery guide may comprise a channel or other opening that is aligned with the side aperture. One variation of such a marker port and side aperture combination is shown in FIG. 7C. As shown in the partial cut-away view of FIG. 7C, a marker port (713) is overmolded onto a delivery guide (700) that houses a tissue-piercing member (706). Marker port (713) comprises a projection (734) including a channel (732) terminating at an opening (736). Projection (734) may have any appropriate size and shape. For example, the projection may be tapered (as shown in FIG. 7C), and/or may have a standard shape that interfaces or fits with a syringe or tubing (e.g., the projection may be tapered to conform to the shape of a male or female Luer fitting). In some variations, projection (734) may alternatively or additionally include threads suitable for screwing in one or more additional components. As shown in FIG. 7C, projection (734) has a length L₁₂, which may be, for example, from about 6 millimeters to about 20 millimeters. As discussed above, aperture (702) of delivery guide (700) may be aligned with channel (732). This alignment may allow access from opening (736), through channel (732), and into the delivery guide via aperture (702).

Some variations of tissue tract-forming devices may comprise one or more tissue-piercing members having at least one lumen therethrough. The lumen may be used, for example, for the delivery of one or more therapeutic agents and/or other devices (e.g., a guidewire). For example, as shown in FIG. 7C, tissue-piercing member (706) comprises a side opening (744) and a plurality of side slots (742). Opening (744) may be used as an alignment feature during the manufacturing process, for example, to align tissue-piercing member (706) with the marker port during molding. Side aperture (702) of the delivery guide and channel (732) may be aligned with one or more of side slots (742) and/or side opening (744), thereby providing fluid communication from the tissue-piercing member lumen to the opening (736).

While tissue-piercing member (706) is depicted as having a certain number of side slots, a tissue-piercing member may have any appropriate number of side slots, such as 5, 10, 20, 30, 50, etc. side slots. In some variations, the number of side slots in a tissue-piercing member may be selected to allow access to the tissue-piercing member lumen across a length of the tissue-piercing member. In certain variations in which a tissue-piercing member is configured to slide within a delivery guide, the number of side slots along the length of the tissue-piercing member may correspond to the distance by which the tissue-piercing member may be translated. While slots have been depicted, in other variations, slits, mesh, and/or any fluid permeable material or configuration may alternatively or additionally be used. The plurality of side slots may provide guidance to a guide wire placed through the tissue-piercing member lumen. In some variations, a tissue-piercing member with side slots may be formed by molding, forging, and/or cutting the side slots from a hypotube needle. In certain variations, the number, size and/or shape of the side slots in a tissue-piercing member may be such that the slots do not interfere with the passage of fluids and/or devices in the tissue-piercing member lumen. Tissue-piercing member (706) may also have a single continuous side slot that allows fluid communication between the tissue-piercing member lumen and the marker port. The single side slot may be shaped (e.g., zig-zag shaped) to provide sufficient guidance to a guidewire placed through the tissue-piercing member lumen, while also preventing the guidewire from leaving the lumen.

As mentioned previously, tissue-piercing member (706) may be slidable within delivery guide (700). In one variation shown in FIG. 7D, the proximal portion of tissue-piercing member (706) is coupled to a pushing member (as shown, a plunger (750)) that actuates its movement. Tissue-piercing member (706) may be attached to plunger (750) by any of a number of appropriate methods, such as overmolding, mechanical junctions, form-fitting, screw-coupling, snap-fitting, adhesive-bonding, brazing, soldering, welding, heat-bonding, and the like. Additionally, a plunger may have any appropriate configuration. For example, as shown in FIG. 7D, plunger (750) comprises a grip (752), a plunger shaft (754), a first flange (756), a first flange tip (757), and a second flange (758). Grip (752) may be ergonomically sized and shaped. For example, grip (752) may be sized and shaped to readily accommodate a thumb, or to interface with an additional device, as will be described below. Some variations of plunger (750) may comprise at least one lumen (not shown) that extends from the attachment point of the tissue-piercing member through plunger shaft (754) to grip (752), such that the plunger lumen is in fluid communication with a lumen of tissue-piercing member (706). As depicted in FIG. 7D, tissue-piercing member (706) and plunger (750) may be at least partially retained in a handle housing (708). As also depicted in FIG. 7D, a marker port (703) may be overmolded onto delivery guide (700), and a stop portion (714) may be used to help secure marker port (703) and delivery guide (700), and may in turn be secured by a retaining structure (762). While the marker port may be overmolded onto the delivery guide, the marker port and delivery guide may be coupled using any suitable method that retains the delivery guide within the marker port.

As described above, some variations of plunger (750) may include at least one lumen. The lumen may, for example, extend from the attachment point of the tissue-piercing member, through plunger shaft (754), to grip (752). FIG. 7E shows plunger (750) including a lumen. More specifically, as shown there, plunger (750) comprises plunger shaft (754) and is at least partially retained within handle housing (708). Plunger shaft (754) comprises a lumen (not shown) terminating at an opening (776) at the proximal end (781) of the plunger shaft. During use, a tissue-piercing member (not shown) may be attached to plunger shaft (754), such that there is fluid communication between a lumen of the tissue-piercing member and the lumen in the plunger shaft.

In some variations, opening (776) may be sized and shaped to accommodate the opening of a syringe, such as opening (782) of syringe (780). For example, syringe opening (782) may be a mechanical counterpart to plunger opening (776), such that the two openings can mechanically couple to each other (e.g., via a Luer-lok™, Luer-slip™, lock-fit, snap-fit, or friction-fit). When syringe (780) is coupled to plunger (750), lumens of the tissue-piercing member and plunger (750), as well as the barrel of syringe (780), may be in fluid communication with each other as a result. Syringe barrel (784) may, for example, contain any suitable material (e.g., a fluid or gas composition) suitable for introduction through plunger (750), into a tissue-piercing member lumen, into a delivery guide, and into tissue. For example, in some variations, syringe barrel (784) may contain a saline flush solution, one or more therapeutic agents, one or more gases (e.g., oxygen, carbon dioxide, nitrogen), one or more contrast agents, or the like. The rate at which the agent(s) may be introduced to the tissue may be manually regulated, or regulated by a computer or other mechanism. Alternatively or additionally, opening (776) may be used to introduce one or more devices into a target tissue or newly formed tissue tract. For example, one or more catheter-based devices may be delivered through opening (776), through a tissue-piercing member lumen, and into tissue. In some variations, a guide wire may be inserted through opening (776). While opening (776) is depicted in FIG. 7E as having a round shape, such an opening may have any appropriate shape, such as a tapered shape that may be fitted with a Luer-type fitting. In some variations, opening (776) and ring-structure (778) may be configured to form a mechanical lock with a device having a complementary shape.

As described above, in certain variations, a tissue-piercing member and plunger assembly may be at least partially contained within a handle housing. In some variations, additional components in the housing may regulate the actuation of the tissue-piercing member and/or plunger. One variation of a handle housing and handle components is shown in FIG. 8A, which provides a cut-away view of a proximal portion of a tissue tract-forming device (800). As shown there, handle housing (803) comprises a lever aperture (801), brackets (804), an attachment protrusion (833), and a retaining structure (806). In some variations, handle housing (803) may be in the form of a single molded shell, while in other variations, handle housing (803) may comprise two or more molded shells that are coupled together. Brackets (804) comprise protrusions (805), which may be used, for example, to couple multiple components of handle housing (803) together (e.g., by a snap-fit or friction-fit). Alternatively or additionally, features (such as threaded apertures, grooves, protrusions, hooks, etc.) may be provided in the handle housing so that the multiple components may be coupled together by mechanical junctions, form-fitting, screw-fitting, snap-fitting, adhesive-bonding, brazing, soldering, welding, heat-bonding, and the like.

Examples of materials which may be suitable for use in handle housing (803) include polymers, such as polyacetals (e.g., DELRIN® acetal resin), polystyrene, polyetheretherketone (PEEK), polyetherketoneketone (PEKK), polyethylene, acrylonitrile butadiene styrene (ABS), polyethylene terephthalate (PET), polycarbonates, polytetrafluoroethylene (e.g., TEFLON® polymer), polyimides, nylons, silicone, SANTOPRENE® thermoplastic vulcanizates, and polyvinyl chloride (PVC). Some types or families of polymers may be available in different durometers or hardnesses, and in such cases the appropriate polymer or polymers for the desired characteristics may be used. Examples of materials which may be relatively rigid include PEEK, PEKK, ABS, or silicone, and examples of materials which may be relatively soft include silicone, SANTOPRENE® thermoplastic vulcanizates, and PEBAX® polymers. Of course, these are only exemplary materials, and other relatively rigid or relatively soft materials may also be used, as appropriate.

Additionally, materials that are not especially soft or rigid may be used. Moreover, in some variations, combinations (e.g., mixtures) of different materials may be used. For example, a blend of polymers may be used, or a composite of one or more polymers and filler materials (e.g., glass fibers and/or particles, carbon fibers, etc.) may be used. Lubricants, for example, silicone oils and/or PTFE, may be added to various components (e.g., the plunger and/or any levers or actuators) to reduce any frictional interactions between moving parts.

Referring again to FIG. 8A, device (800) includes a lever (802) that is partially retained within handle housing (803) and that protrudes out of lever aperture (801) in the handle housing. Device (800) also includes a marker port (830), a delivery guide (832), a tissue-piercing member (820), and a plunger (826), all partially retained by handle housing (803). Plunger (826) comprises a first flange (821), a first flange tip (822), and a second flange (828). In some variations, first flange (821) may be longer than second flange (828). As depicted in FIG. 8A, first flange tip (822) is shaped as a parallelogram. However, a first flange tip may have any suitable shape. Lever (802) comprises a notch (835), a stop-arm (836), a stop-arm base (837), and a stop-arm head (838). A retainer cable (not shown here, but see FIGS. 5B and 6D-6I) may be attached to a portion of stop-arm base (837), and/or a portion of stop-arm head (838). During use, lever (802) may be actuated to translate stop-arm (836), stop-arm base (837), and stop-arm head (838), and may also be used to actuate an attached retainer cable in a similar way.

Device (800) may further comprise a spring (834) disposed within handle housing (803) and coupled to attachment protrusion (833) and notch (835) of lever (802). In some variations, spring (834) may have a spring constant that biases lever (802) into the position shown.

Handle housing (803) and the components as described may be used to regulate the movement of plunger (826) and tissue-piercing member (820) within delivery guide (832) during use of device (800). Of course, other appropriate variations of handles and actuation mechanisms may also be used.

FIGS. 8B-8E depict different configurations and arrangements of the components of device (800) retained by handle housing (803), during the actuation of plunger (826) and tissue-piercing member (820).

First, FIG. 8B shows a configuration (860), in which the position of lever (802) blocks any movement of plunger (826) or tissue-piercing member (820) in the direction of arrow (851). When stop-arm head (838) is in a plunger-obstructing position, as in configuration (860), plunger (826) is prevented from being advanced in the direction of arrow (851). Stop-arm head (838) obstructs the movement of plunger (826) by contacting second flange (828) of plunger (826) and a curved ramp (808) in the handle. Spring (834) may be biased such that lever (802) is retained in the position depicted in FIG. 8B, and may have a length L₁₃, which may be, for example, from about 6 millimeters to about 20 millimeters. For example, the above-described lengths may be partially determined by the size (e.g., thickness, length, etc.) of the tissue in which the tract is to be formed. When device (800) is in configuration (860), tissue-piercing member (820) is prevented from being actuated into tissue. Configuration (860) may be, for example, an initial configuration of the overall device, prior to the formation of a tract in tissue.

FIG. 8C depicts another configuration (861) of the components in handle housing (803). In some variations, configuration (861) may be obtained from configuration (860) by advancing lever (802) in the direction of arrow (852). Advancing lever (802) in the direction of arrow (852) moves stop-arm head (838) in the same direction, and may cause spring (834) to expand to a length L₁₄ which may be, for example, from about 11 millimeters to about 25 millimeters. Advancing the lever in this way may cause it to catch on a protrusion on the handle which may maintain its position, which will be described in detail later on. Typically, length L₁₄ may be greater than length L₁₃ of configuration (860). When stop-arm head (838) is moved in the direction of arrow (852), a curved ramp (808) deviates the stop-arm head away from second flange (828) of plunger (826). Curved ramp (808) has a shape that may retain stop-arm head (838) in both a first plunger-obstructing position and a second plunger-passing position. For example, curved ramp (808) may be molded into handle housing (803) in the shape of an angled question mark, which will be described in detail later. When stop-arm head (838) is positioned along one side of second flange (828) as shown in FIG. 8C, it is in a plunger-passing position, since the path of plunger (826) is unobstructed, thereby allowing plunger (826) to be advanced. Advancing lever (802) in the direction of arrow (852) may also actuate a retainer. For example (and referring to FIG. 5A), moving lever (802) as described may tension cable (507), which, in turn, may pivot retainer (500) out of slot (511). This configuration may position the retainer to engage and/or secure tissue for tract formation.

FIG. 8D depicts another configuration (862) of the components in handle housing (803), which may be obtained from configuration (861) by, for example, advancing plunger (826) in the direction of arrow (853). Advancing plunger (826) in the direction of arrow (853) advances tissue-piercing member (820) in the same direction, into delivery guide (832). Advancing plunger (826) also causes first flange tip (822) to move toward and along the midline (807) of handle housing (803) (i.e., away from lever (802)) as the first flange tip is advanced in the direction of arrow (853). In some variations of configuration (862), tissue-piercing member (820) may be in a tissue-penetrating position. Additionally, spring (834) may have a length L₁₅, which may be, for example, from about 11 millimeters to about 25 millimeters. In certain variations, length L₁₅ may be equal to length L₁₄ (FIG. 8C).

FIG. 8E depicts a configuration (863) of the components in handle housing (803), which may be obtained from configuration (862) by retracting plunger (826) in the direction of arrow (854). Retracting plunger (826) in the direction of arrow (854) may cause lever (802) to move in the direction of arrow (855), thereby drawing stop-arm head (838) into the plunger obstructing position (similar to the position shown in FIG. 8B). Retracting plunger (826) may also cause first flange tip (822) to move toward lever (802) as the first flange tip is retracted in the direction of arrow (854). As first flange tip (822) is retracted, it may contact and move a portion of lever (802), such that lever (802) may be released in the direction of arrow (855). Here, stop-arm head (838) is in the path of second flange (828), which prevents the second flange from moving past stop-arm head (828) in the direction of arrow (855). Alternatively or additionally, stop-arm head (838) may be drawn into the plunger obstructing position by spring (834). In configuration (863), spring (834) has a length L₁₆, which may be, for example, from about 6 millimeters to about 26 millimeters. In certain variations, length L₁₆ may be equal to length L₁₃. Once lever (802) has moved into the plunger-obstructing position, plunger (826) and tissue-piercing member (820) may be prevented from being actuated in the direction of arrow (855). In some variations, as lever (802) is moved in the direction of arrow (855), the tension on a retainer cable (e.g., cable (507) of FIGS. 5A and 5B) may be released, allowing retainer (500) to pivot into slot (511). With the retainer seated in the slot, the device may be withdrawn from the tissue. It should be understood, however, that devices described here may also be withdrawn when in other configurations.

A handle housing such as handle housing (803) may have any configuration suitable for accommodating various device components. For example, FIG. 8F shows an interior surface of a portion of the handle housing (803) depicted in FIGS. 8B-8E. Handle housing (803) may include multiple protrusions and ramps that may, for example, aid in the various configurational changes the device may assume during use (e.g., as described above). It should be understood that while handle housing (803) has a certain arrangement of protrusions and ramps, other variations of handle housings may have different arrangements and/or may retain different types and/or numbers of components, as appropriate. The protrusions and ramps in the handle housing may be formed by molding, carving, or any appropriate method. For example, FIG. 80 illustrates another variation of a handle housing (870) that comprises a different arrangement of molded protrusions and ramps and that may also be used in a tissue tract-forming device. As shown there, handle housing (870) also comprises protrusions (871), which may act as finger grips to help ensure that the device is gripped at a prescribed location.

A mirror-image of handle housing (803) is depicted in FIG. 8F. As shown there, handle housing (803) comprises a curved ramp (808), a protrusion (809), and a rail (810). These features may be integrally formed with handle housing (803), for example, molded, or may be separately formed and affixed to handle housing (803) using any suitable method (e.g., mechanical junctions, form-fitting, screw-fitting, snap-fitting, adhesive-bonding, brazing, soldering, welding, heat-bonding, and the like). Curved ramp (808) may be curved in at least a portion of the ramp, and substantially straight in another portion of the ramp. Rail (810) and protrusion (809) may be arranged such that an object may pass therebetween. For example, rail (810) and protrusion (809) may be positioned generally parallel to each other, as shown in FIG. 8F. Protrusion (809) may have any shape or form that is configured to direct the motion of an object in two different directions, with each direction corresponding to an edge of the protrusion. For example, protrusion (809) is depicted as a parallelogram, which may first direct an object at an angle with respect to rail (810), and then direct the object parallel to rail (810). In some variations, during operation of a tissue-tract forming device comprising handle housing (803), curved ramp (808) may guide the position of a lever stop-arm head of the device, and rail (810) and protrusion (809) may guide the position of a plunger first flange tip of the device. Alternatively or additionally, one or both of these components may guide the position of one or more other components of the device.

FIGS. 8G-8L illustrate the relative positions of certain components of device (800) during use.

First, FIG. 8G depicts the position of a lever stop-arm head (838) of the device within housing (803) when the lever stop-arm head is in a plunger-obstructing position. The lever stop-arm head may be in this position, for example, when the device is in configuration (860) or (863) shown above. In this configuration, actuation of a pushing member (e.g., a plunger) and a tissue-piercing member (e.g., a needle) of the device may be prevented. In FIG. 8G, stop-arm head (838) is seated firmly in the curved portion of curved ramp (808), which may obstruct the path of a second flange of the pushing member (see, e.g., FIG. 8B). FIG. 8H shows the position of stop-arm head (838) when it is in the plunger-passing position. The stop-arm head (838) may be in this position, for example, when the device is in configuration (861) or (862), which may allow the unobstructed longitudinal movement of the plunger and tissue-piercing member into and out of the delivery guide (see FIGS. 8C and 8D). Stop-arm head (838) may be urged into the plunger-passing position when the lever is actuated longitudinally, for example, according to arrow (852) as indicated in FIG. 8C. The lever stop-arm head may allow the lever to “lock” the plunger into position (e.g., as a safety feature to prevent premature tissue-piercing member actuation).

FIGS. 8I-8L depict different positions of a plunger component within housing (803) of device (800) when device (800) is in use. As shown there, the direction of movement of the plunger and tissue-piercing member are guided by rail (810) and protrusion (809) via plunger first flange tip (822). FIG. 81 depicts the position of first flange tip (822) when the plunger is fully retracted (see, e.g., configurations (860) and (863) in FIGS. 8B and 8E). FIG. 8J depicts the position of first flange tip (822) as the plunger is being advanced (see, e.g., arrow (853) in FIG. 8D). As the plunger is advanced, first flange tip (822) may be urged between rail (810) and protrusion (809), because of the sliding edges of the first flange tip (822) parallelogram and protrusion parallelogram. FIG. 8K shows the position of first flange tip (822) after the plunger has been fully advanced (see, e.g., configuration (862) in FIG. 8D). Finally, FIG. 8L shows the position of first flange tip (822) as the plunger is being retracted (see, e.g., arrow (854) in FIG. 8E). As the plunger is retracted, first flange tip (822) may be urged between protrusion (809) and the edge of housing (803), due to the sliding edges of the first flange tip (822) parallelogram and the protrusion parallelogram. As the first flange tip (822) is retracted, it may interact with a portion of the lever releasing its engagement with the handle housing and allowing the spring to urge it in the direction of arrow (855) (see, e.g., lever (802), which actuates stop-arm head (838)), as shown in FIG. 8E.

FIGS. 8M and 8N depict one mechanism by which the lever may be disengaged with the handle housing so that the spring may urge it in the direction of arrow (855), which is shown in FIG. 8E. FIG. 8M depicts the position of lever (802), lever protrusion (823), and stop arm (836) with respect to housing groove (824) and curved ramp (808) in a first configuration (e.g., configuration (860) of FIG. 8B). Lever protrusion (823) may be any desired shape (e.g., triangular). Housing groove (824) is sized and shaped to receive and retain lever protrusion (823). When lever (802) is urged in the direction of arrow (852) as shown in FIG. 8C, lever protrusion (823) is also urged in the direction of arrow (852) into housing groove (824). FIG. 8N depicts the position of the lever protrusion as it is retained in housing groove (824), which is also illustrated in FIGS. 8C and 8D. When the first flange tip is drawn in the direction of arrow (854) in FIG. 8E (also illustrated in FIG. 8L), the first flange tip contacts handle (802), which urges the lever protrusion (823) in the direction of arrow (825) shown in FIG. 8N. Once lever protrusion (823) has been deflected in the direction of arrow (825), it is no longer retained in housing groove (824), and the spring (834) may urge the lever back into the position shown in FIG. 8M. While an exemplary mechanism used to engage and disengage the lever with the handle housing is described here, other mechanisms may also be used as appropriate.

Also shown in FIGS. 8M and 8N are cable attachment features (827), which are coupled to, or integral with, lever (802). Cable (507) from the retainer shown in FIGS. 5A and 5B may be threaded through the device to cable attachment features (827), which comprise a first post (880) and a second post (881). The cable may be threaded through the device as shown, for example, in FIGS. 6D-6I. Once in the handle housing, cable (507) may be wound around cable attachment features (827). For example, cable (507) may be run through a slit (881) of first post (880). The cable may be heat-staked (e.g., melting the first post to close the slit over the cable) to first post (880) under tension. Additional attachment may be provided by wrapping and heat-staking the cable around second post (882). Securing the cable to first and second posts may help the cable to remain secured to lever (802) (e.g., to withstand forces of about one pound). Other ways to secure the cable to lever (802) may include, for example, coupling the cable to the cable attachment feature (e.g., via brazing, soldering, welding, heat-bonding, etc.).

While one variation of a device for forming tracts in tissue has been described, other variations of devices for forming tracts in tissue may be used, as shown, for example, in FIGS. 9A and 9B. FIG. 9A depicts a device (900) comprising levers (901) and (902), a marker port (904), a tissue-piercing member such as a needle (not shown), a delivery guide (905), an anchor member (906), and a guide sheath (907). Device (900) comprises a housing (908) formed by at least two components coupled to each other by a plurality of screws (903). However, in some variations, housing (918) may be assembled using adhesive bonding, snap-fitting, friction-fitting, or the like, or may be integrally formed. FIG. 9B depicts another variation of a device (910), which comprises a housing (918), a slide actuator (911), a plunger (912), a marker port (913), a tissue-piercing member such as a needle (not shown), a delivery guide (914), an anchor member (915), and a guide sheath (916). In some variations, housing (918) (or any other housing, as appropriate) may be formed in such a way that screws are not required for assembly.

As discussed above, devices described here may be used to form one or more tracts in tissue. FIGS. 10A-10H depict one variation of a method and device used to access tissue in order to form one or more tracts in the tissue. The method shown in FIGS. 10A-10H may be used in conjunction with other methods (e.g., methods shown in FIGS. 11A and 11B and FIG. 12) to form one or more tracts in tissue. While these figures show the formation of a tract in arterial tissue, it should be understood that the devices and methods described here may be used with any suitable tissue, as described above.

FIGS. 10A-10C show a standard Seldinger procedure for placement of a wire through a tissue. First, and as shown in FIG. 10A, a needle (1000) is advanced through subcutaneous tissue (1001) into an artery (1002). As shown, needle (1000) has entered a lumen (1004) of artery (1002). Entry into lumen (1004) by needle (1000) may optionally be visually confirmed by observing a flash of blood (i.e., blood flow) through the needle, for example, through a port in the needle (e.g., a marker port). FIG. 10B shows advancement of a wire (1010) through needle (1000) and into lumen (1004) of artery (1002). After placement of wire (1010), the needle may be withdrawn proximally, leaving wire (1010) in lumen (1004), as shown in FIG. 10C. Devices, such as the ones described above, may access the lumen via wire (1010). Optionally, such devices may also be used to perform a standard Seldinger procedure to gain initial access to the lumen, so that a tract may be formed through tissue.

FIGS. 10D-10H show how the device (120) of FIG. 1 may be used to access a tissue lumen. As shown in FIG. 10D, device (120) is inserted through subcutaneous tissue (1021) and into a lumen (1024) of an artery (1022). Of course, while the methods described here are shown with specific reference to an artery, it should be understood that, as described above, the methods may be used with any suitable tissue. A wire (1010) (previously positioned using a standard Seldinger procedure as described above with reference to FIGS. 10A-10C) may be threaded through guide sheath (140), exiting via a side port (1019). FIG. 10E shows the advancement of guide sheath (140) over wire (1010) as a guide for placement into lumen (1024). After guide sheath (140) has been placed in lumen (1024), wire (1010) may be removed, as shown in FIG. 10F.

As will be described in more detail below, in some variations, device (120) may also be rotated during insertion. For example, device (120) as shown in FIG. 10F has been rotated about 45° from the position shown in FIG. 10E, and device (120) shown in FIG. 10G has been rotated an additional 45° (for a total rotation of about 90° from the position shown in FIG. 10E). Of course, as will be described below, any degree of rotation, in any direction, may be used as desirable, and in some cases, it may be preferable not to rotate device (120) at all.

FIG. 10G illustrates further advancement of device (120) into tissue (1021). As shown there, device (120) has been advanced so that delivery guide (136) and anchor member (138) have entered subcutaneous tissue (1021) and anchor member (138) is beginning to enter lumen (1024) of artery (1022). Additionally, the distal portion of guide sheath (140) has been advanced into lumen (1024). Once anchor member (138) enters lumen (1024), tissue-piercing member port (137) may become exposed to blood flowing through lumen (1024). As described previously, tissue-piercing member port is in fluid communication with the marker port (134), so that blood entering the tissue-piercing member port may exit through marker port (134), thereby indicating that anchor member (138) has been correctly positioned in lumen (1024) (by a flash of blood (1025)). FIG. 10H shows how device (120) has been rotated back 90°, to the original advancement position shown in FIG. 10E. As also shown in FIG. 10H, anchor member (138) has been advanced so that it fully resides within lumen (1024) of artery (1022). In this variation, anchor member (138) is titled upward at its distal end, similar to the ski-tip anchor shown in FIGS. 3A-3K. This tilting may, for example, help anchor member (138) to tent or otherwise manipulate tissue during use. Of course, other variations of anchor members may not be tilted, and may have alternate geometries (e.g., a corkscrew geometry, etc.).

FIGS. 11A-11C provide an enlarged view of distal portion (124) of device (120), as the device secures arterial wall tissue. In FIG. 11A, a retainer (1102), similar to retainers (302) and (402) shown in FIGS. 3A and 4A, respectively, has been deployed from a retainer opening (1104) in anchor member (138). A retainer may be useful, for example, in helping to position and/or stabilize anchor member (138), and/or to accurately position device (120) with respect to the tissue. Here, retainer (1102) has been deployed via actuator (132) swinging outwardly about retainer pivot (1103) (depicted as a slot within anchor member (138)), although other appropriate deployment mechanisms may also be used. While the retainer shown in FIG. 11A is in the form of a hypotube connected to actuator (132) via a wire (not shown), other appropriate retainers may be used, as described previously. Also shown in FIG. 11A is a cable tip (1106), which may be used to maintain the retainer in the retainer opening in its undeployed position when desirable, as described previously.

After retainer (1102) has been deployed, device (120) may be pulled proximally, so that anchor member (138) contacts the inner surface of lumen wall (1100), as shown in FIG. 11B. Also shown there is tissue-piercing member port (137) within the subcutaneous tissue. In this position, blood generally will not flow through the tissue-piercing member port to the marker port. As a result, the operator has a visual indication that delivery guide (136) is no longer in the lumen (1024). In this way, proper positioning of the device may be facilitated. As anchor member (138) contacts the inner surface of lumen wall (1100), it deforms at least a portion of the tissue, causing it to tent slightly. This effect allows the tissue to conform to the shape of the anchor, which may offer a way to control the shape of the tissue-piercing member path. In this variation, the anchor member may effectively immobilize a portion of the tissue, in preparation for advancing a tissue-piercing member therethrough.

FIGS. 12A and 12B show the formation of a tract through tissue. First, FIG. 12A depicts the advancement of a tissue-piercing member (1200) into the lumen wall (1100). As shown, tissue-piercing member (1200) enters the lumen wall at a first location (1202) and is advanced laterally into the lumen wall. Notably, tissue-piercing member (1200) has a slight curve. However, other variations of tissue-piercing members may be more curved and/or may have multiple curves, or may be straight. After tissue-piercing member (1200) has been advanced into lumen wall (1100) as shown in FIG. 12A, device (120) may be maneuvered such that the tissue is manipulated to follow the contour of anchor member (138). As described previously, the anchor member may be sized and shaped (e.g., by varying the length and angles throughout) to help ensure a constant contact between the tissue and the anchor member, which allows for more control over the position of the tissue with respect to the tissue-piercing member. Retainer (1102) may act to further secure the positioning of the lumen wall (1100) with respect to the tissue-piercing member. This may cause anchor member (138) to further tent the tissue, and cause the tissue-piercing member (1200) to be redirected from a first direction to a second direction, or from a second direction to a third direction, as the case may be. The diameter of the tract that is formed may, for example, be about equal to the diameter of the tissue-piercing member, and may be, for example, from about 0.5 millimeter to about 2 millimeters (e.g., from about 1 millimeter to about 1.5 millimeters, such as about 1.1 millimeters).

The length of a tract may be any suitable or desirable length. In some variations, the length may be selected to help facilitate relatively rapid sealing of the tract. For example, when the devices and methods described here are used with the vasculature, a longer tract may be desirable, as it is believed that a longer tract may expose helpful biological factors (e.g., growth factors, tissue factors, etc.) that may aid in sealing the tract. This may also be the case with other tissue as well. In addition, a longer tract will have a larger area for mechanical pressure to act on, which may cause the tract to seal more quickly. For example, the tract may seal in 12 minutes or less, 9 minutes or less, 6 minutes or less, 3 minutes or less, etc., reducing the duration of any external compression or pressure that may be needed. In some variations, the length of a tract may be greater than the thickness of a tissue wall in which the tract is formed (e.g., in the location of the tissue wall where the tract is formed, or relative to the average thickness of the tissue wall). A tract may have a height that is equal to the thickness of a tissue wall in which the tract is formed, or in some cases, a tract may have a height that is shorter than the tissue wall thickness. For example, a tract may be formed to deposit one or more therapeutic agents into an interior section of a portion of tissue as previously described. In certain variations in which a tract is being formed in a vessel wall, a portion (e.g., a minority or a majority) of the tract may traverse the vessel wall substantially parallel to a longitudinal axis of the vessel wall.

FIG. 12B shows tissue-piercing member (1200) being further advanced into and through lumen wall (1100), until tissue-piercing member (1200) enters lumen (1024). As tissue-piercing member (1200) is advanced into lumen (1024), a flash of blood may be visualized, either through a marker port, or through an opening in the plunger, as described above. In this way, proper positioning of tissue-piercing member (1200) within lumen (1024) may be confirmed. If further advancement of tissue-piercing member (1200) does not result in entry into the lumen (e.g., if calcification prevents proper tissue-piercing member redirection, or if there is unfavorable anatomy or device positioning, etc.), then device (120) may be withdrawn proximally until side port (1019) is exposed outside the body. At this point a decision may be made to try with another device, or to use a standard arteriotomy procedure (in the case where the tissue is an artery).

In some variations, one or more of the devices and/or methods described here may be used to form one or more tracts in rotated tissue. For example, a method may comprise positioning a device adjacent a portion of a tissue wall, rotating the portion of the tissue wall (e.g., using the device), and advancing a tissue-piercing member through the rotated tissue to form the tract. The rotating may help to position the tissue-piercing member relative to the tissue wall. The tissue may be rotated in either direction about a tissue circumference (e.g., from 0° to 360°, from 0° to 180°, from 0° to 45°, from 45° to 90°, etc.). However, the tissue need not be rotated a significant amount (e.g., the tissue may be rotated 1° , 5°, 10°, 15°, etc.) and the entire tissue thickness need not be rotated.

In some variations, a portion of tissue may only be rotated once, while in other variations, it may be rotated multiple times (e.g., in the same direction or in different directions). Rotation of tissue prior to and/or during tract formation may be useful to effect a desirable tissue-piercing member location, which may in turn be useful for forming a tract having suitable thicknesses of tissue on either side. This may help ensure that the tract is robust enough to withstand repetitive insertion of various tools. In addition, having sufficient tissue thickness on either side of the tract may help the tract seal more quickly. Initial positioning of the tissue-piercing member away from one or more surfaces of the tissue wall may also help with the formation of a longer tract, which may be useful in ensuring more rapid sealing. The portion of tissue may alternatively or additionally be manipulated in one or more other appropriate ways and in some cases, a vacuum may be applied to the portion of tissue. Methods of manipulating tissue and/or applying a vacuum to tissue are described, for example, in U.S. patent application Ser. Nos. 11/873,957 (published as US 2009/0105744 A1) and 12/507,038 (filed on Jul. 21, 2009), both of which were previously incorporated herein by reference in their entirety.

Some variations of the devices described here may comprise one or more heating elements, electrodes, and/or sensors (e.g., Doppler, temperature sensors, pressure sensors, nerve sensors, blood flow sensors, ultrasound sensors, etc.), one or more drug delivery ports along a surface thereof, one or more radiopaque markers to facilitate visualization, or the like. As an example, in some variations, a device may comprise one or more radiopaque materials (e.g., in one or more portions of the device) that may be used to help monitor tract formation. For example, a tissue-piercing member may be made of one or more radiopaque materials or may include radiopaque markings that render the tissue-piercing member visible under X-ray fluoroscopy. In certain variations in which a device comprises one or more sensors, the device may be used to sense at least one useful parameter, such as temperature, pressure, tissue identification or location (e.g., nerves or various anatomical structures), and/or blood flow within a vessel. For example, if the parameter is blood flow within a vessel, the device may be repositioned if blood flow within a vessel is detected.

In some variations, the devices may comprise one or more energy applicators, and may be used to apply energy to tissue. This may, for example, help to seal the tissue. The energy may come from any suitable energy source (e.g., energy selected from the group consisting of ultrasound, radiofrequency (RF), light, magnetic, or combinations thereof). Additionally, certain variations of the devices may comprise one or more cameras (e.g., to facilitate direct visualization). The camera or cameras may or may not have a corresponding light or illumination source, and may be included at any suitable location on a device.

In some variations, a component of a device may, for example, include one or more relatively soft features for contacting a skin surface. As an example, a component of a device may include an inflatable member, such as a relatively soft balloon, that contacts a skin surface when the device is in use. Alternatively or additionally, a component of a device may comprise one or more springs that contact a skin surface when the device is in use (e.g., to provide sufficient tension against the skin surface for isolating a portion of tissue).

Of course, a tissue-piercing member may be advanced through a tissue wall in any appropriate manner, and may be used to form a tract having any shape that is suitable for the procedure being performed. For example, a tract may have a gently sloping shape, may be more angular, may be diagonal, or may have one or more diagonal portions. In some variations, a tract may comprise one or more sloped regions, one or more flat regions, and/or one or more regions that are substantially parallel to a longitudinal axis of a tissue wall in which the tract is formed. In certain variations in which a tract is formed in a vessel wall (e.g., an artery wall), the tract may comprise one or more regions that are substantially parallel to a longitudinal axis of a lumen of the vessel. In some variations, a tissue-piercing member may be configured to advance into tissue along an undulating path, and may thereby form an undulating tract through the tissue. The undulating tract may, for example, have a greater surface area than tracts formed by other tissue-piercing members that follow a relatively straight path. This greater surface area may allow for the tract to self-seal relatively easily. The extent of undulation in a tract may be subtle or substantial. Other configurations of tracts (e.g., sawtooth tracts, oscillating tracts, etc.) may also be formed, as suitable for the particular application at hand.

FIGS. 13A-13E depict examples of tracts that may be formed through an arterial wall (1300). As illustrated in FIG. 13A, arterial wall (1300) may have three layers, the intima (1306), the media (1304), and the adventitia (1302). Tracts that are formed through the arterial wall may traverse through these three layers in a variety of ways. For example, tract (1320) shown in FIG. 13B is substantially straight, where the angles of entry into each lamina (α₂₁), (α₂₂), and (α₂₃) are substantially equal, and may be, for example, from about 6° to about 15°, or from about 3° to about 30°, or from about 35° to about 50°. However, some tracts may have one or more inflections throughout the layers of the arterial wall (1300). FIG. 13C depicts a tract (1321) that has three inflection points, where the three angles of entry (α₂₁), (α₂₂), and (α₂₃) are different from each other. For example, angle (α₂₁) may be about 70°, angle (α₂₂) may be about 45°, and angle (α₂₃) may be about 8°.

These angles (α₂₁), (α₂₂), and (α₂₃) may vary depending on the tissue wall through which the tract is formed, and while some angles may be optimal for forming tracts in one kind of tissue, the same angles may not be suitable for forming tracts in a second kind of tissue. For example, to form a self-sealing tract through arterial wall (1300), it may be desirable to enter the lumen of the artery with a relatively small angle of entry (α₂₃) from the intima, for example, from about 6° to about 15°, or from about 3° to about 30°.

In some cases, it may be desired to form a tract where the majority of the tract resides in one layer of artery wall (1300). FIG. 13D illustrates one variation of a tract (1322) where a substantial portion of the tract is in the media (1304) layer. As shown there, the angles of entry (α₂₁), (α₂₂), and (α₂₃) may also vary as described above. FIG. 13E depicts another variation of a tract (1323) that has inflection points that are not along the layers of the artery wall (1300). For example, inflection angles (α₂₄), (α₂₅), and (α₂₆) may occur anywhere along the tract (1323) as desirable. Inflection angles (α₂₄), (α₂₅), and (α₂₆) may be the same or different, and in some tissues, it may be desirable for inflection angle(a₂₆) to be smaller than inflection angles (a₂₅). la 1 and la 1 Inflection angles (α₂₄), (α₂₅), and (α₂₆) may be, for example, from about 6° to about 15°, or from about 3° to about 30°, or from about 35° to about 50°, from about 60° to about 90°, or from about 75° to about 120°, or from about 120° to about 180°.

As described above, a tract may be self-sealing. In some cases, tract angles such as those described above may be selected to help form a self-sealing tract. A self-sealing tract does not need interventional devices or methods to help it seal—rather, it seals by itself. For example, a self-sealing tract does not need a plug, energy, sealants, clips, sutures, or the like to help it seal. In some variations, a tract may seal when different regions of the tissue defining the tract (e.g., opposing and/or overlapping regions of tissue) come together to seal. In certain variations, the angle between a tissue tract and a lumen at the point of entry of the tissue tract into the lumen may be relatively shallow (e.g., from about 6° to about 20°, from about 6° to about 15°, from about 9° to about 12°). This may, for example, enhance the self-sealing ability of the tract (e.g., because the tract may be relatively long within the tissue wall, and may thereby have substantial surface area for self-sealing). In some variations, pressure may be applied to a self-sealing tract after the tract has been formed (e.g., to make the tract seal even more quickly). In certain variations in which a tract does not self-seal within a certain amount of time (e.g., fifteen minutes or less, ten minutes or less, five minutes or less, two minutes or less, one minute or less), pressure, such as manual pressure, may be applied for a relatively short amount of time (e.g., two minutes or less) to help the tract to seal.

In some variations, one or more tracts may be formed in a tissue having one or more irregular tissue surfaces. The irregular surfaces may be in the form of, for example, undulations, bends, curves, recesses, protrusions, any combination of these, or the like. Methods of forming tracts in irregular tissue surfaces are described, for example, in U.S. patent application Ser. No. 11/873,957 (published as US 2009/0105744 A1), which was previously incorporated herein by reference in its entirety.

In some variations, kits may incorporate one or more (e.g., 2, 3, 4, 5) of the devices and/or device components described here. In certain variations, the kits may include one or more of the devices for forming a tract through tissue described here, one or more of the device components described here (e.g., tissue-piercing members), and/or one or more additional tools. For example, the tools may be those that are advanced through the tract during the performance of a procedure (e.g., guidewires, scissors, grippers, ligation instruments, etc.), one or more supplemental tools for aiding in closure (e.g., an energy delivering device, a closure device, and the like), one or more tools for aiding in the procedure (e.g., gastroscope, endoscope, cameras, light sources, etc.), combinations thereof, and the like. In some variations, a kit may include one or more (e.g., 2, 3, 4, 5) sheath introducers, such as 5 Fr or 6 Fr sheath introducers. Of course, instructions for use may also be provided with the kits.

While devices, methods, and kits have been described in some detail here by way of illustration and example, such illustration and example is for purposes of clarity of understanding only. It will be readily apparent to those of ordinary skill in the art in light of the teachings herein that certain changes and modifications may be made thereto without departing from the spirit and scope of the appended claims.

What is claimed is:

1. A method for forming a tract in a tissue wall having an interior surface and an exterior surface, the method comprising:
advancing an anchor member through the tissue wall and into a lumen defined by the tissue wall, the anchor member comprising a proximal portion, a distal portion, and an intermediate portion therebetween, wherein the proximal and intermediate portions are angled with respect to each other and the intermediate and distal portions are angled with respect to each other;
positioning the anchor member so that the intermediate portion contacts the interior surface of the tissue wall and the distal portion is angled toward the interior surface of the tissue wall; and
advancing a tissue-piercing member into the tissue wall while the intermediate portion is in contact with the interior surface of the tissue wall, to form a tract in the tissue wall.

2. The method of claim 1, wherein the distal portion of the anchor member lifts a portion of the tissue wall when the intermediate portion of the anchor member is in contact with the interior surface of the tissue wall.

3. The method of claim 1, wherein the method comprises using the anchor member to stabilize the tissue wall prior to advancement of the tissue-piercing member into the tissue wall.

4. A device for forming a tract in tissue comprising:
a guide;
a tissue-piercing member slidably housed within the guide and deployable from the guide through an opening in the guide; and
an anchor member coupled to or integral with the guide, the anchor member comprising a first elongated portion, a second elongated portion that is angled with respect to the first elongated portion, and a third elongated portion that is angled with respect to the second elongated portion,
wherein the first elongated portion defines a first plane and the second elongated portion defines a second plane, and wherein the first and second planes have a first angle of about 1° to about 175° therebetween.

5. The device of claim 4, wherein the first angle is about 5° to about 30°.

6. The device of claim 4, wherein the first angle is about 5° to about 20°.

7. The device of claim 4, wherein the first angle is about 5° to about 15°.

8. The device of claim 4, wherein the first angle is about 12°.

9. The device of claim 4, wherein the first angle is about 5° to about 10°.

10. The device of claim 4, wherein the first elongated portion has a length of about 2 millimeters to about 6 millimeters.

11. The device of claim 10, wherein the first elongated portion has a length of about 4 millimeters.

12. The device of claim 4, wherein the tissue-piercing member has a first longitudinal axis and the third elongated portion has a second longitudinal axis that forms a second angle of about 6° to about 30° with the first longitudinal axis upon deployment of the tissue-piercing member from the guide.

13. The device of claim 4, wherein the tissue-piercing member comprises a needle.

14. The device of claim 13, wherein the needle is hollow.

15. The device of claim 4, wherein the third elongated portion defines a third plane, and wherein the second and third planes have a second angle of about 6° to about 25° therebetween.

16. The device of claim 15, wherein the second angle is from about 10° to about 20°.

17. The device of claim 4, wherein the anchor member extends distally from the guide.

18. A device for forming a tract in tissue comprising:
a guide;
a tissue-piercing member slidably housed within the guide and deployable from the guide through an opening in the guide; and
an anchor member coupled to or integral with the guide, the anchor member comprising first, second, and third elongated portions, a first curved portion between the first and second elongated portions, and a second curved portion between the second and third elongated portions,
wherein the first curved portion defines a first plane and the second curved portion defines a second plane that is angled with respect to the first plane.

19. The device of claim 18, wherein the first and second planes have an angle of about 1° to about 175° therebetween.

20. The device of claim 18, wherein the first curved portion has a radius of curvature of about 0.1 millimeter to about 2 millimeters.

21. The device of claim 20, wherein the second curved portion has a radius of curvature of about 0.1 millimeter to about 2 millimeters.

22. The device of claim 18, wherein the anchor member is flexible.

23. The device of claim 18, wherein the anchor member further comprises a guide eye sheath.

24. The device of claim 18, wherein the anchor member further comprises an attachable guidewire.

25. The device of claim 18, wherein the tissue-piercing member comprises a needle.

26. The device of claim 25, wherein the needle is hollow.

27. The device of claim 18, wherein the opening in the guide is located proximal to a distal end of the anchor member.

28. A method for forming a tract in a tissue wall having an interior surface and an exterior surface, the method comprising:
advancing an anchor member through the tissue wall, the anchor member comprising first, second, and third elongated portions, a first curved portion between the first and second elongated portions, and a second curved portion between the second and third elongated portions, the first curved portion defining a first plane and the second curved portion defining a second plane that is angled with respect to the first plane;
contacting the anchor member with the interior surface of the tissue wall; and
advancing a tissue-piercing member into the tissue wall while the anchor member is in contact with the interior surface of the tissue wall, to form a tract in the tissue wall.

29. The method of claim 28, wherein the tissue comprises a vessel and the method comprises advancing the anchor member into a lumen of the vessel.

30. The method of claim 29, wherein the vessel comprises an artery.

31. The method of claim 28, wherein the tissue-piercing member has a first longitudinal axis and the third elongated portion of the anchor member has a second longitudinal axis, and the first and second longitudinal axes form an angle therebetween.

32. The method of claim 31, wherein the angle between the first and second longitudinal axes is from about 6° to about 30° when the tissue-piercing member is advanced through the tissue wall.

33. The method of claim 31, further comprising advancing the tissue-piercing member into a lumen defined by the tissue wall, wherein the angle between the first and second longitudinal axes is from about 6° to about 30° upon entry of the tissue-piercing member into the lumen.

34. A device for forming a tract through tissue comprising:
a guide;
an anchor member coupled to or integral with a distal portion of the guide;
a marker port coupled to or integral with a proximal portion of the guide and having a first lumen;
a tissue-piercing member deployable from the guide; and
a pushing member configured to deploy the tissue-piercing member from the guide,
wherein the tissue-piercing member comprises a first tubular member comprising a wall portion having a plurality of apertures therethrough, such that the tissue-piercing member is in fluid communication with the marker port.

35. The device of claim 34, wherein the tissue-piercing member remains in fluid communication with the marker port when translated by the pushing member.

36. A device for forming a tract through tissue comprising:
a marker port comprising a lumen; and
a tissue-piercing member comprising a tubular member comprising a wall portion having a plurality of apertures therethrough,
wherein at least a portion of the tissue-piercing member passes through the lumen of the marker port.

37. A method of forming a tract through tissue using a device comprising an anchor member, a marker port, and a tissue-piercing member at least partially disposed within the marker port and comprising a tubular member comprising a wall portion having a plurality of apertures therethrough, the method comprising:
advancing the anchor member into a vessel wall defining a first lumen until blood flows through the marker port to indicate that the anchor member has entered the first lumen; and
advancing the tissue-piercing member into the vessel wall while the anchor member is disposed within the first lumen.

38. The method of claim 37, wherein the tissue-piercing member comprises a second lumen and wherein the method further comprises advancing a guidewire through the second lumen.

39. The method of claim 37, wherein the tissue-piercing member is advanced into the vessel wall by pushing on a pushing member that is in contact with the tissue-piercing member.

40. A device for forming a tract through tissue comprising:
a guide;
a tissue-piercing member deployable from the guide;
an anchor member coupled to or integral with the guide; and
a sheath coupled to the anchor member,
wherein the sheath comprises a flexible elongated member comprising a distal portion comprising a first region having a first cross-sectional diameter and a second region that is integral with the first region, the second region having a second cross-sectional diameter that is different from the first cross-sectional diameter.

41. A method of making a device for forming a tract through tissue comprising:
forming a sheath using a bump extrusion process; and
coupling the sheath to an anchor member that is coupled to or integral with a guide configured for deployment of a tissue-piercing member therefrom.

42. The method of claim 41, wherein the guide comprises a lumen and a tissue-piercing member slidably disposed within the lumen.

43. A system for forming a tract through tissue comprising:
a device comprising a guide, an anchor member coupled to or integral with the guide, a pushing member, and a tissue-piercing member deployable from the guide by pushing on the pushing member; and
a syringe,
wherein the pushing member comprises an elongated member having a handle portion at its proximal end, and wherein the syringe is configured to couple with the handle portion.

44. The system of claim 43, wherein the handle portion of the pushing member comprises a female connector and the syringe comprises a male connector configured to couple to the female connector.

45. A device for forming a tract in tissue comprising:
a guide;
a tissue-piercing member slidably housed within the guide and deployable through an opening in the guide;
an anchor member coupled to or integral with the guide;
a retainer configured to be actuated from a position in which the retainer is aligned with the anchor member to a position in which the retainer extends from the anchor member; and
a tensioning apparatus comprising a tensioning member configured to actuate the retainer, and a tubular member housing a portion of the tensioning member, wherein the tubular member is coupled to or integral with the guide.

46. The device of claim 45, wherein the tensioning member is coupled to the retainer.

47. A device for forming a tract in tissue comprising:
a guide;
a tissue-piercing member slidably housed within the guide and deployable through an opening in the guide;
an anchor member coupled to or integral with the guide;
a retainer configured to be actuated from a position in which the retainer is aligned with the anchor member to a position in which the retainer extends from the anchor member; and
a tensioning apparatus comprising a tensioning member configured to actuate the retainer and a semitubular member housing a portion of the tensioning member,
wherein the semitubular member is coupled to or integral with the guide.

48. The device of claim 47, wherein the tensioning member is coupled to the retainer.

49. A device for forming a tract in tissue comprising:
a guide;
a tissue-piercing member slidably housed within the guide and deployable through an opening in the guide;
an anchor member coupled to or integral with the guide;
a retainer configured to be actuated from a position in which the retainer is aligned with the anchor member to a position in which the retainer extends from the anchor member; and
a tensioning member coupled to the retainer and configured to actuate the retainer,
wherein a first portion of the tensioning member is disposed along an outer surface of the guide, a second portion of the tensioning member passes through an opening in a wall portion of the guide, and a third portion of the tensioning member is disposed within a lumen of the guide.

50. The device of claim 49, wherein the portion of the guide housing the tensioning member has a non-circular cross-section.

51. The device of claim 50, wherein the portion of the guide housing the tensioning member has an elliptical cross-section.

52. The device of claim 49, wherein the portion of the guide housing the tensioning member is sized and shaped to house both the tensioning member and the tissue-piercing member.
